(19) United States
(12) Patent Application Publication
(10) Pub. No.: US 2021/0292761 A1
(43) Pub. Date: Sep. 23, 2021

(54) COMPOSITIONS COMPRISING CIRCULAR POLYRIBONUCLEOTIDES AND USES THEREOF

(71) Applicant: FLAGSHIP PIONEERING INNOVATIONS VI, LLC, Cambridge, MA (US)

(72) Inventors: Avak KAHVEJIAN, Lexington, MA (US); Nicholas McCartney PLUGIS, Boston, MA (US); Alexandra Sophie DE BOER, Somerville, MA (US); Morag Helen STEWART, Boston, MA (US); Catherine CIFUENTES-ROJAS, Brookline, MA (US); Ki Young PAEK, Worcester, MA (US)

(21) Appl. No.: 17/262,591

(22) PCT Filed: Jul. 24, 2019
(86) PCT No.: PCT/US2019/043272

§ 371 Date: Jan. 22, 2021

Related U.S. Application Data
(60) Provisional application No. 62/702,714, filed on Jul. 24, 2018.
(60) Provisional application No. 62/823,569, filed on Mar. 25, 2019.
(60) Provisional application No. 62/863,670, filed on Jun. 19, 2019.

(51) Int. Cl. C12N 15/115; A61K 31/7088; A61K 31/704; C12N 15/113

(52) U.S. Cl. CPC C12N 15/115; A61K 31/7088; A61K 31/704; C12N 15/113; C12N 2310/14; C12N 15/1138; C12N 2310/16; C12N 2310/532; C12N 15/1131

(57) ABSTRACT

This invention relates generally to pharmaceutical compositions and preparations of circular polyribonucleotides and uses thereof.

### CROSS-REFERENCE TO RELATED APPLICATIONS

The present application claims priority to and benefit from U.S. Provisional Application Nos. 62/702,714, filed Jul. 24, 2018; 62/823,569, filed Mar. 25, 2019; and 62/863,670, filed Jun. 19, 2019, the entire contents of each of which are herein incorporated by reference.

### BACKGROUND

Certain circular polyribonucleotides are ubiquitously present in human tissues and cells, including tissues and cells of healthy individuals.

### SUMMARY

The present invention described herein includes compositions comprising circular polyribonucleotides and methods of their use.

In some aspects, a method of binding a target in a cell comprises providing a translation incompetent circular polyribonucleotide comprising an aptamer sequence, wherein the aptamer sequence has a secondary structure that binds the target; and delivering the translation incompetent circular polyribonucleotide to the cell, wherein the translation incompetent circular polyribonucleotide forms a complex with the target detectable at least 5 days after delivery. In some embodiments, the target is selected from the group consisting of a nucleic acid molecule, a small molecule, a protein, a carbohydrate, and a lipid. In some embodiments, the target is a gene regulation protein. In some embodiments, the gene regulation protein is a transcription factor. In some embodiments, the nucleic acid molecule is a DNA molecule or an RNA molecule. In some embodiments, the complex modulates gene expression. In some embodiments, the complex modulates directed transcription of the DNA molecule, epigenetic remodeling of the DNA molecule, or degradation of the DNA molecule. In some embodiments, the complex modulates degradation of the target, translocation of the target, or target signal transduction. In some embodiments, the gene expression is associated with pathogenesis of a disease or condition. In some embodiments, the complex is detectable at least 7, 8, 9, or 10 days after delivery. In some embodiments, the translation incompetent circular polyribonucleotide is present at least five days after delivery. In some embodiments, the translation incompetent circular polyribonucleotide is present at least 6, 7, 8, 9, or 10 days after delivery. In some embodiments, the translation incompetent circular polyribonucleotide is an unmodified translation incompetent circular polyribonucleotide. In some embodiments, the translation incompetent circular polyribonucleotide has a quasi-double-stranded secondary structure. In some embodiments, the aptamer sequence further has a tertiary structure that binds the target. In some embodiments, the cell is a eukaryotic cell. In some embodiments, the eukaryotic cell is a human cell.

In some aspects, a method of binding a transcription factor in a cell comprises providing a translation incompetent circular polyribonucleotide comprising an aptamer sequence that binds the transcription factor; and delivering the translation incompetent circular polyribonucleotide to the cell, wherein the translation incompetent circular polyribonucleotide forms a complex with the transcription factor and modulates gene expression.

In some aspects, a method of sequestering a transcription factor in a cell comprises providing a translation incompetent circular polyribonucleotide comprising an aptamer sequence that binds the transcription factor; and delivering the translation incompetent circular polyribonucleotide to the cell, wherein the translation incompetent circular polyribonucleotide sequesters the transcription factor by binding the transcription factor to form a complex in the cell. In some embodiments, cell viability decreases after formation of the complex.

In some aspects, a method of sensitizing a cell to a cytotoxic agent comprises providing a translation incompetent circular polyribonucleotide comprising an aptamer sequence that binds a transcription factor; and delivering the cytotoxic agent and the translation incompetent circular polyribonucleotide to the cell, wherein the translation incompetent circular polyribonucleotide forms a complex with the transcription factor in the cell; thereby sensitizing the cell to the cytotoxic agent compared to a cell lacking the translation incompetent circular polyribonucleotide. In some embodiments, the sensitizing the cell to the cytotoxic agent results in decreased cell viability after the delivering of the cytotoxic agent and the translation incompetent circular polyribonucleotide. In some embodiments, the decreased cell viability is decreased by 40% or more at least two days after the delivering of the cytotoxic agent and the translation incompetent circular polyribonucleotide.

In some aspects, a method of binding a pathogenic protein in a cell comprises: providing a translation incompetent circular polyribonucleotide comprising an aptamer sequence that binds the pathogenic protein; and delivering the translation incompetent circular polyribonucleotide to the cell, wherein the translation incompetent circular polyribonucleotide forms a complex with the pathogenic protein for degrading the pathogenic protein.

In some aspects, a method of binding a ribonucleic acid molecule in a cell comprises: providing a translation incompetent circular polyribonucleotide comprising a sequence complementary to a sequence of the ribonucleic acid molecule; and delivering the translation incompetent circular polyribonucleotide to the cell, wherein the translation incompetent circular polyribonucleotide forms a complex with the ribonucleic acid molecule.

In some aspects, a method of binding genomic deoxyribonucleic acid molecule in a cell comprises providing a translation incompetent circular polyribonucleotide comprising an aptamer sequence that binds the genomic deoxyribonucleic acid molecule; and delivering the translation incompetent circular polyribonucleotide to the cell, wherein the translation incompetent circular polyribonucleotide forms a complex with the genomic deoxyribonucleic acid molecule and modulates gene expression.

In some aspects, a method of binding a small molecule in a cell comprises providing a translation incompetent circular polyribonucleotide comprising an aptamer sequence that binds the small molecule; and delivering the translation incompetent circular polyribonucleotide to the cell, wherein the translation incompetent circular polyribonucleotide forms a complex with the small molecule and modulates a cellular process. In some embodiments, the small molecule is an organic compound having a molecular weight of no more than 900 daltons and modulates a cellular process. In some embodiments, the small molecule is a drug. In some embodiments, the small molecule is a fluorophore. In some embodiments, the small molecule is a metabolite.

In some aspects, a composition comprises a translation incompetent circular polyribonucleotide comprising an aptamer sequence, wherein the aptamer sequence has a secondary structure that binds a target.

In some aspects, a pharmaceutical composition comprises a translation incompetent circular polyribonucleotide comprising an aptamer sequence, wherein the aptamer sequence has a secondary structure that binds the target; and a pharmaceutically acceptable carrier or excipient.

In some aspects, a cell comprises the translation incompetent circular polyribonucleotide as described herein.

In some aspects, a method of treating a subject in need thereof comprises administering the composition as described herein or the pharmaceutical composition as described herein.

In some aspects, a polynucleotide is a polynucleotide that encodes the translation incompetent circular polyribonucleotide of as described herein.

In some aspects, a method is a method of producing the translation incompetent circular polyribonucleotide as described herein.

In some aspects, a pharmaceutical composition comprises a circular polyribonucleotide comprising a binding site that binds a target, e.g., a RNA, DNA, protein, membrane of cell etc.; and a pharmaceutically acceptable carrier or excipient; wherein the target and the circular polyribonucleotide form a complex, and wherein the target is a not a microRNA. In some aspects, a pharmaceutical composition comprises a circular polyribonucleotide comprising: a first binding site that binds a first target, and a second binding site that binds a second target; and a pharmaceutically acceptable carrier or excipient; wherein the first binding site is different than the second binding site, and wherein the first target and the second target are both a microRNA. In some embodiments, the binding site comprises an aptamer sequence. In some embodiments, the first binding site comprises a first aptamer sequence and the second binding site comprises a second aptamer sequence. In some embodiments, the aptamer sequence has a secondary structure that binds the target. In some embodiments, the first aptamer sequence has a secondary structure that binds the first target and the second aptamer sequence has a secondary structure that binds the second target. In some embodiments, the binding site is a first binding site and the target is a first target. In some embodiments, the circular polyribonucleotide further comprises a second binding site that binds to a second target. In some embodiments, the first target comprises a first circular polyribonucleotide (circRNA)-binding motif. In some embodiments, the second target comprises a second circular polyribonucleotide (circRNA)-binding motif. In some embodiments, the first target, the second target, and the circular polyribonucleotide form a complex. In some embodiments, the first and second targets interact with each other. In some embodiments, the complex modulates a cellular process. In some embodiments, the first and second targets are the same, and the first and second binding sites bind different binding sites on the first target and the second target. In some embodiments, the first target and the second target are different. In some embodiments, the circular polyribonucleotide further comprises one or more additional binding sites that bind a third or more targets. In some embodiments, one or more targets are the same and one or more additional binding sites bind different binding sites on the one or more targets. In some embodiments, formation of the complex modulates a cellular process. In some embodiments, the circular polyribonucleotide modulates a cellular process associated with the first or second target when contacted to the first and second targets. In some embodiments, the first and second targets interact with each other in the complex. In some embodiments, the cellular process is associated with pathogenesis of a disease or condition. In some embodiments, the cellular process is different than translation of the circular polyribonucleic acid. In some embodiments, the first target comprises a deoxyribonucleic acid (DNA) molecule, and the second target comprises a protein. In some embodiments, the complex modulates directed transcription of the DNA molecule, epigenetic remodeling of the DNA molecule, or degradation of the DNA molecule. In some embodiments, wherein the first target comprises a first protein, and the second target comprises a second protein. In some embodiments, wherein the complex modulates degradation of the first protein, translocation of the first protein, or signal transduction, or modulates a native protein function, inhibits or modulates formation of a complex formed by direct interaction between the first and second proteins. In some embodiments, the first target or the second target is a ubiquitin ligase. In some embodiments, the first target comprises a first ribonucleic acid (RNA) molecule, and the second target comprises a second RNA molecule. In some embodiments, the complex modulates degradation of the first RNA molecule. In some embodiments, the first target comprises a protein, and the second target comprises a RNA molecule. In some embodiments, the complex modulates translocation of the protein or inhibits formation of a complex formed by direct interaction between the protein and the RNA molecule. In some embodiments, the first target is a receptor, and the second target is a substrate of the receptor. In some embodiments, the complex inhibits activation of the receptor.

In some aspects, a pharmaceutical composition comprises a circular polyribonucleotide comprising a binding site that binds a target; and a pharmaceutically acceptable carrier or excipient; wherein the circular polyribonucleotide is translation incompetent or translation defective, and wherein the target is not a microRNA. In some aspects, a pharmaceutical composition comprises a circular polyribonucleic acid comprising a binding site that binds a target, wherein the target comprises a ribonucleic acid (RNA)-binding motif; and a pharmaceutically acceptable carrier or excipient; wherein the circular polyribonucleotide is translation incompetent or translation defective, and wherein the target is a microRNA. In some embodiments, the binding site comprises an aptamer sequence having a secondary structure that binds the target. In some embodiments, the target comprises a DNA molecule. In some embodiments, binding of the target to the circular polyribonucleotide modulates interference of transcription of a DNA molecule. In some embodiments, the target comprises a protein. In some embodiments, binding of the target to the circular polyribonucleotide modulates interaction of the protein with other molecules. In some embodiments, the protein is a receptor, and binding of the target to the circular polyribonucleotide activates the receptor. In some embodiments, the protein is a first enzyme, wherein the circular polyribonucleotide further comprises a second binding site that binds to a second enzyme, and wherein binding of the first and second enzymes to the circular polyribonucleotide modulates enzymatic activity of the first and second enzymes. In some embodiments, the protein is a ubiquitin ligase. In some embodiments, the target comprises a messenger RNA (mRNA) molecule. In some embodiments, binding of the target to the circular polyribonucleotide modulates interference of translation of the mRNA molecule. In some embodiments, the target comprises a ribosome. In some embodiments, binding of the target to the circular polyribonucleotide modulates interference of a translation process. In some embodiments, the target comprises a circular RNA molecule. In some embodiments, binding of the target to the circular polyribonucleotide sequesters the circular RNA molecule. In some embodiments, binding of the target to the circular polyribonucleotide sequesters the microRNA molecule.

In some aspects, a pharmaceutical composition comprises a circular polyribonucleotide comprising a binding site that binds to a membrane of a cell (e.g., cell wall membrane, organelle membrane, etc.), wherein the membrane of the cell comprises a ribonucleic acid (RNA)-binding motif; and a pharmaceutically acceptable carrier or excipient. In some embodiments, the binding site comprises an aptamer sequence having a secondary structure that binds the membrane of the cell (e.g., cell wall membrane, organelle membrane, etc.). In some embodiments, the circular polyribonucleotide further comprises a second binding site that binds to a second target, wherein the second target comprises a second RNA-binding motif. In some embodiments, the circular polyribonucleotide binds to the membrane of the cell and the second target. In some embodiments, the circular polyribonucleotide further comprises a second binding site that binds to a second cell target, and wherein binding of the cell target and the second cell target to the circular polyribonucleotide induces a conformational change in the cell target, thereby inducing signal transduction downstream of the cell target. In some embodiments, the circular polyribonucleotide is translation incompetent or translation defective. In some embodiments, circular polyribonucleotide further comprises at least one structural element selected from the group consisting of: a) an encryptogen; b) a splicing element; c) a regulatory sequence; d) a replication sequence; e) a quasi-double-stranded secondary structure; f) a quasi-helical structure; and g) an expression sequence. In some embodiments, the quasi-helical structure comprises at least one double-stranded RNA segment with at least one non-double-stranded segment. In some embodiments, the quasi-helical structure comprises a first sequence and a second sequence linked with a repetitive sequence. In some embodiments, the encryptogen comprises a splicing element. In some embodiments, the circular polyribonucleic acid comprises at least one modified nucleic acid. In some embodiments, the at least one modified nucleic acid is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA), a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some embodiments, the encryptogen comprises at least one modified nucleic acid. In some embodiments, the encryptogen comprises a protein binding site. In some embodiments, the encryptogen comprises an immunoprotein binding site. In some embodiments, the circular polyribonucleic acid has at least 2× lower immunogenicity than a counterpart lacking the encryptogen, as assessed by expression, signaling, or activation of at least one of RIG-I, TLR-3, TLR-7, TLR-8, MDA-5, LGP-2, OAS, OASL, PKR, and IFN-beta In some embodiments, the circular polyribonucleic acid has a size of about 20 bases to about 20 kb. In some embodiments, the circular polyribonucleic acid is synthesized through circularization of a linear polynucleotide. In some embodiments, the circular polyribonucleic acid is substantially resistant to degradation.

In some aspects, a pharmaceutical composition, comprises a circular polyribonucleotide comprising a binding site that binds to a target, wherein the target comprises a ribonucleic acid (RNA)-binding motif; and a pharmaceutically acceptable carrier or excipient, wherein the circular polyribonucleotide comprises at least one modified nucleotide and a first portion that comprises at least about 5, 10, 20, 50, 100, 200, 300, 400, 500, 600, 700, 800, 900, or 1000 contiguous unmodified nucleotides. In some aspects, a pharmaceutical composition, comprises: a circular polyribonucleotide comprising a binding site that binds to a target, wherein the target comprises a ribonucleic acid (RNA)-binding motif; and a pharmaceutically acceptable carrier or excipient, wherein the circular polyribonucleotide comprises at least one modified nucleotide and a first portion that comprises at least about 5, 10, 20, 50, 100, 200, 300, 400, 500, 600, 700, 800, 900, or 1000 contiguous nucleotides, and wherein the first portion lacks pseudouridine or 5′-methylcytidine. In some embodiments, the binding site comprises an aptamer sequence having a secondary structure that binds the target. In some embodiments, the circular polyribonucleotide has a lower immunogenicity than a corresponding unmodified circular polyribonucleotide. In some embodiments, the circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide, as assessed by expression or signaling or activation of at least one of the group consisting of RIG-I, TLR-3, TLR-7, TLR-8, MDA-5, LGP-2, OAS, OASL, PKR, and IFN-beta. In some embodiments, the circular polyribonucleotide has a higher half-life than a corresponding unmodified circular polyribonucleotide. In some embodiments, the circular polyribonucleotide has a half-life that is at least about 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide. In some embodiments, the half-life is measured by introducing the circular polyribonucleotide or the corresponding unmodified circular polyribonucleotide into a cell and measuring a level of the introduced circular polyribonucleotide or corresponding circular polyribonucleotide inside the cell. In some embodiments, the at least one modified nucleotide is selected from the group consisting of: N(6)methyladenosine (m6A), 5′-methylcytidine, and pseudouridine. In some embodiments, the at least one modified nucleic acid is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA), a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some embodiments, at least about 10%, 20%, 30%, 40%, 50%, 60%, 70%, 80%, 90%, 95%, or 99% of nucleotides of the circular polyribonucleotide are modified nucleotides. In some embodiments, the circular polyribonucleotide comprises a binding site that binds to a protein, DNA, RNA, or a cell target, consisting of unmodified nucleotides. In some embodiments, the circular polyribonucleotide comprises an internal ribosome entry site (IRES) consisting of unmodified nucleotides. In some embodiments, the binding site consists of unmodified nucleotides. In some embodiments, the binding site comprises an IRES consisting of unmodified nucleotides. In some embodiments, the first portion comprises a binding site that binds a protein, DNA, RNA, or a cell target. In some embodiments, the the first portion comprises an IRES. In some embodiments, the circular polyribonucleotide comprises one or more expression sequences. In some embodiments, the circular polyribonucleotide comprises the one or more expression sequences and the IRES, and wherein the circular polyribonucleotide comprises a 5′-methylcytidine, a pseudouridine, or a combination thereof outside the IRES. In some embodiments, one or more expression sequences of the circular polyribonucleotide are configured to have a higher translation efficiency than a corresponding unmodified circular polyribonucleotide. In some embodiments, one or more expression sequences of the circular polyribonucleotide have a translation efficiency of that is at least about 0.7, 0.8, 0.9, 1.0, 1.1, 1.2, 1.3, 1.5, 1.6, 1.8, 2, 2.2, 2.5, 2.8, or 3 fold higher than a corresponding unmodified circular polyribonucleotide. In some embodiments, one or more expression sequences of the circular polyribonucleotide have a higher translation efficiency than a corresponding circular polyribonucleotide having a first portion comprising a modified nucleotide. In some embodiments, one or more expression sequences of the circular polyribonucleotide have a higher translation efficiency than a corresponding circular polyribonucleotide having a first portion comprising more than 10% modified nucleotides. In some embodiments, one or more expression sequences of the circular polyribonucleotide have a translation efficiency that is at least about 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding circular polyribonucleotide having a first portion comprising a modified nucleotide. In some embodiments, the translation efficiency is measured either in a cell comprising the circular polyribonucleotide or the corresponding circular polyribonucleotide, or in an in vitro translation system (e.g., rabbit reticulocyte lysate). In some embodiments, the circular polyribonucleotide is the circular polyribonucleotide of any one of the disclosed embodiments.

In some aspects, a method of treatment comprises administering the pharmaceutical composition of any one of of the previously disclosed embodiments to a subject with a disease or condition.

In some aspects, a method of producing a pharmaceutical composition comprises generating the circular polyribonucleotide of any one of the disclosed embodiments.

In some aspects, the composition of any one of the embodiments is formulated in a carrier, e.g., membrane or lipid bilayer.

In some aspects, a method of making the circular polyribonucleotide of any one of disclosed embodiments comprises circularizing a linear polyribonucleotide having a nucleic acid sequence as the circular polyribonucleotide.

In some aspects, an engineered cell comprises the composition of any one of the disclosed embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 illustrates an example circular polyribonucleotide molecular scaffold.

FIG. 2 illustrates an example trans-ribozyme circular polyribonucleotide.

FIG. 3 illustrates a schematic of protein expression by a circular polyribonucleotide.

FIG. 4 illustrates an example circular polyribonucleotide molecular scaffold for lipids, such as membranes.

FIG. 5A illustrates an example circular polyribonucleotide molecular scaffold for DNA.

FIG. 5B illustrates an example circular polyribonucleotide molecular scaffold with a sequence specific DNA binding motif. The circRNA can bind to the major groove of the DNA duplex to form parallel or antiparallel triplex structures based on the orientation of the third strand. Exemplary parallel triplex structures include TA.U, CG.G and CG.C (DNA DNA.RNA). Exemplary antiparallel triplex structures include TA.A, TA.C and CG.G (DNA DNA.RNA).

FIG. 5C illustrates an example circular polyribonucleotide molecular scaffold with a DNA binding motif specific to an enhancer region of the DHFR gene for interference with transcription factor binding and/or mRNA transcription.

FIG. 5D illustrates an example circular polyribonucleotide molecular scaffold with a DNA binding motif specific to an enhancer region of the MEG3 gene for interference with transcription factor binding and/or mRNA transcription.

FIG. 5E illustrates an example circular polyribonucleotide molecular scaffold with a DNA binding motif specific to an enhancer region of the EPS gene for interference with transcription factor binding and/or mRNA transcription.

FIG. 6 illustrates an example circular polyribonucleotide molecular scaffold for RNA.

FIG. 7A illustrates an example circular polyribonucleotide molecular scaffold for target RNAs to sequester and/or degrade target RNAs.

FIG. 7B illustrates an example circular polyribonucleotide molecular scaffold for RNAs and enzymes targeting the RNAs (e.g., decapping enzymes that induce degradation of the RNAs).

FIG. 7C illustrates an example circular polyribonucleotide molecular scaffold for RNA, DNA and protein (e.g., to drive target gene translation).

FIG. 8 illustrates an example circular polyribonucleotide molecular scaffold for protein (e.g., FUS/TDP43/ATXN2, PRPF8, GEMIN5, CUG BP1 and LIN28A).

FIGS. 9A, 9B, and 9C show that the modified circular RNAs bind protein translation machinery in cells.

FIGS. 10A, 10B, and 10C show that modified circular RNAs have reduced binding to immune proteins as assessed by activation of immune related genes (MDA5, OAS, and IFN-beta expression) as compared to unmodified circular RNAs in cells.

FIG. 11 shows that hybrid modified circular RNAs have reduced immunogenicity as compared to unmodified circular RNAs as assessed by RIG-I, MDA5, IFN-beta, and OAS expression in cells.

FIG. 12 demonstrates that a circular RNA aptamer exhibits increased intracellular delivery and enhanced binding to a small molecule target compared to a linear aptamer.

FIG. 13 illustrates binding of a circular RNA containing a protein-binding motif to a target protein.

FIG. 14 demonstrates a small molecule-circular RNA conjugate binds to a protein targeted by the small molecule.

FIG. 15 demonstrates interaction of a circular RNA-small molecule conjugate with a specific bioactive protein.

FIG. 16 illustrates a circRNA with two binding sites that can act as a scaffold, for example, to form a complex with an enzyme (Enz) and a target substrate (substrate), facilitating modification (M) of the target substrate by the enzyme.

FIG. 17 shows images from electrophoretic mobility shift assay (EMSA) demonstrating that RNA with scrambled binding aptamer sequences did not show binding affinity to the p50 subunit of NF-kB, while both linear and circular RNAs with the NF-kB binding aptamer sequence bound to the p50 subunit with similar affinities.

FIG. 18 shows that treatment with circular RNA with the NF-kB binding aptamer sequence led to a decrease in cell viability of A549 cells as compared to its linear counterpart.

FIG. 19 shows co-treatment with linear RNA and doxorubicin (dox) decreased cell viability at day 2 and co-treatment with the circular aptamer and dox resulted in more cell death at both days 1 and 2 in the dox-resistant A549 lung cancer cell line.

FIG. 20 is a schematic showing an exemplary circular RNA that is delivered into cells and tags a target BRD4 protein in the cells for degradation by ubiquitin system.

FIG. 21 shows Western blot images and quantitative chart demonstrating that circular RNA containing thalidomide and JQ1 small molecules was able to degrade BRD4 in cells.

FIG. 22 shows aptamer fluorescence when bound to TO-1 biotin at different time points after delivery of the circular RNA (endless aptamer) or the linear RNA (linear aptamer) to HeLa cell cultures. The fluorescent images (top) show aptamer fluorescence when bound to TO-1 biotin at 6 hours, Day 1, and Day 10 after delivery of the the circular RNA (endless aptamer) or the linear RNA (linear aptamer). The graphs (bottom) show the percentage of fluorescent cells in the HeLa cell cultures at 6 hours, Day 1, Day 3, Day 5, Day 7, Day 10, and Day 12 after delivery of the the circular RNA (endless aptamer), the linear RNA (linear aptamer), or the TO-1 biotin only (control).

FIG. 23 shows HuR bound circular RNAs with a HuR RNA binding aptamer motif and the streptavidin pull-down yielded RNAs with the RNA binding aptamer motifs compared to a circular RNA with no binding aptamer motifs, a circular RNA with a HuR RNA binding aptamer motif, and a circular RNA with an RNA binding aptamer motif.

FIG. 24 shows HuR bound circular RNAs with the HuR DNA binding aptamer motif and the streptavidin pull-down yielded RNAs with the DNA binding aptamer motifs compared to a circular RNA with no binding apatmer motifs, a circular RNA with a HuR DNA binding aptamer motif, and a circular RNA with DNA.

FIG. 25 shows lower secreted protein expression from circular RNA without a HuR binding motif compared to a circular RNA with 1×HuR binding motif, 2×HuR binding motifs, and 3×HuR binding motifs.

### DETAILED DESCRIPTION

This invention relates generally to pharmaceutical compositions and preparations of circular polyribonucleotides and uses thereof.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

### Definitions

As used herein, the term “circRNA” or “circular RNA” or “circular polyribonucleotide” refers to a polyribonucleotide that forms a circular structure through covalent or non-covalent bonds.

As used herein, the term “encryptogen” refers to a nucleic acid sequence of the circular polyribonucleotide that aids in reducing, evading, and/or avoiding detection by an immune cell and/or reduces induction of an immune response against the circular polyribonucleotide.

As used herein, the term “expression sequence” refers to a nucleic acid sequence that encodes a product, e.g., a peptide or polypeptide, or a regulatory nucleic acid.

As used herein, the term “immunoprotein binding site” refers to a nucleotide sequence that binds to an immunoprotein and aids in masking the circular polyribonucleotide as non-endogenous.

As used herein, the term “modified ribonucleotide” refers to a nucleotide with at least one modification to the sugar, the nucleobase, or the internucleoside linkage.

As used herein, the phrase “quasi-helical structure” refers to a higher order structure of the circular polyribonucleotide, wherein at least a portion of the circular polyribonucleotide folds into a helical structure.

As used herein, the phrase “quasi-double-stranded secondary structure” refers to a higher order structure of the circular polyribonucleotide, wherein at least a portion of the circular polyribonucleotide creates a double strand.

As used herein, the term “regulatory sequence” refers to a nucleic acid sequence that modifies an expression product.

As used herein, the term “repetitive nucleotide sequence” refers to a repetitive nucleic acid sequence within a stretch of DNA or throughout a genome. In some embodiments, the repetitive nucleotide sequence includes poly CA or poly TG sequences. In some embodiments, the repetitive nucleotide sequence includes repeated sequences in the Alu family of introns.

As used herein, the term “replication element” refers to a sequence and/or motifs useful for replication or that initiate transcription of the circular polyribonucleotide.

As used herein, the term “selective translation sequence” refers to a nucleic acid sequence that selectively initiates or activates translation of an expression sequence in the circular polyribonucleotide.

As used herein, the term “selective degradation sequence” refers to a nucleic acid sequence that initiates translation of an expression sequence in the circular polyribonucleotide.

As used herein, the term “stagger sequence” refers to a nucleotide sequence that induces ribosomal pausing during translation. In some embodiments, the stagger sequence is a non-conserved sequence of amino-acids with a strong alpha-helical propensity followed by the consensus sequence −D(V/I)ExNPG P, where x is any amino acid.

As used herein, the term “substantially resistant” refers to one that has at least 50%, 60%, 70%, 75%, 80%, 85%, 90%, 95%, 96%, 97%, 98% or 99% resistance as compared to a reference.

As used herein, the term “complex” refers to an association between at least two moieties (e.g., chemical or biochemical) that have an affinity for one another. For example, at least two moieties are a target (e.g., a protein) and a circular RNA molecule.

“Polypeptide” and “protein” are used interchangeably and refer to a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics).

As used herein, the term “binding site” refers to a region of the circular polyribonucleotide that interacts with another entity, e.g., a chemical compound, a protein, a nucleic acid, etc. A binding site can comprise an aptamer sequence.

As used herein, the term “binding moiety” refers to a region of a target that can be bound by a binding site, for example, a region, domain, fragment, epitope, or portion of a nucleic acid (e.g., RNA, DNA, RNA-DNA hybrid), chemical compound, small molecule (e.g., drug), aptamer, polypeptide, protein, lipid, carbohydrate, antibody, virus, virus particle, membrane, multi-component complex, organelle, cell, other cellular moieties, any fragment thereof, and any combination thereof.

As used herein, the term “aptamer sequence” refers to a non-naturally occurring or synthetic oligonucleotide that specifically binds to a target molecule. Typically an aptamer is from 20 to 250 nucleotides. Typically an aptamer binds to its target through secondary structure rather than sequence homology.

As used herein, the term “small molecule” refers to an organic compound that has a molecular weight of no more than 900 daltons. A small molecule is capable of modulating a cellular process or is a fluorophore.

As used herein, the term “conjugation moiety” refers to a modified nucleotide comprising a functional group for use in a method of conjugation.

As used herein, the term “linear counterpart” refers to a polyribonucleotide having the same nucleotide sequence and nucleic acid modifications as a circular polyribonucleotide and having two free ends (i.e., the uncircularized version of the circularized polyribonucleotide). In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

### Circular Polyribonucleotides

Circular polyribonucleotides (circRNA) described herein are polyribonucleotides that form a continuous structure through covalent or non-covalent bonds.

The present invention described herein includes compositions comprising synthetic circRNA and methods of their use. Due to the circular structure, circRNA can have improved stability, increased half-life, reduced immunogenicity, and/or improved functionality (e.g., of a function described herein) compared to a corresponding linear RNA. In some embodiments, the circular RNA is detectable for at least 5 days after delivery of the circular RNA to a cell. In some embodiments, the circular RNA is detectable for at 6 days, 7 days, 8 days, 9 days, 10 days, 11 days, 12 days, 13 days, 14 days, 15 days, or 16 days after delivery of the circular RNA to the cell. The circular RNA can be detected using any technique known in the art.

In some embodiments, circRNA binds one or more targets. In some embodiments, a circRNA is a circular aptamer. In one embodiment, a circRNA comprises one or more binding sites that bind to one or more targets. In one embodiment, the circ RNA comprises an aptamer sequence. In one embodiment, circRNA binds both a DNA target and a protein target and e.g., mediates transcription. In another embodiment, circRNA brings together a protein complex and e.g., mediates post-translational modifications or signal transduction. In another embodiment, circRNA binds two or more different targets, such as proteins, and e.g., shuttles these proteins to the cytoplasm, or mediates degradation of one or more of the targets.

In some embodiments, circRNA binds at least one of DNA, RNA, and proteins and thereby regulates cellular processes (e.g., alter protein expression, modulate gene expression, modulate cell signaling, etc.). In some embodiments, synthetic circRNA includes binding sites for interaction with a target or at least one moiety, e.g., a binding moiety, of DNA, RNA or proteins of choice to thereby compete in binding with the endogenous counterpart.

In some embodiments, the circular RNA forms a complex that regulates the cellular process (e.g., alter protein expression, modulate gene expression, modulate cell signaling, etc.). In some embodiments, the circular RNA sensitizes a cell to a cytotoxic agent (e.g., a chemotherapeutic agent) by binding to a target (e.g., a transcription factor), which results in reduce cell viability. For example, sensitizing the cell to the cytoxic agent results in decreased cell viability after the delivery of the cytotoxic agent and the circular RNA. In some embodiments, the decreased cell viability is decreased by at least 10%, 20%, 30%, 40%, 50%, 60%, 70%, 80%, or 90%, or any percentage therein.

In some embodiments, the complex is detectable for at least 5 days after delivery of the circular RNA to cell. In some embodiments, the complex is detectable for at 6 days, 7 days, 8 days, 9 days, 10 days, 11 days, 12 days, 13 days, 14 days, 15 days, or 16 days after delivery of the circular RNA to the cell.

In one embodiment, synthetic circRNA binds and/or sequesters miRNAs. In another embodiment, synthetic circRNA binds and/or sequesters proteins. In another embodiment, synthetic circRNA binds and/or sequesters mRNA. In another embodiment, synthetic circRNA binds and/or sequesters ribosomes. In another embodiment, synthetic circRNA binds and/or sequesters circRNA. In another embodiment, synthetic circRNA binds and/or sequesters long-noncoding RNA (lncRNA) or any other non-coding RNA, e.g., miRNA, tRNA, rRNA, snoRNA, ncRNA, siRNA, long-noncoding RNA, shRNA. Besides binding and/or sequestration sites, the circRNA may include a degradation element, which will result in degradation of the bound and/or sequestered RNA and/or protein.

In one embodiment, a circRNA comprises a lncRNA or a sequence of a lncRNA, e.g., a circRNA comprises a sequence of a naturally occurring, non-circular lncRNA or a fragment thereof. In one embodiment, a lncRNA or a sequence of a lncRNA is circularized, with or without a spacer sequence, to form a synthetic circRNA.

In one embodiment, a circRNA has ribozyme activity. In one embodiment, a circRNA can be used to act as a ribozyme and cleave pathogenic or endogenous RNA, DNA, small molecules or protein. In one embodiment, a circRNA has enzymatic activity. In one embodiment, synthetic circRNA is able to specifically recognize and cleave RNA (e.g., viral RNA). In another embodiment circRNA is able to specifically recognize and cleave proteins. In another embodiment circRNA is able to specifically recognize and degrade small molecules.

In one embodiment, a circRNA is an immolating or self-cleaving or cleavable circRNA. In one embodiment, a circRNA can be used to deliver RNA, e.g., miRNA, tRNA, rRNA, snoRNA, ncRNA, siRNA, long-noncoding RNA, shRNA. In one embodiment, synthetic circRNA is made up of microRNAs separated by (1) self-cleavable elements (e.g., hammerhead, splicing element), (2) cleavage recruitment sites (e.g., ADAR), (3) a degradable linker (e.g., glycerol), (4) a chemical linker, and/or (5) a spacer sequence. In another embodiment, synthetic circRNA is made up of siRNAs separated by (1) self-cleavable elements (e.g., hammerhead, splicing element), (2) cleavage recruitment sites (e.g., ADAR), (3) a degradable linker (e.g., glycerol), (4), chemical linker, and/or (5) a spacer sequence.

In one embodiment, a circRNA is a transcriptionally/replication competent circRNA. This circRNA can encode any type of RNA. In one embodiment, a synthetic circRNA has an anti-sense miRNA and a transcriptional element. In one embodiment, after transcription, linear functional miRNAs are generated from a circRNA. In one embodiment, a circRNA is a translation incompetent circular polyribonucleotide.

In one embodiment, a circRNA has one or more of the above attributes in combination with a translating element.

In some embodiments, a circRNA comprises at least one modified nucleotide. In some embodiments, a circRNA comprises at least 10%, 20%, 30%, 40%, 50%, 60%, 70%, or 80% modified nucleotides. In some embodiments, a circRNA comprises substantially all (e.g., greater than 80%, 85%, 90%, 95%, 97%, 98%, or 99%, or about 100%) modified nucleotides. In some embodiments, a circRNA comprises modified nucleotides and a portion of unmodified contiguous nucleotides, which can be referred to as a hybrid modified circRNA. A portion of unmodified contiguous nucleotides can be an unmodified binding site configured to bind a protein, DNA, RNA, or a cell target in a hybrid modified circRNA. A portion of unmodified contiguous nucleotides can be an unmodified IRES in a hybrid modified circRNA. In other embodiments, a circRNA lacks modified nucleotides, which can be referred to as an unmodified circRNA.

### Targets

A circRNA can comprise at least one binding site for a target, e.g., for a binding moiety of a target. A circRNA can comprise at least one aptamer sequence that binds to a target. In some embodiments, the circRNA comprises one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, a target is a polypeptide, a protein, or any fragment thereof. For example, a target can be a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a tyrosine kinase, a serine/threonine kinase, a phosphatase, an aromatase, a phosphodiesterase, a cyclase, a helicase, a protease, an oxidoreductase, a reductase, a transferase, a hydrolase, a lyase, an isomerase, a glycosylase, a extracellular matrix protein, a ligase, a ubiquitin ligase, any ligase that affects post-translational modification, an ion transporter, a channel, a pore, an apoptotic protein, a cell adhesion protein, a pathogenic protein, an aberrantly expressed protein, a transcription factor, a transcription regulator, a translation protein, an epigenetic factor, an epigenetic regulator, a chromatin regulator, a chaperone, a secreted protein, a ligand, a hormone, a cytokine, a chemokine, a nuclear protein, a receptor, a transmembrane receptor, a receptor tyrosine kinase, a G-protein coupled receptor, a growth factor receptor, a nuclear receptor, a hormone receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, a proto-oncogene, an oncogene, a tumor-suppressor gene, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, mammalian, or insect cells (e.g., proteins overexpressed by the organisms). In some embodiments, a target is a polypeptide with a mutation, insertion, deletion, or polymorphism. In some embodiments, a target is a polypeptide naturally expressed by a cell (e.g., a healthy cell or a cell associated with a disease or condition). In some embodiments, a target is an antigen, such as a polypeptide used to immunize an organism or to generate an immune response in an organism, such as for antibody production.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2}, and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., IgG₁, IgG₂, IgG₃, IgG₄, IgA₁ and IgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, tri specific, tetraspecific, pentaspecific, hexaspecific, heptaspecific, or octaspecific antibodies can be generated, e.g., by recombinantly joining a combination of any two or more antigen binding agents (e.g., Fab, F(ab)₂, scFv, Fv, IgG). Multi-specific antibodies can be used to bring two or more targets into close proximity, e.g., degradation machinery and a target substrate to degrade, or a ubiquitin ligase and a substrate to ubiquitinate. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety or target molecule, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is fortuitous and is not thought to play a role in the binding of an aptamer to its cognate target. Aptamers are differentiated from naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences will differ from aptamers as a result of the relatively low binding affinity used in nature as compared to tightly binding aptamers. Aptamers are capable of specifically binding to selected partners and modulating the partner's activity or binding interactions, e.g., through binding, aptamers may block their partner's ability to function. The functional property of specific binding to a partner is an inherent property an aptamer. An aptamer can be 6-35 kDa. An aptamer can be from 20 to 250 nucleotides. An aptamer can bind its partner with micromolar to sub-nanomolar affinity, and may discriminate against closely related targets (e.g., aptamers may selectively bind related proteins from the same gene family). In some cases, an aptamer only binds one molecule. In some cases, an aptamer binds family members of a molecule of interest. An aptamer, in some cases, binds to multiple different molecules. Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides. An aptamer can be a linear ribonucleic acid (e.g., linear aptamer) comprising an aptamer sequence or a circular polyribonucleic acid comprising an aptamer sequence (e.g., a circular aptamer).

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a target is a cell. For example, a target is an intact cell, a cell treated with a compound (e.g., a drug), a fixed cell, a lysed cell, or any combination thereof. In some embodiments, a target is a single cell. In some embodiments, a target is a plurality of cells.

In some embodiments, circRNA comprises a binding site to a single target or a plurality of (e.g., two or more) targets. In one embodiment, the single circRNA comprises 2, 3, 4, 5, 6, 7, 8, 9, 10, or more different binding sites for a single target. In one embodiment, the single circRNA comprises 2, 3, 4, 5, 6, 7, 8, 9, 10, or more of the same binding sites for a single target. In one embodiment, the single circRNA comprises 2, 3, 4, 5, 6, 7, 8, 9, 10, or more different binding sites for one or more different targets. In one embodiment, two or more targets are in a sample, such as a mixture or library of targets, and the sample comprises circRNA comprising two or more binding sites that bind to the two or more targets.

In some embodiments, a single target or a plurality of (e.g., two or more) targets have a plurality of binding moieties. In one embodiment, the single target may have 2, 3, 4, 5, 6, 7, 8, 9, 10, or more binding moieties. In one embodiment, two or more targets are in a sample, such as a mixture or library of targets, and the sample comprises two or more binding moieties. In some embodiments, a single target or a plurality of targets comprise a plurality of different binding moieties. For example, a plurality may include at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties.

A target can comprise a plurality of binding moieties comprising at least 2 different binding moieties. For example, a binding moiety can comprise a plurality of binding moieties comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different binding moieties.

### Binding Sites and Binding Moieties

In some instances, a circRNA comprises one binding site. A binding site can comprise an aptamer sequence. In some instances, a circRNA comprises at least two binding sites. For example, a circRNA can comprise 2, 3, 4, 5, 6, 7, 8, 9, 10, 11, 12, 13, 14, 15, 16, 17, 18, 19, 20, or more binding sites. In some embodiments, circRNA described herein is a molecular scaffold that binds one or more targets, or one or more binding moieties of one or more targets. Each target may be, but is not limited to, a different or the same nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, cellular moieties, any fragments thereof, and any combination thereof. In some embodiments, the one or more binding sites binds to the same target. In some embodiments, the one or more binding sites bind to one or more binding moieties of the same target. In some embodiments, the one or more binding sites bind to one or more different targets. In some embodiments, the one or more binding sites bind to one or more binding moieties of different targets. In some embodiments, a circRNA acts as a scaffold for one or more binding one or more targets. In some embodiments, a circRNA acts as a scaffold for one or more binding moieties of one or more targets. In some embodiments, a circRNA modulates cellular processes by specifically binding to one or more one or more targets. In some embodiments, a circRNA modulates cellular processes by specifically binding to one or more binding moieties of one or more targets. In some embodiments, a circRNA modulates cellular processes by specifically binding to one or more targets. In some embodiments, a circRNA described herein includes binding sites for one or more specific targets of interest. In some embodiments, circRNA includes multiple binding sites or a combination of binding sites for each target of interest. In some embodiments, circRNA includes multiple binding sites or a combination of binding sites for each binding moiety of interest. For example, a circRNA can include one or more binding sites for a polypeptide target. In some embodiments, a circRNA includes one or more binding sites for a polynucleotide target, such as a DNA or RNA, an mRNA target, an rRNA target, a tRNA target, or a genomic DNA target.

In some instances, a circRNA comprises a binding site for a single-stranded DNA. In some instances, a circRNA comprises a binding site for double-stranded DNA. In some instances, a circRNA comprises a binding site for an antibody. In some instances, a circRNA comprises a binding site for a virus particle. In some instances, a circRNA comprises a binding site for a small molecule. In some instances, a circRNA comprises a binding site that binds in or on a cell. In some instances, a circRNA comprises a binding site for a RNA-DNA hybrid. In some instances, a circRNA comprises a binding site for a methylated polynucleotide. In some instances, a circRNA comprises a binding site for an unmethylated polynucleotide. In some instances, a circRNA comprises a binding site for an aptamer. In some instances, a circRNA comprises a binding site for a polypeptide. In some instances, a circRNA comprises a binding site for a polypeptide, a protein, a protein fragment, a tagged protein, an antibody, an antibody fragment, a small molecule, a virus particle (e.g., a virus particle comprising a transmembrane protein), or a cell. In some instances, a circRNA comprises a binding site for a binding moiety on a single-stranded DNA. In some instances, a circRNA comprises a binding site for a binding moiety on a double-stranded DNA. In some instances, a circRNA comprises a binding site for a binding moiety on an antibody. In some instances, a circRNA comprises a binding site for a binding moiety on a virus particle. In some instances, a circRNA comprises a binding site for a binding moiety on a small molecule. In some instances, a circRNA comprises a binding site for a binding moiety in or on a cell. In some instances, a circRNA comprises a binding site for a binding moiety on a RNA-DNA hybrid. In some instances, a circRNA comprises a binding site for a binding moiety on a methylated polynucleotide. In some instances, a circRNA comprises a binding site for a binding moiety on an unmethylated polynucleotide. In some instances, a circRNA comprises a binding site for a binding moiety on an aptamer. In some instances, a circRNA comprises a binding site for a binding moiety on a polypeptide. In some instances, a circRNA comprises a binding site for a binding moiety on a polypeptide, a protein, a protein fragment, a tagged protein, an antibody, an antibody fragment, a small molecule, a virus particle (e.g., a virus particle comprising a transmembrane protein), or a cell.

In some instances, a binding site binds to a portion of a target comprising at least two amide bonds. In some instances, a binding site does not bind to a portion of a target comprising a phosphodiester linkage. In some instances, a portion of the target is not DNA or RNA. In some instances, a binding moiety comprises at least two amide bonds. In some instances, a binding moiety does not comprise a phosphodiester linkage. In some instances, a binding moiety is not DNA or RNA.

The circRNAs provided herein can include one or more binding sites for binding moieties on a complex. The circRNAs provided herein can include one or more binding sites for targets to form a complex. For example, the circRNAs provided herein can act as a scaffold to form a complex between a circRNA and a target. In some embodiments, a circRNA forms a complex with a single target. In some embodiments, a circRNA forms a complex with two targets. In some embodiments, a circRNA forms a complex with three targets. In some embodiments, a circRNA forms a complex with four targets. In some embodiments, a circRNA forms a complex with five or more targets. In some embodiments, a circRNA forms a complex with a complex of two or more targets. In some embodiments, a circRNA forms a complex with a complex of three or more targets. In some embodiments, two or more circRNAs form a complex with a single target. In some embodiments, two or more circRNAs form a complex with two or more targets. In some embodiments, a first circRNA forms a complex with a first binding moiety of a first target and a second different binding moiety of a second target. In some embodiments, a first circRNA forms a complex with a first binding moiety of a first target and a second circRNA forms a complex with a second binding moiety of a second target.

In some embodiments, a circRNA can include a binding site for one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some embodiments, a circRNA can include a binding site for one or more binding moieties on one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some instances, a binding site binds to a polypeptide, protein, or fragment thereof. In some embodiments, a binding site binds to a domain, a fragment, an epitope, a region, or a portion of a polypeptide, protein, or fragment thereof of a target. For example, a binding site binds to a domain, a fragment, an epitope, a region, or a portion of an isolated polypeptide, a polypeptide of a cell, a purified polypeptide, or a recombinant polypeptide. For example, a binding site binds to a domain, a fragment, an epitope, a region, or a portion of an antibody or fragment thereof. For example, a binding site binds to a domain, a fragment, an epitope, a region, or a portion of a transcription factor. For example, a binding site binds to a domain, a fragment, an epitope, a region, or a portion of a receptor. For example, a binding site binds to a domain, a fragment, an epitope, a region, or a portion of a transmembrane receptor. Binding sites may bind to a domain, a fragment, an epitope, a region, or a portion of isolated, purified, and/or recombinant polypeptides. Binding sites can bind to a domain, a fragment, an epitope, a region, or a portion of a mixture of analytes (e.g., a lysate). For example, a binding site binds to a domain, a fragment, an epitope, a region, or a portion of from a plurality of cells or from a lysate of a single cell. A binding site can bind to a binding moiety of a target. In some instances, a binding moiety is on a polypeptide, protein, or fragment thereof. In some embodiments, a binding moiety comprises a domain, a fragment, an epitope, a region, or a portion of a polypeptide, protein, or fragment thereof. For example, a binding moiety comprises a domain, a fragment, an epitope, a region, or a portion of an isolated polypeptide, a polypeptide of a cell, a purified polypeptide, or a recombinant polypeptide. For example, a binding moiety comprises a domain, a fragment, an epitope, a region, or a portion of an antibody or fragment thereof. For example, a binding moiety comprises a domain, a fragment, an epitope, a region, or a portion of a transcription factor. For example, a binding moiety comprises a domain, a fragment, an epitope, a region, or a portion of a receptor. For example, a binding moiety comprises a domain, a fragment, an epitope, a region, or a portion of a transmembrane receptor. Binding moieties may be on or comprise a domain, a fragment, an epitope, a region, or a portion of isolated, purified, and/or recombinant polypeptides. Binding moieties include binding moieties on or a domain, a fragment, an epitope, a region, or a portion of a mixture of analytes (e.g., a lysate). For example, binding moieties are on or comprise a domain, a fragment, an epitope, a region, or a portion of from a plurality of cells or from a lysate of a single cell.

In some instances, a binding site binds to a domain, a fragment, an epitope, a region, or a portion of a chemical compound (e.g., small molecule). For example, a binding binds to a domain, a fragment, an epitope, a region, or a portion of a drug. For example, a binding site binds to a domain, a fragment, an epitope, a region, or a portion of a compound. For example, a binding moiety binds to a domain, a fragment, an epitope, a region, or a portion of an organic compound. In some instances, a binding site binds to a domain, a fragment, an epitope, a region, or a portion of a small molecule with a molecular weight of 900 Daltons or less. In some instances, a binding site binds to a domain, a fragment, an epitope, a region, or a portion of a small molecule with a molecular weight of 500 Daltons or more. The portion the small molecule that the binding site binds to may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference. A binding site can bind to a binding moiety of a small molecule. In some instances, a binding moiety is on or comprises a domain, a fragment, an epitope, a region, or a portion of a small molecule. For example, a binding moiety is on or comprises a domain, a fragment, an epitope, a region, or a portion of a drug. For example, a binding moiety is on or comprises a domain, a fragment, an epitope, a region, or a portion of a compound. For example, a binding moiety is on or comprises a domain, a fragment, an epitope, a region, or a portion of an organic compound. In some instances, a binding moiety is on or comprises a domain, a fragment, an epitope, a region, or a portion of a small molecule with a molecular weight of 900 Daltons or less. In some instances, a binding moiety is on or comprises a domain, a fragment, an epitope, a region, or a portion of a small molecule with a molecular weight of 500 Daltons or more. Binding moieties may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A binding site can bind to a domain, a fragment, an epitope, a region, or a portion of a member of a specific binding pair (e.g., a ligand). A binding site can bind to a domain, a fragment, an epitope, a region, or a portion of monovalent (monoepitopic) or polyvalent (polyepitopic). A binding site can bind to an antigenic or haptenic portion of a target. A binding site can bind to a domain, a fragment, an epitope, a region, or a portion of a single molecule or a plurality of molecules that share at least one common epitope or determinant site. A binding site can bind to a domain, a fragment, an epitope, a region, or a portion of a part of a cell (e.g., a bacteria cell, a plant cell, or an animal cell). A binding site can bind to a target that is in a natural environment (e.g., tissue), a cultured cell, or a microorganism (e.g., a bacterium, fungus, protozoan, or virus), or a lysed cell. A binding site can bind to a portion of a target that is modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group. A binding site can bind to a binding moiety of a member of a specific binding pair. A binding moiety can be on or comprise a domain, a fragment, an epitope, a region, or a portion of a member of a specific binding pair (e.g., a ligand). A binding moiety can be on or comprise a domain, a fragment, an epitope, a region, or a portion of monovalent (monoepitopic) or polyvalent (polyepitopic). A binding moiety can be antigenic or haptenic. A binding moiety can be on or comprise a domain, a fragment, an epitope, a region, or a portion of a single molecule or a plurality of molecules that share at least one common epitope or determinant site. A binding moiety can be on or comprise a domain, a fragment, an epitope, a region, or a portion of a part of a cell (e.g., a bacteria cell, a plant cell, or an animal cell). A binding moiety can be either in a natural environment (e.g., tissue), a cultured cell, or a microorganism (e.g., a bacterium, fungus, protozoan, or virus), or a lysed cell. A binding moiety can be modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

In some instances, a binding site binds to a domain, a fragment, an epitope, a region, or a portion of a molecule found in a sample from a host. A binding site can bind to a binding moeity of a molecule found in a sample from a host. In some instances, a binding moiety is on or comprises a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render a binding moiety more readily detectible. Samples include a quantity of a substance from a living thing or formerly living things. A sample can be natural, recombinant, synthetic, or not naturally occurring. A binding site can bind to any of the above that is expressed from a cell naturally or recombinantly, in a cell lysate or cell culture medium, an in vitro translated sample, or an immunoprecipitation from a sample (e.g., a cell lysate). A binding moiety can be any of the above that is expressed from a cell naturally or recombinantly, in a cell lysate or cell culture medium, an in vitro translated sample, or an immunoprecipitation from a sample (e.g., a cell lysate).

In some instances, a binding site binds to a target expressed in a cell-free system or in vitro. For example, a binding site binds to a target in a cell extract. In some instances, a binding site binds to a target in a cell extract with a DNA template, and reagents for transcription and translation. A binding site can bind to a binding moiety of a a target expressed in a cell-free system or in vitro. In some instances, a binding moiety of a target is expressed in a cell-free system or in vitro. For example, a binding moiety of a target is in a cell extract. In some instances, a binding moiety of a target is in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and OP-microintaglio printing, and pMAC-protein microarray copying (See Kilb et al., Eng. Life Sci. 2014, 14, 352-364).

In some instances, a binding site binds to a target that is synthesized in situ (e.g., on a solid substrate of an array) from a DNA template. A binding site can bind to binding moiety of a target that is synthesized in situ. In some instances, a binding moiety of a target is synthesized in situ (e.g., on a solid substrate of an array) from a DNA template. In some instances, a plurality of binding moieties is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, a binding site binds to a nucleic acid target comprising a span of at least 6 nucleotides, for example, least 8, 9, 10, 12, 15, 20, 25, 30, 40, 50, or 100 nucleotides. In some instances, a binding site binds to a protein target comprising a contiguous stretch of nucleotides. In some instances, a binding site binds to a protein target comprising a non-contiguous stretch of nucleotides. In some instances, a binding site binds to a nucleic acid target comprising a site of a mutation or functional mutation, including a deletion, addition, swap, or truncation of the nucleotides in a nucleic acid sequence. A binding site can bind to a binding moiety of a nucleic acid target. In some instances, a binding moiety of a nucleic acid target comprises a span of at least 6 nucleotides, for example, least 8, 9, 10, 12, 15, 20, 25, 30, 40, 50, or 100 nucleotides. In some instances, a binding moiety of a protein target comprises a contiguous stretch of nucleotides. In some instances, a binding moiety of a protein target comprises a non-contiguous stretch of nucleotides. In some instances, a binding moiety of a nucleic acid target comprises a site of a mutation or functional mutation, including a deletion, addition, swap, or truncation of the nucleotides in a nucleic acid sequence.

In some instances, a binding site binds to a protein target comprising a span of at least 6 amino acids, for example, least 8, 9, 10, 12, 15, 20, 25, 30, 40, 50, or 100 amino acids. In some instances, a binding site binds to a protein target comprising a contiguous stretch of amino acids. In some instances, a binding site binds to a protein target comprising a non-contiguous stretch of amino acids. In some instances, a binding site binds to a protein target comprising a site of a mutation or functional mutation, including a deletion, addition, swap, or truncation of the amino acids in a polypeptide sequence. A binding site can bind to a binding moiety of a protein target. In some instances, a binding moiety of a protein target comprises a span of at least 6 amino acids, for example, least 8, 9, 10, 12, 15, 20, 25, 30, 40, 50, or 100 amino acids. In some instances, a binding moiety of a protein target comprises a contiguous stretch of amino acids. In some instances, a binding moiety of a protein target comprises a non-contiguous stretch of amino acids. In some instances, a binding moiety of a protein target comprises a site of a mutation or functional mutation, including a deletion, addition, swap, or truncation of the amino acids in a polypeptide sequence.

In some embodiments, a binding site binds to a domain, a fragment, an epitope, a region, or a portion of a membrane bound protein. A binding site can bind to a binding moiety of a membrane bound protein. In some embodiments, a binding moiety is on or comprises a domain, a fragment, an epitope, a region, or a portion of a membrane bound protein. Exemplary membrane bound proteins include, but are not limited to, GPCRs (e.g., adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), non-excitable and excitable channels, receptor tyrosine kinases, receptor serine/threonine kinases, receptor guanylate cyclases, growth factor and hormone receptors (e.g., epidermal growth factor (EGF) receptor), and others. The binding site can bind to a domain, a fragment, an epitope, a region, or a portion of a mutant or modified variants of membrane-bound proteins. The binding site can bind to a binding moiety of a mutant or modified variant of membrane-bound protein. The binding moiety may also be on or comprise a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

A binding site binds to, for example, a domain, a fragment, an epitope, a region, or a portion of a ubiquitin ligase. A binding site binds to, for example, a domain, a fragment, an epitope, a region, or a portion of a ubiquitin adaptor, proteasome adaptor, or proteasome protein. A binding site binds to, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof. In some instance, the binding site binds to a binding moiety. A binding moiety can comprise, for example, a domain, a fragment, an epitope, a region, or a portion of a ubiquitin ligase. A binding moiety can comprise, for example, a domain, a fragment, an epitope, a region, or a portion of a ubiquitin adaptor, proteasome adaptor, or proteasome protein. A binding moiety can comprise, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof.

A binding site binds to, for example, a domain, a fragment, an epitope, a region, or a portion of a protein associated with a disease or condition. A binding site binds to, for example, a domain, a fragment, an epitope, a region, or a portion of a proto-oncogene. A binding site binds to, for example, a domain, a fragment, an epitope, a region, or a portion of an oncogene. A binding site binds to, for example, a domain, a fragment, an epitope, a region, or a portion of a tumor suppressor gene. A binding site binds to, for example, a domain, a fragment, an epitope, a region, or a portion of an inflammatory gene (e.g., a cytokine). A binding site can bind to a binding moiety. A binding moiety can comprise, for example, a domain, a fragment, an epitope, a region, or a portion of a protein associated with a disease or condition. A binding moiety can comprise, for example, a domain, a fragment, an epitope, a region, or a portion of a proto-oncogene. A binding moiety can comprise, for example, a domain, a fragment, an epitope, a region, or a portion of an oncogene. A binding moiety can comprise, for example, a domain, a fragment, an epitope, a region, or a portion of a tumor suppressor gene. A binding moiety can comprise, for example, a domain, a fragment, an epitope, a region, or a portion of an inflammatory gene (e.g., a cytokine).

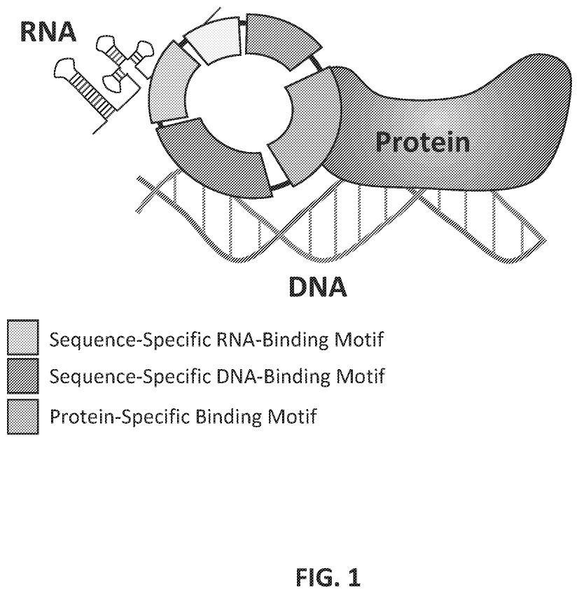

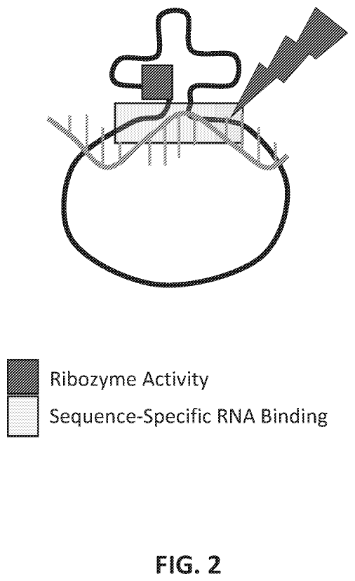

FIG. 1 shows an example of a circular polyribonucleotide with a sequence-specific RNA-binding motif, sequence-specific DNA-binding motif, and protein-specific binding motif. In some embodiments, circRNA can include other binding motifs for binding other intracellular molecules. Non-limiting examples of circRNA applications are listed in TABLE 1.

TABLE 1

| Process | MOA (example) |
|---|---|
| Directed Transcription | DNA-circRNA-Protein (pol, TF) |
| Epigenetic Remodeling | DNA-circRNA-Protein (SWI/SNF) |
| Transcriptional interference | circRNA-DNA |
| Translational interference | circRNA-mRNA or ribosome |
| Protein interaction inhibitor | circRNA-Protein |
| Protein Degradation | Protein-circRNA-Protein (ubiq) |
| RNA Degradation | RNA-circRNA-RNA (RNAse to RNA) |
| DNA Degradation | DNA-circRNA-Protein (DNA to DNAse) |
| Artificial Receptor | Cell Surface-circRNA-Substrate |
| Protein Translocation | Protein-circRNA-Protein/RNA |
| Cellular Fusion | Cell Surface-circRNA-Cell Surface |
| Complex Disassembly | Protein-circRNA-Protein/RNA |
| Receptor inhibition | Protein-circRNA-Substrate |
| Signal Transduction | Protein-circRNA-Protein (caspase) |
| Multi-Enzyme Acceleration | Multiple Enzyems-circRNA |
| Induction of receptor | circRNA-receptor |

RNA Binding Sites

In some embodiments, the circular polyribonucleotide comprises one or more RNA binding sites. In some embodiments, the circular polyribonucleotide includes RNA binding sites that modify expression of an endogenous gene and/or an exogenous gene. In some embodiments, the RNA binding site modulates expression of a host gene. The RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises an aptamer sequence that binds to an RNA. The aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the aptamer sequence can bind to the RNA. The circular RNA can form a complex with the RNA by binding of the aptamer sequence to the RNA.

In some embodiments, the RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. RNA binding sites are well-known to persons of ordinary skill in the art.

Certain RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates.

In some embodiments, the RNA binding site comprises an siRNA or an shRNA. siRNA and shRNA resemble intermediates in the processing pathway of the endogenous miRNA genes. In some embodiments, siRNA can function as miRNA and vice versa. MicroRNA, like siRNA, can use RISC to downregulate target genes, but unlike siRNA, most animal miRNA do not cleave the mRNA. Instead, miRNA reduce protein output through translational suppression or polyA removal and mRNA degradation. Known miRNA binding sites are within mRNA 3′-UTRs; miRNA seem to target sites with near-perfect complementarity to nucleotides 2-8 from the miRNA's 5′ end. This region is known as the seed region. Because siRNA and miRNA are interchangeable, exogenous siRNA can downregulate mRNA with seed complementarity to the siRNA. Multiple target sites within a 3′-UTR can give stronger downregulation.

MicroRNA (miRNA) are short noncoding RNA that bind to the 3′-UTR of nucleic acid molecules and down-regulate gene expression either by reducing nucleic acid molecule stability or by inhibiting translation. The circular polyribonucleotide can comprise one or more miRNA target sequences, miRNA sequences, or miRNA seeds. Such sequences can correspond to any miRNA.

A miRNA sequence comprises a “seed” region, i.e., a sequence in the region of positions 2-8 of the mature miRNA, which sequence has Watson-Crick complementarity to the miRNA target sequence. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA target is flanked by an adenine (A) opposed to miRNA position 1. In some embodiments, a miRNA seed can comprise 6 nucleotides (e.g., nucleotides 2-7 of the mature miRNA), wherein the seed-complementary site in the corresponding miRNA target is flanked by an adenine (A) opposed to miRNA at position 1.

The bases of the miRNA seed can be substantially complementary with the target sequence. By engineering miRNA target sequences into the circular polyribonucleotide, the circular polyribonucleotide can evade or be detected by the host's immune system, have modulated degradation, or modulated translation. This process can reduce the hazard of off target effects upon circular polyribonucleotide delivery.

The circular polyribonucleotide can include an miRNA sequence identical to about 5 to about 25 contiguous nucleotides of a target gene. In some embodiments, the miRNA sequence targets a mRNA and commences with the dinucleotide AA, comprises a GC-content of about 30%-70%, about 30%-60%, about 40%-60%, or about 45%-55%, and does not have a high percentage identity to any nucleotide sequence other than the target in the genome of the mammal in which it is to be introduced, for example, as determined by standard BLAST search.

Conversely, miRNA binding sites can be engineered out of (i.e., removed from) the circular polyribonucleotide to modulate protein expression in specific tissues. Regulation of expression in multiple tissues can be accomplished through introduction or removal or one or several miRNA binding sites.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MiRNA can also regulate complex biological processes, such as angiogenesis (miR-132). In the circular polyribonucleotides described herein, binding sites for miRNA that are involved in such processes can be removed or introduced, in order to tailor the expression from the circular polyribonucleotide to biologically relevant cell types or to the context of relevant biological processes. In some embodiments, the miRNA binding site includes, e.g., miR-7.

Through an understanding of the expression patterns of miRNA in different cell types, the circular polyribonucleotide described herein can be engineered for more targeted expression in specific cell types or only under specific biological conditions. Through introduction of tissue-specific miRNA binding sites, the circular polyribonucleotide can be designed for optimal protein expression in a tissue or in the context of a biological condition.

In addition, miRNA seed sites can be incorporated into the circular polyribonucleotide to modulate expression in certain cells which results in a biological improvement. An example of this is incorporation of miR-142 sites. Incorporation of miR-142 sites into the circular polyribonucleotide described herein can modulate expression in hematopoietic cells, but also reduce or abolish immune responses to a protein encoded in the circular polyribonucleotide.

In some embodiments, the circular polyribonucleotide comprises at least one miRNA, e.g., 2, 3, 4, 5, 6, or more. In some embodiments, the circular polyribonucleotide comprises an miRNA having at least about 75%, about 80%, about 85%, about 90%, about 95%, about 96%, about 97%, about 98%, about 99%, or 100% nucleotide sequence identity to any one of the nucleotide sequences or a sequence that is complementary to a target sequence.

Lists of known miRNA sequences can be found in databases maintained by research organizations, for example, Wellcome Trust Sanger Institute, Penn Center for Bioinformatics, Memorial Sloan Kettering Cancer Center, and European Molecule Biology Laboratory. RNAi molecules can be readily designed and produced by technologies known in the art. In addition, computational tools can be used to determine effective and specific sequence motifs.

In some embodiments, a circular polyribonucleotide comprises a long non-coding RNA. Long non-coding RNA (lncRNA) include non-protein coding transcripts longer than 100 nucleotides. The longer length distinguishes lncRNA from small regulatory RNA, such as miRNA, siRNA, and other short RNA. In general, the majority (˜78%) of lncRNA are characterized as tissue-specific. Divergent lncRNA that are transcribed in the opposite direction to nearby protein-coding genes (comprise a significant proportion ˜20% of total lncRNA in mammalian genomes) can regulate the transcription of the nearby gene.

The length of the RNA binding site may be between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more nucleotides. The degree of identity of the RNA binding site to a target of interest can be at least 75%, at least 80%, at least 85%, at least 90%, or at least 95%.

In some embodiments, the circular polyribonucleotide includes one or more large intergenic non-coding RNA (lincRNA) binding sites. LincRNA make up most of the long non-coding RNA. LincRNA are non-coding transcripts and, in some embodiments, are more than about 200 nucleotides long. In some embodiments, lincRNA have an exon-intron-exon structure, similar to protein-coding genes, but do not encompass open-reading frames and do not code for proteins. LincRNA expression can be strikingly tissue-specific compared to coding genes. LincRNA are typically co-expressed with their neighboring genes to a similar extent to that of pairs of neighboring protein-coding genes. In some embodiments, the circular polyribonucleotide comprises a circularized lincRNA.

In some embodiments, the circular polyribonucleotides disclosed herein include one or more lincRNA, for example, FIRRE, LINC00969, PVT1, LINC01608, JPX, LINC01572, LINC00355, C1orf132, C3orf35, RP11-734, LINC01608, CC-499B15.5, CASC15, LINC00937, and RP11-191.

Lists of known lincRNA and lncRNA sequences can be found in databases maintained by research organizations, for example, Institute of Genomics and Integrative Biology, Diamantina Institute at the University of Queensland, Ghent University, and Sun Yat-sen University. LincRNA and lncRNA molecules can be readily designed and produced by technologies known in the art. In addition, computational tools can be used to determine effective and specific sequence motifs.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

In some embodiments, the circular polyribonucleotide comprises a RNA binding site that has an RNA or RNA-like structure typically between about 5-5000 base pairs (depending on the specific RNA structure, e.g., miRNA 5-30 bps, lncRNA 200-500 bps) and has a nucleobase sequence identical (complementary) or nearly identical (substantially complementary) to a coding sequence in an expressed target gene within the cell.

DNA Binding Sites

In some embodiments, the circular polyribonucleotide comprises a DNA binding site, such as a sequence for a guide RNA (gRNA). In some embodiments, the circular polyribonucleotide comprises a guide RNA or a complement to a gRNA sequence. A gRNA short synthetic RNA composed of a “scaffold” sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises an aptamer sequence that can bind to DNA. The secondary structure of the aptamer sequence can bind to DNA. In some embodiments, the circular polyribonucleotide forms a complex with the DNA by binding of the aptamer sequence to the DNA.

In some embodiments, the circular polyribonucleotide includes sequences that bind a major groove of in duplex DNA. In one such instance, the specificity and stability of a triplex structure created by the circular polyribonucleotide and duplex DNA is afforded via Hoogsteen hydrogen bonds, which are different from those formed in classical Watson-Crick base pairing in duplex DNA. In one instance, the circular polyribonucleotide binds to the purine-rich strand of a target duplex through the major groove.

In some embodiments, triplex formation occurs in two motifs, distinguished by the orientation of the circular polyribonucleotide with respect to the purine-rich strand of the target duplex. In some instances, polypyrimidine sequence stretches in a circular polyribonucleotides bind to the polypurine sequence stretches of a duplex DNA via Hoogsteen hydrogen bonding in a parallel fashion (i.e., in the same 5′ to 3′, orientation as the purine-rich strand of the duplex), whereas the polypurine stretches (R) bind in an antiparallel fashion to the purine strand of the duplex via reverse-Hoogsteen hydrogen bonds. In the antiparallel, a purine motif comprises triplets of G:G-C, A:A-T, or T:A-T; whereas in the parallel, a pyrimidine motif comprises canonical triples of C+:G-C or T:A-T triplets (where C+ represents a protonated cytosine on the N3 position). Antiparallel GA and GT sequences in a circular polyribonucleotide may form stable triplexes at neutral pH, while parallel CT sequences in a circular polyribonucleotide may bind at acidic pH. N3 on cytosine in the circular polyribonucleotide may be protonated. Substitution of C with 5-methyl-C may permit binding of CT sequences in the circular polyribonucleotide at physiological pH as 5-methyl-C has a higher pK than does cytosine. For both purine and pyrimidine motifs, contiguous homopurine-homopyrimidine sequence stretches of at least 10 base pairs aid circular polyribonucleotide binding to duplex DNA, since shorter triplexes may be unstable under physiological conditions, and interruptions in sequences can destabilize the triplex structure. In some embodiments, the DNA duplex target for triplex formation includes consecutive purine bases in one strand. In some embodiments, a target for triplex formation comprises a homopurine sequence in one strand of the DNA duplex and a homopyrimidine sequence in the complementary strand.

In some embodiments, a triplex comprising a circular polyribonucleotide is a stable structure. In some embodiments, a triplex comprising a circular polyribonucleotide exhibits an increased half-life, e.g., increased by about 5%, 10%, 15%, 20%, 25%, 30%, 35%, 40%, 45%, 50%, or greater, e.g., persistence for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between.

Protein Binding Sites

In some embodiments, the circular polyribonucleotide includes one or more protein binding sites. In some embodiments, a protein binding site comprises an aptamer sequence. In one embodiment, the circular polyribonucleotide includes a protein binding site to reduce an immune response from the host as compared to the response triggered by a reference compound, e.g., a circular polyribonucleotide lacking the protein binding site, e.g., linear RNA.

In some embodiments, circular polyribonucleotides disclosed herein include one or more protein binding sites to bind a protein, e.g., a ribosome. By engineering protein binding sites, e.g., ribosome binding sites, into the circular polyribonucleotide, the circular polyribonucleotide can evade or have reduced detection by the host's immune system, have modulated degradation, or modulated translation.

In some embodiments, the circular polyribonucleotide comprises at least one immunoprotein binding site, for example, to mask the circular polyribonucleotide from components of the host's immune system, e.g., evade CTL responses. In some embodiments, the immunoprotein binding site is a nucleotide sequence that binds to an immunoprotein and aids in masking the circular polyribonucleotide as non-endogenous.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) or translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

In some embodiments, circular polyribonucleotides disclosed herein comprise a protein binding sequence that binds to a protein. In some embodiments, the protein binding sequence targets or localizes a circular polyribonucleotide to a specific target. In some embodiments, the protein binding sequence specifically binds an arginine-rich region of a protein.

In some embodiments, circular polyribonucleotides disclosed herein include one or more protein binding sites that each bind a target protein, e.g., acting as a scaffold to bring two or more proteins in close proximity. In some embodiments, circular polynucleotides disclosed herein comprise two protein binding sites that each bind a target protein, thereby bringing the target proteins into close proximity. In some embodiments, circular polynucleotides disclosed herein comprise three protein binding sites that each bind a target protein, thereby bringing the three target proteins into close proximity. In some embodiments, circular polynucleotides disclosed herein comprise four protein binding sites that each bind a target protein, thereby bringing the four target proteins into close proximity. In some embodiments, circular polynucleotides disclosed herein comprise five or more protein binding sites that each bind a target protein, thereby bringing five or more target proteins into close proximity. In some embodiments, the target proteins are the same. In some embodiments, the target proteins are different. In some embodiments, bringing target proteins into close proximity promotes formation of a protein complex. For example, a circular polyribonucleotide of the disclosure can act as a scaffold to promote the formation of a complex comprising one, two, three, four, five, six, seven, eight, nine, or ten target proteins, or more. In some embodiments, bringing two or more target proteins into close proximity promotes interaction of the two or more target proteins. In some embodiments, bringing two or more target proteins into close proximity modulates, promotes, or inhibits of an enzymatic reaction. In some embodiments, bringing two or more target proteins into close proximity modulates, promotes, or inhibits a signal transduction pathway.

In some embodiments, the protein binding site includes, but is not limited to, a binding site to the protein, such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, p53, PCBP2, POLR2A, PRPF8, PTBP1, RBFOX1, RBFOX2, RBFOX3, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, a protein binding site is a nucleic acid sequence that binds to a protein, e.g., a sequence that can bind a transcription factor, enhancer, repressor, polymerase, nuclease, histone, or any other protein that binds DNA. In some embodiments, a protein binding site is an aptamer sequence that binds to a protein. In some embodiments, the secondary structure of the aptamer sequence binds the protein. In some embodiments, the circular RNA forms a complex with the protein by binding of the aptamer sequence to the protein.

In some embodiments, a circular RNA is conjugated to a small molecule or a part thereof, wherein the small molecule or part thereof binds to a target such as a protein. A small molecule can be conjugated to a circular RNA via a modified nucleotide, e.g., by click chemistry. Examples of small molecules that can bind to proteins include, but are not limited to 4-hydroxytamoxifen (4-OHT), AC220, Afatinib, an aminopyrazole analog, an AR antagonist, BI-7273, Bosutinib, Ceritinib, Chloroalkane, Dasatinib, Foretinib, Gefitinib, a HIF-la-derived (R)-hydroxyproline, HJB97, a hydroxyproline-based ligand, IACS-7e, Ibrutinib, an ibrutinib derivative, JQ1, Lapatinib, an LCL161 derivative, Lenalidomide, a nutlin small molecule, OTX015, a PDE4 inhibitor, Pomalidomide, a ripk2 inhibitor, RN486, Sirt2 inhibitor 3b, SNS-032, Steel factor, a TBK1 inhibitor, Thalidomide, a thalidomide derivative, a Thiazolidinedione-based ligand, a VH032 derivative, VHL ligand 2, VHL-1, VL-269, and derivatives thereof.

In some embodiments, a circular RNA is conjugated to more than one small molecule, for instance, 2, 3, 4, 5, 6, 7, 8, 9, 10, or more small molecules. In some embodiments, a circular RNA is conjugated to more than one different small molecules, for instance, 2, 3, 4, 5, 6, 7, 8, 9, 10, or more different small molecules. In some embodiments, the more than one small molecule conjugated to the circular RNA are configured to recruit their respective target proteins into proximity, which can lead to interaction between the target proteins, and/or other molecular and cellular changes. For instance, a circular RNA can be conjugated to both JQ1 and thalidomide, or derivative thereof, which can thus recruit a target protein of JQ1, e.g., BET family proteins, and a target protein of thalidomide, e.g., E3 ligase. In some cases, the circular RNA conjugated with JQ1 and thalidomide recruits a BET family protein via JQ1, or derivative thereof, tags the BET family protein with ubiquitin by E3 ligase that is recruited through thalidomide or derivative thereof, and thus leads to degradation of the tagged BET family protein.

Other Binding Sites

In some embodiments, the circular polyribonucleotide comprises one or more binding sites to a non-RNA or non-DNA target. In some embodiments, the binding site can be one of a small molecule, an aptamer, a lipid, a carbohydrate, a virus particle, a membrane, a multi-component complex, a cell, a cellular moiety, or any fragment thereof binding site. In some embodiments, the circular polyribonucleotide comprises one or more binding sites to a lipid. In some embodiments, the circular polyribonucleotide comprises one or more binding sites to a carbohydrate. In some embodiments, the circular polyribonucleotide comprises one or more binding sites to a carbohydrate. In some embodiments, the circular polyribonucleotide comprises one or more binding sites to a membrane. In some embodiments, the circular polyribonucleotide comprises one or more binding sites to a multi-component complex, e.g., ribosome, nucleosome, transcription machinery, etc.

In some embodiments, the circular polyribonucleotide comprises an aptamer sequence. The aptamer sequence can bind to any target as described herein (e.g., a nucleic acid molecule, a small molecule, a protein, a carbohydrate, a lipid, etc.). The aptamer sequence has a secondary structure that can bind the target. In some embodiments, the aptamer sequence has a tertiary structure that can bind the target. In some embodiments, the aptamer sequence has a quaternary structure that can bind the target. The circular polyribonucleotide can bind to the target via the aptamer sequence to form a complex. In some embodiments, the complex is detectable for at least 5 days. In some embodiments, the complex is detectable for at least 2 days, 3, days, 4 days, 5 days, 6 days, 7 days, 8 days, 9 days, 10 days, 11 days, 12 days, 13 days, 14 days, 15 days, 16 days.

### Sequestration

In some embodiments, circRNA described herein sequesters a target, e.g., DNA, RNA, proteins, and other cellular components to regulate cellular processes. CircRNA with binding sites for a target of interest can compete with binding of the target with an endogenous binding partner. In some embodiments, circRNA described herein sequesters miRNA. In some embodiments, circRNA described herein sequesters mRNA. In some embodiments, circRNA described herein sequesters proteins. In some embodiments, circRNA described herein sequesters ribosomes. In some embodiments, circRNA described herein sequesters other circRNA. In some embodiments, circRNA described herein sequesters non-coding RNA, lncRNA, miRNA, tRNA, rRNA, snoRNA, ncRNA, siRNA, or shRNA. In some embodiments, circRNA described herein includes a degradation element that degrades a sequestered target, e.g., DNA, RNA, protein, or other cellular component bound to the circRNA. Non-limiting examples of circRNA sequestration applications are listed in TABLE 2.

TABLE 2

| Process | MOA (example) |
|---|---|
| Transcriptional interference | circRNA-DNA |
| Translational interference | circRNA-mRNA or ribosome |
| Protein interaction inhibitor | circRNA-Protein |
| microRNA sequester | circRNA-RNA (antisense) |
| circRNA sequester (endogenous circRNA) | circRNA-circRNA (antisense) |

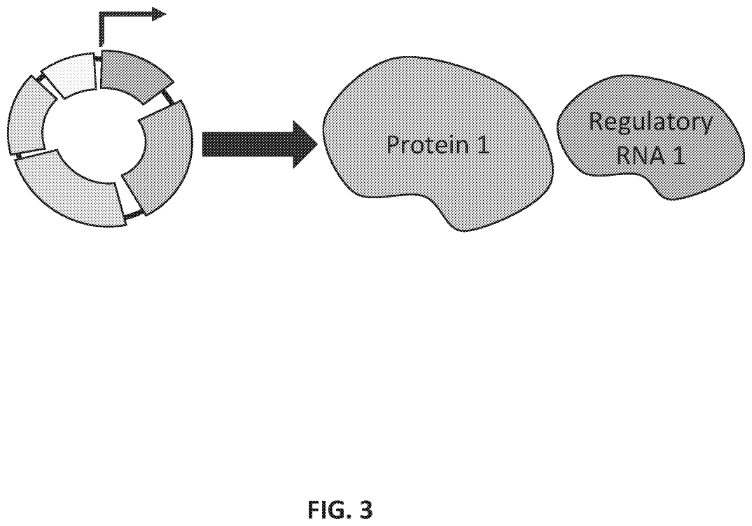

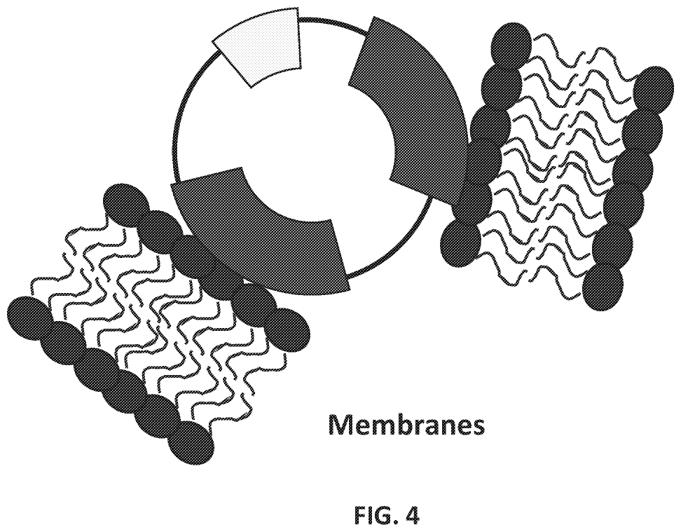

In some embodiments, any of the methods of using circRNA described herein can be in combination with a translating element. CircRNA described herein that contain a translating element can translate RNA into proteins. FIG. 3 illustrates a schematic of protein expression facilitated by a circRNA containing a sequence-specific RNA-binding motif, sequence-specific DNA-binding motif, protein-specific binding motif (Protein 1), and regulatory RNA motif (RNA 1). The regulatory RNA motif can initiate RNA transcription and protein expression.

### Untranslated Regions

In some embodiments, a circRNA as disclosed herein can comprise an encryptogen. In some embodiments, the encryptogen comprises untranslated regions (UTRs). UTRs of a gene can be transcribed but not translated. In some embodiments, a UTR can be included upstream of the translation initiation sequence of an expression sequence described herein. In some embodiments, a UTR can be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

In some embodiments, the encryptogen enhances stability. In some embodiments, the regulatory features of a UTR can be included in the encryptogen to enhance the stability of the circular polyribonucleotide.

In some embodiments, the circular polyribonucleotide comprises a UTR with one or more stretches of adenosines and uridines embedded within. AU-rich signatures can increase turnover rates of the expression product.

Introduction, removal, or modification of UTR AU-rich elements (AREs) can be useful to modulate the stability or immunogenicity of the circular polyribonucleotide. When engineering specific circular polyribonucleotides, one or more copies of an ARE can be introduced to destabilize the circular polyribonucleotide and the copies of an ARE can decrease translation and/or decrease production of an expression product. Likewise, AREs can be identified and removed or mutated to increase the intracellular stability and thus increase translation and production of the resultant protein.

A UTR from any gene can be incorporated into the respective flanking regions of the circular polyribonucleotide. Furthermore, multiple wild-type UTRs of any known gene can be utilized. In some embodiments, artificial UTRs that are not variants of wild type genes can be used. These UTRs or portions thereof can be placed in the same orientation as in the transcript from which they were selected or can be altered in orientation or location. Hence a 5′- or 3′-UTR can be inverted, shortened, lengthened, or made chimeric with one or more other 5′- or 3′-UTRs. As used herein, the term “altered” as it relates to a UTR sequence, means that the UTR has been changed in some way in relation to a reference sequence. For example, a 3′- or 5′-UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double UTR, triple UTR, or quadruple UTR, such as a 5′- or 3′-UTR, can be used. As used herein, a “double” UTR is one in which two copies of the same UTR are encoded either in series or substantially in series. For example, a double beta-globin 3′-UTR can be used in some embodiments of the invention.

### Encryptogen

As described herein, a circular polyribonucleotide can comprise an encryptogen to reduce, evade, or avoid the innate immune response of a cell. In some embodiments, circular polyribonucleotides provided herein result in a reduced immune response from the host as compared to the response triggered by a reference compound, e.g., a linear polynucleotide corresponding to the described circular polyribonucleotide or a circular polyribonucleotide lacking an encryptogen. In some embodiments, the circular polyribonucleotide has less immunogenicity than a counterpart lacking an encryptogen.

In some embodiments, the circular polyribonucleotide is non-immunogenic in a mammal, e.g., a human. In some embodiments, the circular polyribonucleotide is capable of replicating in a mammalian cell, e.g., a human cell.

In some embodiments, the circular polyribonucleotide includes sequences or expression products.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time there between.

In some embodiments, the circular polyribonucleotide is at least about 20 base pairs, at least about 30 base pairs, at least about 40 base pairs, at least about 50 base pairs, at least about 75 base pairs, at least about 100 base pairs, at least about 200 base pairs, at least about 300 base pairs, at least about 400 base pairs, at least about 500 base pairs, or at least about 1,000 base pairs. In some embodiments, the circular polyribonucleotide can be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA can be produced from DNA and their 5′ and 3′ free ends annealed to produce a “string” of RNA, which ultimately can be circularized when only one 5′ and one 3′ free end remains. In some embodiments, the maximum size of a circular polyribonucleotide can be limited by the ability of packaging and delivering the RNA to a target. In some embodiments, the size of a circular polyribonucleotide is a length sufficient to encode useful polypeptides, and thus, lengths of less than about 20,000 base pairs, less than about 15,000 base pairs, less than about 10,000 base pairs, less than about 7,500 base pairs, or less than about 5,000 base pairs, less than about 4,000 base pairs, less than about 3,000 base pairs, less than about 2,000 base pairs, less than about 1,000 base pairs, less than about 500 base pairs, less than about 400 base pairs, less than about 300 base pairs, less than about 200 base pairs, less than about 100 base pairs can be useful.

### Cleavage Sequences

In some embodiments, the circular polyribonucleotide includes at least one cleavage sequence. In some embodiments, the cleavage sequence is adjacent to an expression sequence. In some embodiments, the circular polyribonucleotide includes a cleavage sequence, such as in an immolating circRNA or cleavable circRNA or self-cleaving circRNA. In some embodiments, the circular polyribonucleotide comprises two or more cleavage sequences, leading to separation of the circular polyribonucleotide into multiple products, e.g., miRNAs, linear RNAs, smaller circular polyribonucleotide, etc.

In some embodiments, the cleavage sequence includes a ribozyme RNA sequence. A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

### Immolating Sequence

In some embodiments, circRNA described herein comprises immolating circRNA or cleavable circRNA or self-cleaving circRNA. CircRNA can deliver cellular components including, for example, RNA, lncRNA, lincRNA, miRNA, tRNA, rRNA, snoRNA, ncRNA, siRNA, or shRNA. In some embodiments, circRNA includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes. Non-limiting examples of circRNA immolating applications are listed in TABLE 4.

TABLE 3

| Process | MOA (example) |
|---|---|
| miRNA delivery | microRNAs in a circular form with self cleavage |
| | element (e.g., hammerhead), cleavage recruitment |
| | (e.g., ADAR), or degradable linker (e.g., glycerol) |
| siRNA delivery | siRNAs in circular form with self cleavage |
| | element (e.g., hammerhead), cleavage recruitment |
| | (e.g., ADAR), or degradable linker (e.g., glycerol) |

### Expression Sequences

Peptides or Polypeptides

In some embodiments, the circular polyribonucleotide comprises a sequence that encodes a peptide or polypeptide.

The polypeptide can be linear or branched. The polypeptide can have a length from about 5 to about 4000 amino acids, about 15 to about 3500 amino acids, about 20 to about 3000 amino acids, about 25 to about 2500 amino acids, about 50 to about 2000 amino acids, or any range there between. In some embodiments, the polypeptide has a length of less than about 4000 amino acids, less than about 3500 amino acids, less than about 3000 amino acids, less than about 2500 amino acids, or less than about 2000 amino acids, less than about 1500 amino acids, less than about 1000 amino acids, less than about 900 amino acids, less than about 800 amino acids, less than about 700 amino acids, less than about 600 amino acids, less than about 500 amino acids, less than about 400 amino acids, less than about 300 amino acids, or less can be useful.

In some embodiments, the circular polyribonucleotide comprises one or more RNA sequences, each of which can encode a polypeptide. The polypeptide can be produced in substantial amounts. As such, the polypeptide can be any proteinaceous molecule that can be produced. A polypeptide can be a polypeptide that can be secreted from a cell, or localized to the cytoplasm, nucleus or membrane compartment of a cell.

In some embodiments, the circular polyribonucleotide includes a sequence encoding a protein e.g., a therapeutic protein. Some examples of therapeutic proteins can include, but are not limited to, an protein replacement, protein supplementation, vaccination, antigens (e.g., tumor antigens, viral, and bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

Regulatory Sequences

In some embodiments, the regulatory sequence is a promoter. In some embodiments, the circular polyribonucleotide includes at least one promoter adjacent to at least one expression sequence. In some embodiments, the circular polyribonucleotide includes a promoter adjacent each expression sequence. In some embodiments, the promoter is present on one or both sides of each expression sequence, leading to separation of the expression products, e.g., peptide(s) and or polypeptide(s).

The circular polyribonucleotide can modulate expression of RNA encoded by a gene. Because multiple genes can share some degree of sequence homology with each other, the circular polyribonucleotide can be designed to target a class of genes with sufficient sequence homology. In some embodiments, the circular polyribonucleotide can contain a sequence that has complementarity to sequences that are shared amongst different gene targets or are unique for a specific gene target. In some embodiments, the circular polyribonucleotide can be designed to target conserved regions of an RNA sequence having homology between several genes thereby targeting several genes in a gene family. In some embodiments, the circular polyribonucleotide can be designed to target a sequence that is unique to a specific RNA sequence of a single gene.

In some embodiments, the expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, the expression sequence comprises one or more of the features described herein, e.g., a sequence encoding one or more peptides or proteins, one or more regulatory nucleic acids, one or more non-coding RNA, and other expression sequences.

Internal Ribosome Entry Site (IRES)

In some embodiments, the circular polyribonucleotides described herein comprise an internal ribosome entry site (IRES) element. A suitable IRES element can contain an RNA sequence capable of engaging a eukaryotic ribosome. In some embodiments, the IRES element is at least about 50 base pairs, at least about 100 base pairs, at least about 200 base pairs, at least about 250 base pairs, at least about 350 base pairs, or at least about 500 base pairs. In some embodiments, the IRES element is derived from the DNA of an organism including, but not limited to, a virus, a mammal, and a Drosophila. Viral DNA can be derived from, for example, picornavirus cDNA, encephalomyocarditis virus (EMCV) cDNA, and poliovirus cDNA. In some embodiments, Drosophila DNA from which an IRES element is derived can include, for example, an Antennapedia gene from Drosophila melanogaster.

In some embodiments, circular polyribonucleotides described herein include at least one IRES flanking at least one (e.g., 2, 3, 4, 5 or more) expression sequence. In some embodiments, the IRES can flank both sides of at least one (e.g., 2, 3, 4, 5 or more) expression sequence. In some embodiments, circular polyribonucleotides can include one or more IRES sequences on one or both sides of each expression sequence, leading to separation of the resulting peptide(s) and or polypeptide(s).

Translation Initiation Sequence

In some embodiments, the circular polyribonucleotide encodes a polypeptide and can comprise a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence.

Natural 5′-UTRs can bear features that play a role in translation initiation. Natural 5′-UTRs can harbor signatures like Kozak sequences, which can be involved in the process by which the ribosome initiates translation of many genes. Kozak sequences have the consensus CCR(A/G)CCAUGG, where R is a purine (adenine or guanine) three bases upstream of the start codon (AUG), which is followed by another “G”. 5′-UTR can also form secondary structures that are involved in elongation factor binding.

The circular polyribonucleotide can include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60, or more than 60 start codons. Translation can initiate on the first start codon or initiate downstream of the first start codon.

In some embodiments, the circular polyribonucleotide can initiate at a codon that is not the first start codon, e.g., AUG. Translation of the circular polyribonucleotide can initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the circular polyribonucleotide can begin at alternative translation initiation sequence, such as ACG. As another non-limiting example, the circular polyribonucleotide translation can begin at alternative translation initiation sequence, CTG/CUG. As yet another non-limiting example, the circular polyribonucleotide translation can begin at alternative translation initiation sequence, GTG/GUG. As yet another non-limiting example, the circular polyribonucleotide can begin translation at a repeat-associated non-AUG (RAN) sequence, such as an alternative translation initiation sequence that includes short stretches of repetitive RNA, e.g., CGG, GGGGCC, CAG, CTG.

Nucleotides flanking a codon that initiates translation can affect the translation efficiency, the length and/or the structure of the circular polyribonucleotide. Masking any of the nucleotides flanking a codon that initiates translation can be used to alter the position of translation initiation, translation efficiency, length, and/or structure of the circular polyribonucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon-junction complexes (EJCs). In some embodiments, a masking agent can be used to mask a start codon of the circular polyribonucleotide in order to increase the likelihood that translation will initiate at an alternative start codon.

In some embodiments, translation is initiated under selective conditions, such as, but not limited to, viral induced selection in the presence of GRSF-1 and the circular polyribonucleotide includes GRSF-1 binding sites.

In some embodiments, translation is initiated by eukaryotic initiation factor 4A (eIF4A) treatment with Rocaglates. Translation can be repressed by blocking 43 S scanning, leading to premature, upstream translation initiation and reduced protein expression from transcripts bearing the RocA-eIF4A target sequence.

Termination Sequence

In some embodiments, the circular polyribonucleotide includes one or more expression sequences and each expression sequence can have a termination sequence. In some embodiments, the circular polyribonucleotide includes one or more expression sequences and the expression sequences lack a termination sequence, such that the circular polyribonucleotide is continuously translated. Exclusion of a termination sequence can result in rolling circle translation or continuous production of expression product, e.g., peptides or polypeptides, due to lack of ribosome stalling or fall-off. In such an embodiment, rolling circle translation produces a continuous expression product through each expression sequence.

In some embodiments, the circular polyribonucleotide includes a stagger sequence. To avoid production of a continuous expression product, e.g., peptide or polypeptide, while maintaining rolling circle translation, a stagger sequence can be included to induce ribosomal pausing during translation. The stagger sequence can include a 2A-like or CHYSEL (cis-acting hydrolase element) sequence. In some embodiments, the stagger element encodes a sequence with a C-terminal consensus sequence that is X1X2X3EX5NPGP, where X1 is absent or G or H, X2 is absent or D or G, X3 is D or V or I or S or M, and X5 is any amino acid. In some embodiments, this sequence comprises a non-conserved sequence of amino-acids with a strong alpha-helical propensity followed by the consensus sequence −D(V/I)ExNPG P, where x=any amino acid. Some nonlimiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the circular polyribonucleotide includes a termination sequence at the end of one or more expression sequences. In some embodiments, one or more expression sequences lacks a termination sequence. Generally, termination sequences include an in-frame nucleotide triplet that signals termination of translation, e.g., UAA, UGA, UAG. In some embodiments, one or more termination sequences in the circular polyribonucleotide are frame-shifted termination sequences, such as but not limited to, off-frame or −1 and +1 shifted reading frames (e.g., hidden stop) that can terminate translation. Frame-shifted termination sequences include nucleotide triples, TAA, TAG, and TGA that appear in the second and third reading frames of an expression sequence. Frame-shifted termination sequences can be important in preventing misreads of mRNA, which is often detrimental to the cell.

In some embodiments, a stagger sequence described herein can terminate translation and/or cleave an expression product between G and P of the consensus sequence described herein. As one non-limiting example, the circular polyribonucleotide includes at least one stagger sequence to terminate translation and/or cleave the expression product. In some embodiments, the circular polyribonucleotide includes a stagger sequence adjacent to at least one expression sequence. In some embodiments, the circular polyribonucleotide includes a stagger sequence after each expression sequence. In some embodiments, the circular polyribonucleotide includes a stagger sequence is present on one or both sides of each expression sequence, leading to translation of individual peptide(s) and or polypeptide(s) from each expression sequence.

PolyA Sequence

In some embodiments, the circular polyribonucleotide includes a poly-A sequence. In some embodiments, the length of a poly-A sequence is greater than 10 nucleotides in length. In some embodiments, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A sequence is designed relative to the length of the overall circular polyribonucleotide. The design can be based on the length of the coding region, the length of a particular feature or region (such as the first or flanking regions), or based on the length of the ultimate product expressed from the circular polyribonucleotide. In this context, the poly-A sequence can be 10, 20, 30, 40, 50, 60, 70, 80, 90, or 100% greater in length than the circular polyribonucleotide or a feature thereof. The poly-A sequence can also be designed as a fraction of the circular polyribonucleotide. In this context, the poly-A sequence can be 10%, 20%, 30%, 40%, 50%, 60%, 70%, 80%, 90%, or more of the total length of the construct or the total length of the construct minus the poly-A sequence. Further, engineered binding sites and conjugation of circular polyribonucleotide for Poly-A binding protein can enhance expression.

In some embodiments, the circular polyribonucleotide is designed to include a polyA-G quartet. The G-quartet is a cyclic hydrogen bonded array of four guanine nucleotides that can be formed by G-rich sequences in both DNA and RNA. In some embodiments, the G-quartet can be incorporated at the end of the poly-A sequence. The resultant circular polyribonucleotide construct can be assayed for stability, protein production, and/or other parameters including half-life at various time points. In some embodiments, the polyA-G quartet can result in protein production equivalent to at least 75% of that seen using a poly-A sequence of 120 nucleotides alone.

### Riboswitches

In some embodiments, the circular polyribonucleotide comprises one or more riboswitches.

A riboswitch can be a part of the circular polyribonucleotide that can directly bind a small target molecule, and whose binding of the target affects RNA translation and the expression product stability and activity. Thus, the circular polyribonucleotide that includes a riboswitch can regulate the activity of the circular polyribonucleotide depending on the presence or absence of the target molecule. In some embodiments, a riboswitch has a region of aptamer-like affinity for a separate molecule. Any aptamer included within a non-coding nucleic acid can be used for sequestration of molecules from bulk volumes. In some embodiments, “(ribo)switch” activity can be used for downstream reporting of the event.

In some embodiments, the riboswitch modulates gene expression by transcriptional termination, inhibition of translation initiation, mRNA self-cleavage, and in eukaryotes, alteration of splicing pathways. The riboswitch can control gene expression through the binding or removal of a trigger molecule. Thus, subjecting a circular polyribonucleotide that includes the riboswitch to conditions that activate, deactivate, or block the riboswitch can alter gene expression. For example, gene expression can be altered as a result of termination of transcription or blocking of ribosome binding to the RNA. Binding of a trigger molecule, or an analog thereof, can reduce/prevent expression or promote/increase expression of the RNA molecule depending on the nature of the riboswitch.

In some embodiments, the riboswitch is a Cobalamin riboswitch (also B12-element), which binds adenosylcobalamin (the coenzyme form of vitamin B12) to regulate the biosynthesis and transport of cobalamin and similar metabolites.

In some embodiments, the riboswitch is a cyclic di-GMP riboswitch, which binds cyclic di-GMP to regulate a variety of genes. There are two non-structurally related classes of cyclic di-GMP riboswitch: cyclic di-GMP-I and cyclic di-GMP-II.

In some embodiments, the riboswitch is a FMN riboswitch (also RFN-element) which binds flavin mononucleotide (FMN) to regulate riboflavin biosynthesis and transport.

In some embodiments, the riboswitch is a glmS riboswitch, which cleaves itself when there is a sufficient concentration of glucosamine-6-phosphate.

In some embodiments, the riboswitch is a glutamine riboswitch, which binds glutamine to regulate genes involved in glutamine and nitrogen metabolism. Glutamine riboswitches can also bind short peptides of unknown function. Such riboswitches fall into two structurally related classes: the glnA RNA motif and Downstream-peptide motif.

In some embodiments, the riboswitch is a glycine riboswitch, which binds glycine to regulate glycine metabolism genes. It comprises two adjacent aptamer domains in the same mRNA, and is the only known natural RNA that exhibits cooperative binding.

In some embodiments, the riboswitch is a lysine riboswitch (also L-box), which binds lysine to regulate lysine biosynthesis, catabolism, and transport.

In some embodiments, the riboswitch is a preQ1 riboswitch, which binds pre-queuosine to regulate genes involved in the synthesis or transport of this precursor to queuosine. Two distinct classes of preQ1 riboswitches are preQ1-I riboswitches and preQ1-II riboswitches. The binding domain of preQ1-I riboswitches is unusually small among naturally occurring riboswitches. PreQ1-II riboswitches, which are only found in certain species in the genera Streptococcus and Lactococcus, have a completely different structure and are larger than preQ1-I riboswitches.

In some embodiments, the riboswitch is a purine riboswitch, which binds purines to regulate purine metabolism and transport. Different forms of purine riboswitches bind guanine or adenine. The specificity for either guanine or adenine depends upon Watson-Crick interactions with a single pyrimidine in the riboswitch at position Y74. In the guanine riboswitch, the single pyrimidine is cytosine (i.e., C74). In the adenine riboswitch, the single pyrimidine is uracil (i.e., U74). Homologous types of purine riboswitches can bind deoxyguanosine, but have more significant differences than a single nucleotide mutation.

In some embodiments, the riboswitch is an S-adenosylhomocysteine (SAH) riboswitch, which binds SAH to regulate genes involved in recycling SAH produced from S-adenosylmethionine (SAM) in methylation reactions.

In some embodiments, the riboswitch is an S-adenosyl methionine (SAM) riboswitch, which binds SAM to regulate methionine and SAM biosynthesis and transport. There are three distinct SAM riboswitches: SAM-I (originally called S-box), SAM-II, and the SMK box. SAM-I is widespread in bacteria. SAM-II is found only in α-, β-, and a few γ-proteobacteria. The SMK box riboswitch is found in Lactobacillales. These three varieties of riboswitch have no obvious sequence or structural similarities. A fourth variety, SAM-IV, appears to have a similar ligand-binding core to that of SAM-I, but in the context of a distinct scaffold.

In some embodiments, the riboswitch is a SAM-SAH riboswitch, which binds both SAM and SAH with similar affinities.

In some embodiments, the riboswitch is a tetrahydrofolate riboswitch, which binds tetrahydrofolate to regulate synthesis and transport genes.

In some embodiments, the riboswitch is a theophylline-binding riboswitch or a thymine pyrophosphate-binding riboswitch.

In some embodiments, the riboswitch is a glmS catalytic riboswitch from Thermoanaerobacter tengcongensis, which senses glucosamine-6 phosphate.

In some embodiments, the riboswitch is a thiamine pyrophosphate (TPP) riboswitch (also Thi-box), which binds TPP to regulate thiamine biosynthesis and transport, as well as transport of similar metabolites. The TPP riboswitch is found in eukaryotes.

In some embodiments, the riboswitch is a Moco riboswitch, which binds molybdenum cofactor, to regulate genes involved in biosynthesis and transport of this coenzyme, as well as enzymes that use molybdenum or derivatives thereof as a cofactor.

In some embodiments, the riboswitch is an adenine-sensing add-A riboswitch, found in the 5′-UTR of the adenine deaminase (add) encoding gene of Vibrio vulnificus.

### Aptazyme

In some embodiments, the circular polyribonucleotide comprises an aptazyme. Aptazyme is a switch for conditional expression in which an aptamer region is used as an allosteric control element and coupled to a region of catalytic RNA (a “ribozyme” as described below). In some embodiments, the aptazyme is active in cell type-specific translation. In some embodiments, the aptazyme is active under cell state-specific translation, e.g., virally infected cells or in the presence of viral nucleic acids or viral proteins.

A ribozyme is a RNA molecule that catalyzes a chemical reaction. Many natural ribozymes can catalyze the hydrolysis of phosphodiester bonds of the ribozyme itself or the hydrolysis of phosphodiester bonds in other RNA. Natural ribozymes can also catalyze the aminotransferase activity of the ribosome. Catalytic RNA can be “evolved” by in vitro methods. Ribozymes and reaction products of ribozymes can regulate gene expression. In some embodiments, a catalytic RNA or ribozyme can be placed within a larger, non-coding RNA such that the ribozyme is present at many copies within the cell for chemical transformation of a molecule from a bulk volume. In some embodiments, aptamers and ribozymes can both be encoded in the same non-coding RNA.

Non-limiting examples of ribozymes include hammerhead ribozyme, VL ribozyme, leadzyme, and hairpin ribozyme.

In some embodiments, the aptazyme is a ribozyme that can cleave RNA sequences and can be regulated as a result of binding a ligand or modulator. The ribozyme can be a self-cleaving ribozyme. As such, these ribozymes can combine the properties of ribozymes and aptamers.

In some embodiments, the aptazyme is included in an untranslated region of circular polyribonucleotides described herein. An aptazyme in the absence of ligand/modulator is inactive, which can allow expression of the transgene. Expression can be turned off or down-regulated by addition of the ligand. Aptazymes that are downregulated in response to the presence of a particular modulator can be used in control systems where upregulation of gene expression in response to modulator is desired.

Aptazymes can also be used to develop of systems for self-regulation of circular polyribonucleotide expression. For example, the protein product of circular polyribonucleotides described herein that is the rate determining enzyme in the synthesis of a particular small molecule can be modified to include an aptazyme that is selected to have increased catalytic activity in the presence of the small molecule to provide an autoregulatory feedback loop for synthesis of the molecule. Alternatively, the aptazyme activity can be selected sense accumulation of the protein product from the circular polyribonucleotide, or any other cellular macromolecule.

In some embodiments, the circular polyribonucleotide can include an aptamer sequence. Non-limiting examples of aptamers include RNA aptamers that bind lysozyme, Toggle-25t (an RNA aptamer containing 2′-fluoropyrimidine nucleotides that binds thrombin with high specificity and affinity), RNA-Tat that binds human immunodeficiency virus trans-acting responsive element (HIV TAR), RNA aptamers that bind hemin, RNA aptamers that bind interferon γ, RNA aptamer binding vascular endothelial growth factor (VEGF), RNA aptamers that bind prostate specific antigen (PSA), RNA aptamers that bind dopamine, and RNA aptamers that bind heat shock factor 1 (HSF1).

In some embodiments, circRNA described herein can be used for transcription and replication of RNA. For example, circRNA can be used to encode non-coding RNA, lncRNA, miRNA, tRNA, rRNA, snoRNA, ncRNA, siRNA, or shRNA. In some embodiments, circRNA can include anti-sense miRNA and a transcriptional element. After transcription, such circRNA can produce functional, linear miRNAs. Non-limiting examples of circRNA expression and modulation applications are listed in TABLE 5.

TABLE 4

| Process | MOA (example) |
|---|---|
| Combinational therapy of | Inhibition of one protein and |
| inhibition & translation | supplementation of another (or same) |

### Replication Element

The circular polyribonucleotide can encode a sequence and/or motif useful for replication. Replication of a circular polyribonucleotide can occur by generating a complement circular polyribonucleotide. In some embodiments, the circular polyribonucleotide includes a motif to initiate transcription, where transcription is driven by either endogenous cellular machinery (DNA-dependent RNA polymerase) or an RNA-depended RNA polymerase encoded by the circular polyribonucleotide. The product of rolling-circle transcriptional event can be cut by a ribozyme to generate either complementary or propagated circular polyribonucleotide at unit length. The ribozymes can be encoded by the circular polyribonucleotide, its complement, or by an RNA sequence in trans. In some embodiments, the encoded ribozymes can include a sequence or motif that regulates (inhibits or promotes) activity of the ribozyme to control circRNA propagation. In some embodiments, unit-length sequences can be ligated into a circular form by a cellular RNA ligase. In some embodiments, the circular polyribonucleotide includes a replication element that aids in self-amplification. Examples of such replication elements include HDV replication domains and replication competent circular RNA sense and/or antisense ribozymes, such as antigenomic

(SEQ ID NO: 1)
5′-CGGGUCGGCAUGGCAUCUCCACCUCCUCGCGGUCCGACCUGGGCAUCC
GAAGGAGGACGCACGUCCACUCGGAUGGCUAAGGGAGAGCCA-3′
or genomic
(SEQ ID NO: 2)
5′-UGGCCGGCAUGGUCCCAGCCUCCUCGCUGGCGCCGGCUGGGCAACAUU
CCGAGGGGACCGUCCCCUCGGUAAUGGCGAAUGGGACCCA-3′.

In some embodiments, the circular polyribonucleotide includes at least one cleavage sequence as described herein to aid in replication. A cleavage sequence within the circular polyribonucleotide can cleave long transcripts replicated from the circular polyribonucleotide to a specific length that can subsequently circularize to form a complement to the circular polyribonucleotide.

In another embodiment, the circular polyribonucleotide includes at least one ribozyme sequence to cleave long transcripts replicated from the circular polyribonucleotide to a specific length, where another encoded ribozyme cuts the transcripts at the ribozyme sequence. Circularization forms a complement to the circular polyribonucleotide.

In some embodiments, the circular polyribonucleotide is substantially resistant to degradation, e.g., by exonucleases.

In some embodiments, the circular polyribonucleotide replicates within a cell. In some embodiments, the circular polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage there between. In some embodiments, the circular polyribonucleotide is replicates within a cell and is passed to daughter cells. In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

In some embodiments, the circular polyribonucleotide replicates within the host cell. In some embodiments, the circular polyribonucleotide is capable of replicating in a mammalian cell, e.g., human cell.

While in some embodiments the circular polyribonucleotide replicates in the host cell, the circular polyribonucleotide does not integrate into the genome of the host, e.g., with the host's chromosomes. In some embodiments, the circular polyribonucleotide has a negligible recombination frequency, e.g., with the host's chromosomes. In some embodiments, the circular polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### Other Sequences

In some embodiments, the circular polyribonucleotide further includes another nucleic acid sequence. In some embodiments, the circular polyribonucleotide can include DNA, RNA, or artificial nucleic acid sequences. The other sequences can include, but are not limited to, genomic DNA, cDNA, or sequences that encode tRNA, mRNA, rRNA, miRNA, gRNA, siRNA, or other RNAi molecules. In some embodiments, the circular polyribonucleotide includes a sequence encoding an siRNA to target a different locus or loci of the same gene expression product as the circular polyribonucleotide. In some embodiments, the circular polyribonucleotide includes a sequence encoding an siRNA to target a different gene expression product as the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination sequence. In some embodiments, the circular polyribonucleotide lacks an internal ribosomal entry site. In some embodiments, the circular polyribonucleotide lacks degradation susceptibility by exonucleases. In some embodiments, the circular polyribonucleotide lacks binding to cap-binding proteins. In some embodiments, the circular polyribonucleotide lacks a 5′ cap.

In some embodiments, the circular polyribonucleotide comprises one or more of the following sequences: a sequence that encodes one or more miRNA, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNA, shRNA), and a sequence that encodes a therapeutic mRNA or protein.

The other sequence can have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range there between.

As a result of its circularization, the circular polyribonucleotide can include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide is less susceptible to dephosphorylation when the circular polyribonucleotide is incubated with phosphatase, such as calf intestine phosphatase.

### Nucleotide Spacer Sequences

In some embodiments, the circular polyribonucleotide comprises a spacer sequence.

The spacer can be a nucleic acid molecule having low GC content, for example less than 65%, 60%, 55%, 50%, 55%, 50%, 45%, 40%, 39%, 38%, 37%, 36%, 35%, 34%, 33%, 32%, 31%, 30%, 29%, 28%, 27%, 26%, 25%, 24%, 23%, 22%, 20%, 19%, 18%, 17%, 16%, 15%, 14%, 13%, 12%, 11%, 10%, 9%, 8%, 7%, 6%, 5%, 4%, 3%, 2% or 1%, across the full length of the spacer, or across at least 50%, 60%, 70%, 80%, 90%, 91%, 92%, 93%, 94%, 95%, 96%, 97%, 98% or 99% contiguous nucleic acid residues of the spacer. In some embodiments, the spacer is substantially free of a secondary structure, such as less than 40 kcal/mol, less than −39, −38, −37, −36, −35, −34, −33, −32, −31, −30, −29, −28, −27, −26, −25, −24, −23, −22, −20, −19, −18, −17, −16, −15, −14, −13, −12, −11, −10, −9, −8, −7, −6, −5, −4, −3, −2 or −1 kcal/mol. The spacer can include a nucleic acid, such as DNA or RNA.

The spacer sequence can encode an RNA sequence, and preferably a protein or peptide sequence, including a secretion signal peptide.

The spacer sequence can be non-coding. Where the spacer is a non-coding sequence, a start codon can be provided in the coding sequence of an adjacent sequence. In some embodiments, it is envisaged that the first nucleic acid residue of the coding sequence can be the A residue of a start codon, such as AUG. Where the spacer encodes an RNA or protein or peptide sequence, a start codon can be provided in the spacer sequence.

In some embodiments, the spacer is operably linked to another sequence described herein.

### Non-Nucleic Acid Linkers

The circular polyribonucleotide described herein can also comprise a non-nucleic acid linker. In some embodiments, the circular polyribonucleotide described herein has a non-nucleic acid linker between one or more of the sequences or elements described herein. In some embodiments, one or more sequences or elements described herein are linked with the linker. The non-nucleic acid linker can be a chemical bond, e.g., one or more covalent bonds or non-covalent bonds. In some embodiments, the non-nucleic acid linker is a peptide or protein linker. Such a linker can be between 2-30 amino acids, or longer. The linker includes flexible, rigid or cleavable linkers described herein.

The most commonly used flexible linkers have sequences consisting primarily of stretches of Gly and Ser residues (“GS” linker). Flexible linkers can be useful for joining domains that require a certain degree of movement or interaction and can include small, non-polar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers can also be useful when a spatial separation of the domains is critical to preserve the stability or bioactivity of one or more components in the fusion. Rigid linkers can have an alpha helix-structure or Pro-rich sequence, (XP)ₙ, with X designating any amino acid, preferably Ala, Lys, or Glu.

Cleavable linkers can release free functional domains in vivo. In some embodiments, linkers can be cleaved under specific conditions, such as the presence of reducing reagents or proteases. In vivo cleavable linkers can utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. In vivo cleavage of linkers in fusions can also be carried out by proteases that are expressed in vivo under pathological conditions (e.g., cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (—CH₂-ipids, such as a poly (—CHe g polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide can be linked using charge-based chemistry, such that a positively charged moiety of the polypeptide is linked to a negative charge of another polypeptide or nucleic acid.

### Circularization

In some embodiments, a linear circular polyribonucleotide can be cyclized or concatemerized. In some embodiments, the linear circular polyribonucleotide can be cyclized in vitro prior to formulation and/or delivery. In some embodiments, linear circular polyribonucleotides can be cyclized within a cell.

### Extracellular Circularization

In some embodiments, a linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, can form a new covalent linkage between the 5′-end and the 3′-end of the molecule. The 5′-end can contain an NETS-ester reactive group and the 3′-end can contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′- or 3′-amide bond.

In some embodiments, a DNA or RNA ligase can be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphodiester linkage. In an example reaction, a linear circular polyribonucleotide is incubated at 37° C. for 1 hour with 1-10 units of T4 RNA ligase according to the manufacturer's protocol. The ligation reaction can occur in the presence of a linear nucleic acid capable of base-pairing with both the 5′- and 3′-region in juxtaposition to assist the enzymatic ligation reaction.

In some embodiments, a DNA or RNA ligase can be used in the synthesis of the circular polynucleotides. As a non-limiting example, the ligase can be a circ ligase or circular ligase.

In some embodiments, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme can be derived from the Group I Intron, Hepatitis Delta Virus, Hairpin ribozyme or can be selected by SELEX (systematic evolution of ligands by exponential enrichment). The ribozyme ligase reaction can take 1 to 24 hours at temperatures between 0 and 37° C.

In some embodiments, a linear circular polyribonucleotide can be cyclized or concatermerized by using at least one non-nucleic acid moiety. In one aspect, the at least one non-nucleic acid moiety can react with regions or features near the 5′-terminus and/or near the 3′-terminus of the linear circular polyribonucleotide in order to cyclize or concatermerize the linear circular polyribonucleotide. In another aspect, the at least one non-nucleic acid moiety can be located in or linked to or near the 5′-terminus and/or the 3′-terminus of the linear circular polyribonucleotide. The non-nucleic acid moieties contemplated can be homologous or heterologous. As a non-limiting example, the non-nucleic acid moiety can be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety can be an oligonucleotide or a peptide moiety, such as an aptamer or a non-nucleic acid linker as described herein.

In some embodiments, a linear circular polyribonucleotide can be cyclized or concatermerized due to a non-nucleic acid moiety that causes an attraction between atoms, molecular surfaces at, near or linked to the 5′- and 3′-ends of the linear circular polyribonucleotide. As a non-limiting example, one or more linear circular polyribonucleotides can be cyclized or concantermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide can comprise a ribozyme RNA sequence near the 5′-terminus and near the 3′-terminus. The ribozyme RNA sequence can covalently link to a peptide when the sequence is exposed to the remainder of the ribozyme. In one aspect, the peptides covalently linked to the ribozyme RNA sequence near the 5′-terminus and the 3′-terminus can associate with each other causing a linear circular polyribonucleotide to cyclize or concatemerize. In another aspect, the peptides covalently linked to the ribozyme RNA near the 5′-terminus and the 3′-terminus can cause the linear primary construct or linear mRNA to cyclize or concatemerize after being subjected to ligation using various methods known in the art such as, but not limited to, protein ligation.

In some embodiments, the linear circular polyribonucleotide can include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate can occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

### Splicing Element

In some embodiment, the circular polyribonucleotide includes at least one splicing element. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, the circular polyribonucleotide includes an internal splicing element that when replicated the spliced ends are joined together. Some examples can include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence can include repeated sequences from the Alu family of introns. In some embodiments, a splicing-related ribosome binding protein can regulate circular polyribonucleotide biogenesis, e.g., the Muscleblind and Quaking (QKI) splicing factors.

In some embodiments, the circular polyribonucleotide can include canonical splice sites that flank head-to-tail junctions of the circular polyribonucleotide.

In some embodiments, the circular polyribonucleotide can include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′—OH group onto the 2′, 3′-cyclic phosphate of the same molecule forming a 3′,5′-phosphodiester bridge.

In some embodiments, the circular polyribonucleotide can include a multimeric repeating RNA sequence that harbors a HPR element. The HPR comprises a 2′,3′-cyclic phosphate and a 5′-OH termini. The HPR element self-processes the 5′- and 3′-ends of the linear circular polyribonucleotide, thereby ligating the ends together.

In some embodiments, the circular polyribonucleotide can include a sequence that mediates self-ligation. In some embodiments, the circular polyribonucleotide can include a HDV sequence (e.g., HDV replication domain conserved sequence, GGCUCAUCUCGACAAGAGGCGGCAGUCCUCAGUACUCUUACUCUUUUCUGUAAA GAGGAGACUGCUGGACUCGCCGCCCAAGUUCGAGCAUGAGCC (SEQ ID NO: 3) (Beeharry et al 2004) or GGCUAGAGGCGGCAGUCCUCAGUACUCUUACUCUUUUCUGUAAAGAGGAGACUG CUGGACUCGCCGCCCGAGCC (SEQ ID NO: 4)) to self-ligate. In some embodiments, the circular polyribonucleotide can include loop E sequence (e.g., in PSTVd) to self-ligate. In another embodiment, the circular polyribonucleotide can include a self-circularizing intron, e.g., a 5′ and 3′-slice junction, or a self-circularizing catalytic intron such as a Group I, Group II or Group III Introns. Non-limiting examples of group I intron self-splicing sequences can include self-splicing permuted intron-exon sequences derived from T4 bacteriophage gene td, and the intervening sequence (IVS) rRNA of Tetrahymena.

### Other Circularization Methods

In some embodiments, linear circular polyribonucleotides can include complementary sequences, including either repetitive or nonrepetitive nucleic acid sequences within individual introns or across flanking introns. Repetitive nucleic acid sequences are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′- and 3′-ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

### Modifications

In some aspects, the invention described herein comprises compositions and methods of using and making modified circular polyribonucleotides, and delivery of modified circular polyribonucleotides. The term “modified nucleotide” can refer to any nucleotide analog or derivative that has one or more chemical modifications to the chemical composition of an unmodified natural ribonucleotide, such as a natural unmodified nucleotide adenosine (A), uridine (U), guaninie (G), cytidine (C) as shown by the chemical formulae in TABLE 5, and monophosphate. The chemical modifications of the modified ribonucleotide can be modifications to any one or more functional groups of the ribonucleotide, such as, the sugar the nucleobase, or the internucleoside linkage (e.g. to a linking phosphate/to a phosphodiester linkage/to the phosphodiester backbone).

TABLE 5
Unmodified Natural Ribonucleosides

| Ribonucleoside | IUPAC name | Chemical Formula |
|---|---|---|
| Adenosine | (2R,3R,4S,5R)-2- (6-amino-9H-purin- 9-yl)-5- (hydroxymethyl) oxolane-3,4-diol | 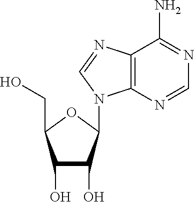 |
| | | C10H13N5O4 |
| Uridine | 1-[(3R,4S,5R)-3,4- dihydroxy-5- (hydroxymethyl) oxolan-2-yl] pyrimidine-2,4-dione | 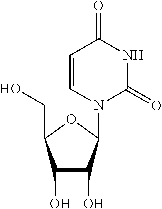 |
| | | C₉H₁₂N₂O₆ |
| Guanine | 2-amino-9H-purin- 6(1H)-one | 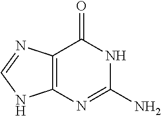 [SMILES: Nc1nc2[nH]cnc2c(=O)[nH]1] |
| | | C₅H₅N₅O |
| Cytidine | 4-amino-1- [(2R,3R,4S,5R)- 3,4-dihydroxy-5- (hydroxymethyl) oxolan-2- yl]pyrimidin- 2(1H)-one | 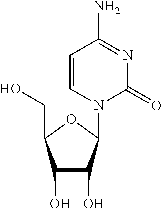 |
| | | C₉H₁₃N₃O₅ |

The circular polyribonucleotide can include one or more substitutions, insertions and/or additions, deletions, and covalent modifications with respect to reference sequences, in particular, the parent polyribonucleotide, are included within the scope of this invention. In some embodiments, the circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The circular polyribonucleotide can include any useful modification, such as to the sugar, the nucleobase, or the internucleoside linkage (e.g., to a linking phosphate/to a phosphodiester linkage/to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications can be modifications of ribonucleic acids (RNA) to deoxyribonucleic acids (DNA), threose nucleic acids (TNA), glycol nucleic acids (GNA), peptide nucleic acids (PNA), locked nucleic acids (LNA) or hybrids thereof). Additional modifications are described herein.

In some embodiments, the circular polyribonucleotide includes at least one N(6)methyladenosine (m6A) modification to increase translation efficiency.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

“Pseudouridine” refers, in another embodiment, to m¹acp³Ψ (1-methyl-3-(3-amino-3-carboxypropyl) pseudouridine. In another embodiment, the term refers to m¹Ψ (1-methylpseudouridine). In another embodiment, the term refers to Ψm (2′-O-methylpseudouridine. In another embodiment, the term refers to m5D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

In some embodiments, chemical modifications to the ribonucleotides of the circular polyribonucleotide can enhance immune evasion. Modifications include, for example, end modifications, e.g., 5′-end modifications (phosphorylation (mono-, di- and tri-), conjugation, inverted linkages, etc.), 3′-end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases can also include 5-methylcytidine and pseudouridine. In some embodiments, base modifications can modulate expression, immune response, stability, subcellular localization, to name a few functional effects, of the circular polyribonucleotide. In some embodiments, the modification includes a bi-orthogonal nucleotide, e.g., an unnatural base.

In some embodiments, sugar modifications (e.g., at the 2′ position or 4′ position) or replacement of the sugar one or more ribonucleotides of the circular polyribonucleotide can, as well as backbone modifications, include modification or replacement of the phosphodiester linkages. Non-limiting examples of circular polyribonucleotide include circular polyribonucleotide with modified backbones or non-natural internucleoside linkages, such as those modified or replaced of the phosphodiester linkages. Circular polyribonucleotides having modified backbones include, among others, those that do not have a phosphorus atom in the backbone. For the purposes of this application, and as sometimes referenced in the art, modified RNA that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide backbones can include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide can be negatively or positively charged.

The modified nucleotides, which can be incorporated into the circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In some embodiments, a modified nucleoside includes an α-thio-nucleoside (e.g., 5′-O-(l-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine). Other internucleoside linkages can include internucleoside linkages which do not contain a phosphorous atom.

In some embodiments, the circular polyribonucleotide can include one or more cytotoxic nucleosides. For example, cytotoxic nucleosides can be incorporated into circular polyribonucleotide, such as bifunctional modification. Cytotoxic nucleoside can include, but are not limited to, adenosine arabinoside, 5-azacytidine, 4′-thio-aracytidine, cyclopentenylcytosine, cladribine, clofarabine, cytarabine, cytosine arabinoside, l-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((R,S)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

The circular polyribonucleotide can be uniformly modified along the entire length of the molecule. For example, one or more or all types of nucleotide (e.g., naturally-occurring nucleotides, purine or pyrimidine, or any one or more or all of A, G, U, C, I, pU) can be uniformly modified in the circular polyribonucleotide, or in a given predetermined sequence region thereof. In some embodiments, the circular polyribonucleotide includes a pseudouridine. In some embodiments, the circular polyribonucleotide includes an inosine, which can aid in the immune system characterizing the circular polyribonucleotide as endogenous versus viral RNA. The incorporation of inosine can also mediate improved RNA stability/reduced degradation.

In some embodiments, all nucleotides in the circular polyribonucleotide (or in a given sequence region thereof) are modified. In some embodiments, the modification can include an m6A, which can augment expression; an inosine, which can attenuate an immune response; pseudouridine, which can increase RNA stability, or translational readthrough (stop codon=coding potential), an m5C, which can increase stability; and a 2,2,7-trimethylguanosine, which aids subcellular translocation (e.g., nuclear localization).

Different sugar modifications, nucleotide modifications, and/or internucleoside linkages (e.g., backbone structures) can exist at various positions in the circular polyribonucleotide. One of ordinary skill in the art will appreciate that the nucleotide analogs or other modification(s) can be located at any position(s) of the circular polyribonucleotide, such that the function of the circular polyribonucleotide is not substantially decreased. A modification can also be a non-coding region modification. The circular polyribonucleotide can include from about 1% to about 100% modified nucleotides (either in relation to overall nucleotide content, or in relation to one or more types of nucleotide, i.e., any one or more of A, G, U, or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the circular polyribonucleotide provided herein is a modified circular polyribonucleotide. For example, a completely modified circular polyribonucleotide comprises all or substantially all modified adenosine residues, all or substantially all modified uridine residues, all or substantially all modified guanine residues, all or substantially all modified cytidine residues, or any combination thereof. In some embodiments, the circular polyribonucleotide provided herein is a hybrid modified circular polyribonucleotide. A hybrid modified circular polyribonucleotide can have at least one modified nucleotide and can have a portion of contiguous unmodified nucleotides. This unmodified portion of the hybrid modified circular polyribonucleotide can have at least about 5, 10, 15, or 20 contiguous unmodified nucleotides, or any number therebetween. In some embodiments, the unmodified portion of the hybrid modified circular polyribonucleotide has at least about 30, 40, 40, 60, 70, 80, 90, 100, 110, 120, 130, 140, 150, 160, 180, 200, 220, 250, 280, 300, 320, 350, 380, 400, 420, 450, 500, 550, 600, 650, 700, 750, 800, 850, 900, or 1000 contiguous unmodified nucleotides, or any number therebetween. In some embodiments, the hybrid modified circular polyribonucleotide has 1, 2, 3, 4, 5, 6, 7, 8, 9, 10, or more unmodified portions. In some embodiments, the hybrid modified circular polyribonucleotide has at least 1, 2, 3, 4, 5, 6, 7, 8, 9, 10, 12, 15, 20, 30, 40, 50, 70, 80, 100, 120, 150, 200, 250, 300, 400, 500, 600, 700, 800, 900, 1000, or more modified nucleotides. In some embodiments, the hybrid modified circular polyribonucleotide has at least 1%, 2%, 5%, 7%, 8%, 10%, 12%, 15%, 18%, 20%, 25%, 30%, 35%, 40%, 45%, 50%, 55%, 60%, 65%, 70%, 80%, 90%, 95%, or 99% but less than 100% nucleotides that are modified. In some embodiments, the unmodified portion comprises a binding site. In some embodiments, the unmodified portion comprises a binding site configured to bind a protein, DNA, RNA, or a cell target. In some embodiments, the unmodified portion comprises an IRES.

In some embodiments, the hybrid modified circular polyribonucleotide has a lower immunogenicity than a corresponding unmodified circular polyribonucleotide. In some embodiments, the hybrid modified circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide. In some embodiments, the immunogenicity as described herein is assessed by the level of expression or signaling or activation of at least one of RIG-I, TLR-3, TLR-7, TLR-8, MDA-5, LGP-2, OAS, OASL, PKR, and IFN-beta. In some embodiments, the hybrid modified circular polyribonucleotide has a higher half-life than a corresponding unmodified circular polyribonucleotide. In some embodiments, the hybrid modified circular polyribonucleotide has a half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide. In some embodiments, the half-life is measured by introducing the circular polyribonucleotide or the corresponding circular polyribonucleotide into a cell and measuring a level of the introduced circular polyribonucleotide or corresponding circular polyribonucleotide inside the cell.

In some embodiments, the hybrid modified circular polyribonucleotide comprises one or more expression sequences. In some embodiments, the one or more expression sequences of the hybrid modified circular polyribonucleotide has a translation efficiency similar to or higher than a corresponding unmodified circular polyribonucleotide. In some embodiments, the one or more expression sequences of the hybrid modified circular polyribonucleotide have a translation efficiency of that is at least about 0.7, 0.8, 0.9, 1.0, 1.1, 1.2, 1.3, 1.5, 1.6, 1.8, 2, 2.2, 2.5, 2.8, or 3 fold higher than a corresponding unmodified circular polyribonucleotide. In some embodiments, the one or more expression sequences of the hybrid modified circular polyribonucleotide have a higher translation efficiency than a corresponding circular polyribonucleotide having a portion comprising a modified nucleotide (e.g., the portion corresponds to the unmodified portion of the hybrid modified circular polyribonucleotide). In some embodiments, one or more expression sequences of the circular polyribonucleotide are configured to have a higher translation efficiency than a corresponding circular polyribonucleotide having a first portion comprising more than 10%, or at least 20%, 30%, 40%, 50%, 60%, 70%, 80%, 90%, or 100% modified nucleotides. In some embodiments, the one or more expression sequences of the hybrid modified circular polyribonucleotide has a translation efficiency that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding circular polyribonucleotide having a portion comprising a modified nucleotide (e.g., the portion corresponds to the unmodified portion of the hybrid modified circular polyribonucleotide). As described herein, in some embodiments, the translation efficiency is measured either in a cell comprising the circular polyribonucleotide or the corresponding circular polyribonucleotide, or in an in vitro translation system (e.g., rabbit reticulocyte lysate).

In some embodiments, the hybrid modified circular polyribonucleotide has a binding site that is unmodified, e.g., having no modified nucleotides. In some embodiments, the hybrid modified circular polyribonucleotide has a binding site configured to bind to a protein, DNA, RNA, or cell target that is unmodified, e.g., having no modified nucleotides. In some embodiments, the hybrid modified circular polyribonucleotide has an internal ribosome entry site (IRES) that is unmodified, e.g., having no modified nucleotides. In some embodiments, the hybrid modified circular polyribonucleotide has no more than 10% of the nucleotides in the binding site that are modified nucleotides. In some embodiments, the hybrid modified circular polyribonucleotide has no more than 10% of the nucleotides in the binding site configured to bind to a protein, DNA, RNA, or cell target that are modified nucleotides. In some embodiments, the hybrid modified circular polyribonucleotide has no more than 10% of the nucleotides in the internal ribosome entry site (IRES) that are modified nucleotides. In some embodiments, a hybrid modified circular polyribonucleotide has modified nucleotides throughout except the binding site. In some embodiments, a hybrid modified circular polyribonucleotide has modified nucleotides throughout except the binding site configured to bind a protein, DNA, RNA, or a cell target. In some embodiments, a hybrid modified circular polyribonucleotide has modified nucleotides throughout except the IRES element. In other embodiments, the hybrid modified circular polyribonucleotide has modified nucleotides throughout except the IRES element and one or more other portions. Without wishing to be bound by a certain theory, the unmodified IRES element renders the hybrid modified circular polyribonucleotide translation competent, e.g., having a translation efficiency for the one or more expression sequences that is similar to or higher than a corresponding circular polyribonucleotide that does not have any modified nucleotides.

In some embodiments, the hybrid modified circular polyribonucleotide has modified nucleotides, e.g., 5′ methylcytidine and pseudouridine, throughout the circular polyribonucleotide except the IRES element or a binding site configured to bind a protein, DNA, RNA, or a cell target. In these cases, the hybrid modified circular polyribonucleotide has a higher a lower immnogeneicity as compared to a corresponding circular polyribonucleotide that does not comprise 5′ methylcytidine and pseudouridine. In some embodiments, the hybrid modified circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide. In some embodiments, the immunogenicity as described herein is assessed by expression or signaling or activation of at least one of RIG-I, TLR-3, TLR-7, TLR-8, MDA-5, LGP-2, OAS, OASL, PKR, and IFN-beta. In some embodiments, the hybrid modified circular polyribonucleotide has n higher half-life than a corresponding unmodified circular polyribonucleotide, e.g., a corresponding circular polyribonucleotide that does not comprise 5′ methylcytidine and pseudouridine. In some embodiments, the hybrid modified circular polyribonucleotide has a higher half-life that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide. In some embodiments, the half-life is measured by introducing the circular polyribonucleotide or the corresponding circular polyribonucleotide into a cell and measuring a level of the introduced circular polyribonucleotide or corresponding circular polyribonucleotide inside the cell.

In some cases, the hybrid modified circular polyribonucleotide as described herein has similar immunogenicity as compared to a corresponding circular polyribonucleotide that is otherwise the same but completely modified. For instance, a hybrid modified circular polyribonucleotide that has 5′ methylcytidine and pseudouridine throughout except its IRES element can have similar immunogenicity or lower immunogenicity as compared to a corresponding circular polyribonucleotide that is otherwise the same but has 5′ methylcytidine and pseudouridine throughout and no unmodified cytidine and uridine. In some embodiments, the hybrid modified circular polyribonucleotide that has 5′ methylcytidine and pseudouridine throughout except its IRES element has translation efficiency that is similar to or higher than the translation efficiency of a corresponding circular polyribonucleotide that is otherwise the same but has 5′ methylcytidine and pseudouridine throughout and no unmodified cytidine and uridine.

### Conjugation of Circular Polyribonucleotides

A circRNA of the disclosure can be conjugated, for example, to a chemical compound (e.g., a small molecule), an antibody or fragment thereof, a peptide, a protein, an aptamer, a drug, or a combination thereof. In some embodiments, a small molecule can be conjugated to a circRNA, thereby generating a circRNA comprising a small molecule.

A circRNA of the disclosure can comprise a conjugation moiety to facilitate conjugation. A conjugation moiety can be incorporated, for example, at an internal site of a circular polynucleotide, or at a 5′ end, 3′ end, or internal site of a linear polynucleotide. A conjugation moiety can be incorporated chemically or enzymatically. For example, a conjugation moiety can be incorporated during solid phase oligonuleotide synthesis, cotranscriptionally (e.g., with a tolerant RNA polymerase) or posttranscriptionally (e.g., with a RNA methyltransferase). A conjugation moiety can be a modified nucleotide or a nucleotide analog, e.g., bromodeoxyuridine. A conjugation moiety can comprise a reactive group or a functional group, e.g., an azide group or an alkyne group. A conjugation moiety can be capable of undergoing a chemoselective reaction. A conjugation moiety can be a hapten group, e.g., comprising digoxigenin, 2,4-dinitrophenyl, biotin, avidin, or selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A conjugation moiety can comprise a diarylethene photoswitch capable of undergoing reversible electrocyclic rearrangement. A conjugation moiety can comprise a nucleophile, a carbanion, and/or an α,β-unsaturated carbonyl compound.

A circRNA can be conjugated via a chemical reaction, e.g., using click chemistry, Staudinger ligation, Pd-catalyzed C—C bond formation (e.g., Suzuki-Miyaura reaction), Michael addition, olefin metathesis, or inverse electron demand Diels-Alder. Click chemistry can utilize pairs of functional groups that rapidly and selectively react (“click”) with each other in appropriate reaction conditions. Non-limiting click chemistry reactions include azide-alkyne cycloaddition, copper-catalyzed 1,3-dipolar azide-alkyne cycloaddition (CuAAC), strain-promoted Azide-Alkyne Click Chemistry reaction (SPAAC), and tetrazine-alkene Ligation.

Non-limiting examples of functionalized nucleotides include azide modified UTP analogs, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2′,3′-ddATP, 5-Azido-PEG4-CTP, 5-DBCO-PEG4-CTP, N6-Azidohexyl-3′-dATP, 5-DBCO-PEG4-dCpG, and 5-azidopropyl-UTP. In some embodiments, a circRNA comprises at least one 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 5-Azido-PEG4-CTP, 5-DBCO-PEG4-CTP, or 5-azidopropyl-UTP.

A single modified nucleotide of choice (e.g., modified A, C, G, U, or T containing an azide at the 2′-position) can be incorporated site-specifically under optimized conditions (e.g., via solid-phase chemical synthesis). A plurality of nucleotides containing an azide at the 2′-position can be incorporated, for example, by substituting a nucleotide during an in vitro transcription reaction (e.g., substituting UTP for 5-azido-C3-UTP).

A circRNA conjugate can be generated using a copper-catalyzed click reaction, e.g., copper-catalyzed 1,3-dipolar azide-alkyne cycloaddition (CuAAC) of an alkyne-functionalized small molecule and an azide-functionalized polyribonucleic acid. A linear RNA can be conjugated with a small molecule. For example, a linear RNA can be modified at its 3′-end by a poly(A) polymerase with an azido-derivatized nucleotide. The azide can be conjugated to a small molecule via copper-catalyzed or strain-promoted azide-alkyne click reaction, and the linear RNA can be circularized.

A circRNA conjugate can be generated using a Staudinger reaction. For example, a circular RNA comprising an azide-functionalized nucleotide can be conjugated with an alkyne-functionalized small molecule in the presence of triphenylphosphine-3,3′,3″-trisulfonic acid (TPPTS).

A circRNA conjugate can be generated using a Suzuki-Miyaura reaction. For example, a circRNA comprising a halogenated nucleotide analog can be subjected to Suzuki-Miyaura reaction in the presence of a cognate reactive partner. A a circRNA comprising 5-Iodouridine triphosphate (IUTP), for example, can be used in a catalytic system with Pd(OAc)₂ and 2-aminopyrimidine-4,6-diol (ADHP) or dimethylamino-substituted ADHP (DMADHP) to functionalize iodouridine-labeled circRNA in the presence of various boronic acid and ester substrates. In another example, a circRNA comprising 8-bromoguanosine can be reacted with arylboronic acids in the presence of a catalytic system made of Pd(OAc)₂ and a water-soluble triphenylphosphan-3,3′,3″-trisulfonate ligand.

A circRNA conjugate can be generated using Michael addition, for example, via reaction of an an electron-rich Michael Donor with an α,β-unsaturated compound (Michael Acceptor).

### Structure

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure. In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, the circular polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides.

There are 16 possible base-pairings, however of these, six (AU, GU, GC, UA, UG, CG) can form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, a segment having a quasi-helical structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide has one or more segments (e.g., 2, 3, 4, 5, 6, or more) having a quasi-helical structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide has at least one miRNA binding site, at least one lncRNA binding site, and/or at least one tRNA motif.

### Delivery

The circular polyribonucleotide described herein may be included in pharmaceutical compositions with a delivery carrier.

Pharmaceutical compositions described herein can be formulated for example including a pharmaceutical excipient or carrier. A pharmaceutical carrier may be a membrane, lipid bilayer, and/or a polymeric carrier, e.g., a liposome or particle such as a nanoparticle, e.g., a lipid nanoparticle, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate); electroporation or other methods of membrane disruption (e.g., nucleofection), fusion, and viral delivery (e.g., lentivirus, retrovirus, adenovirus, AAV).

The invention is further directed to a host or host cell comprising the circular polyribonucleotide described herein. In some embodiments, the host or host cell is a plant, insect, bacteria, fungus, vertebrate, mammal (e.g., human), or other organism or cell.

In some embodiments, the circular polyribonucleotide is non-immunogenic in the host. In some embodiments, the circular polyribonucleotide has a decreased or fails to produce a response by the host's immune system as compared to the response triggered by a reference compound, e.g., a linear polynucleotide corresponding to the described circular polyribonucleotide, unmodified circular polyribonucleotide, or a circular polyribonucleotide lacking an encryptogen. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., delivered to or administered to) the circular polyribonucleotide. In some embodiments, the host is a mammal, such as a human. The amount of the circular polyribonucleotide, expression product, or both in the host can be measured at any time after administration. In certain embodiments, a time course of host growth in a culture is determined. If the growth is increased or reduced in the presence of the circular polyribonucleotide, the circular polyribonucleotide or expression product or both is identified as being effective in increasing or reducing the growth of the host.

### Methods of Production

In some embodiments, the circular polyribonucleotide includes a deoxyribonucleic acid sequence that is non-naturally occurring and can be produced using recombinant DNA technology or chemical synthesis.

It is within the scope of the invention that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins). DNA molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide can be prepared, for example, by chemical synthesis and enzymatic synthesis. In some embodiments, a linear primary construct or linear mRNA can be cyclized, or concatemerized to create a circular polyribonucleotide described herein. The mechanism of cyclization or concatemerization can occur through methods such as, but not limited to, chemical, enzymatic, or ribozyme catalyzed methods. The newly formed 5′- or 3′-linkage can be an intramolecular linkage or an intermolecular linkage.

### Pharmaceutical Compositions

The present invention includes compositions in combination with one or more pharmaceutically acceptable excipients. Pharmaceutical compositions can optionally comprise one or more additional active substances, e.g., therapeutically and/or prophylactically active substances. Pharmaceutical compositions of the present invention can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005, which is incorporated herein by reference. In one aspect, the invention includes a method of producing the pharmaceutical composition described herein comprising generating the circular polyribonucleotide.

Although the descriptions of pharmaceutical compositions provided herein are principally directed to pharmaceutical compositions which are suitable for administration to humans, it will be understood by the skilled artisan that such compositions are generally suitable for administration to any other animal, e.g., non-human animals and non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

In one aspect, the invention includes a pharmaceutical composition comprising (a) a circular polyribonucleotide comprising a binding site that binds a target, e.g., a RNA, DNA, protein, membrane of a cell, etc.; and (b) a pharmaceutically acceptable carrier or excipient; wherein the target and the circular polyribonucleotide form a complex, wherein the target is a not a microRNA.

In some embodiments, the binding site is a first binding site and the target is a first target. In some embodiments, the circular polyribonucleotide further comprises a second binding site that binds to a second target.

In one aspect, the invention includes a pharmaceutical composition comprising (a) a circular polribonucleotidecomprising: (i) a first binding site that binds a first target; and (ii) a second binding site that binds a second target; and (b) a pharmaceutically acceptable carrier or excipient; wherein the first binding site is different than the second binding site, wherein the first target and the second target are microRNA.

In some embodiments, the first target comprises a first circular polyribonucleotide (circ-RNA)-binding motif. In some embodiments, the second target comprises a second circular polyribonucleotide (circRNA)-binding motif. In some embodiments, the first target, the second target, and the circular polyribonucleotide form a complex. In some embodiments, the first target and second targets interact with each other. In some embodiments, the complex modulates a cellular process when contacted to the cell. In some embodiments, formation of the complex modulates a cellular process when contacted to the cell. In such embodiments, the cellular process is associated with pathogenesis of a disease or condition.

In some embodiments, the circular polyribonucelotide modulates a cellular process associated with the first or second target when contacted to the cell. In some embodiments, the first and second targets interact with each other in the complex. In some embodiments, the cellular process is associated with pathogenesis of a disease or condition. In some embodiments, the cellular process is different than translation of the circular polyribonucleotide. In some embodiments, the first target comprises a deoxyribonucleic acid (DNA) molecule, and thetarget comprises a protein. In some embodiments, the complex modulates directed transcription of the DNA molecule, epigenetic remodeling of the DNA molecule, or degradation of the DNA molecule.

In some embodiments, the first target comprises a first protein, and the second target comprises a second protein. In such embodiments, the complex modulates degradation of the first protein, translocation of the first protein, or signal transduction, or modulates formation of a complex formed by direct interaction between the first and second proteins (e.g., inhibits or promotes formation of a complex).

In some embodiments, the first target comprises a first ribonucleic acid (RNA) molecule, and the second target comprises a second RNA molecule. In such embodiments, the complex can modulate degradation of the first RNA molecule.

In some embodiments, the target comprises a protein, and the second target comprises a RNA molecule. In such embodiments, the complex modulates translocation of the protein or inhibits formation of a complex formed by direct interaction between the protein and the RNA molecule.

In some embodiments, the first target is a receptor, and the second target is a substrate of the receptor. In such embodiments, the complex inhibits activation of the receptor. As used herein, a “receptor” can refer to a protein molecule that receives chemical signals from outside a cell. The chemical signals can include, without limitation, small molecule organic compounds (e.g., amino acids and derivatives thereof, e.g., glutamate, glycine, gamma-butyrateric acid), lipids, protein or polypeptides, DNA and RNA molecules, and ions. A receptor can be present on cell membrane, in cytoplasm, or in cell nucleus. The chemical signals that bind to a receptor can be generally referred to as “substrate” of the receptor. Upon binding to the chemical signal, a receptor can cause some form of cellular response by initiating one or more cellular processes, e.g., signaling pathways. A receptor as provided herein can be any type one skilled in the art would recognize, including: (1) ionotropic receptors, which can be the targets of fast neurotransmitters such as acetylcholine (nicotinic) and GABA; and, activation of these receptors results in changes in ion movement across a membrane. They can have a heteromeric structure in that each subunit consists of the extracellular ligand-binding domain and a transmembrane domain where the transmembrane domain in turn includes four transmembrane alpha helices. The ligand-binding cavities can be located at the interface between the subunits; (2) G protein-coupled receptors, which can include the receptors for several hormones and slow transmitters e.g., dopamine, metabotropic glutamate. They can be composed of seven transmembrane alpha helices. The loops connecting the alpha helices can form extracellular and intracellular domains; (3) kinase-linked and related receptors (or receptor tyrosine kinase), which can be composed of an extracellular domain containing the ligand binding site and an intracellular domain, often with enzymatic-function, linked by a single transmembrane alpha helix. Insulin receptor is an example of this type of receptor, of which insulin can be its corresponding substrate; (4) https://en.wikipedia.org/wiki/Nuclear_receptor nuclear receptors, which can be located in either nucleus, or in the cytoplasm and migrate to the nucleus after binding with their ligands. They can be composed of a C-terminal ligand-binding region, a core DNA-binding domain (DBD) and an N-terminal domain that contains the AF1 (activation function 1) region. Steroid and thyroid-hormone receptors are examples of such receptors, and their corresponding substrates can include various steroids and hormones.

In one aspect, the invention includes a pharmaceutical composition comprising (a) a circular polyribonucleotide comprising a binding site that binds a target; and (b) a pharmaceutically acceptable carrier or excipient; wherein the circular polyribonucleotide is translation incompetent or translation defective, wherein the target is not a microRNA.

In one aspect, the invention includes a pharmaceutical composition comprising (a) a circular polyribonucleotide comprising a binding site that binds a target, wherein the target comprises a first ribonucleic acid (RNA)-binding motif; and (b) a pharmaceutically acceptable carrier or excipient; wherein the circular polyribonucleotide is translation incompetent or translation defective, wherein the target is a microRNA.

In such embodiments, target comprises a DNA molecule. In such embodiments, binding of the target to the circular polyribonucleotide modulates interference of transcription of the DNA molecule. In such embodiments, the target comprises a protein. In such embodiments, binding of target to the circular polyribonucleotide inhibits interaction of the protein with other molecules. In such embodiments, the protein is a receptor, and binding of the target to the circular polyribonucleotide activates the receptor. In such embodiments, the protein is a first enzyme, the circular polyribonucleotide further comprises a second binding site that binds to a second enzyme, and binding of the first and second enzymes to the circular polyribonucleotide modulates enzymatic activity of the first and second enzymes. In such embodiments, the target comprises a messenger RNA (mRNA) molecule. In such embodiments, binding of the target to the circular polyribonucleotide modulates interference of translation of the mRNA molecule. In such embodiments, the target comprises a ribosome. In such embodiments, binding of the target to the circular polyribonucleotide modulates interference of a translation process. In such embodiments, the target comprises a circular RNA molecule. In such embodiments, binding of the target to the circular polyribonucleotide sequesters the circular RNA molecule. In such embodiments, binding of the target to the circular polyribonucleotide sequesters the target.

In one aspect, the invention includes a pharmaceutical composition comprising (a) a circular polyribonucleotide comprising a binding site that binds a cell membrane of a target cell; and wherein the cell membrane of a target cell comprises a first ribonucleic acid (RNA)-binding motif; and (b) a pharmaceutically acceptable carrier or excipient.

In some embodiments, the circular polyribonucleic acid further comprises a second binding site that binds a second membrane of a second target cell, wherein the second cell membrane of the second target cell comprises a second RNA-binding motif. In some embodiments, the circular polyribonucleotide binds to both the cell membrane on the target cell and the second cell membrane of the second target cell, and cellular fusion of the first and second target cells is modulated.

In some embodiments, the circular polyribonucleotide further comprises a second binding site that binds a second target, and binding of both the first and targets to the circular polyribonucleotide induces a conformational change in the first target, thereby inducing signal transduction downstream of the first target in the first cell. In some embodiments, the circular polyribonucleotide is translation incompetent or translation defective.

In some embodiments, the circular polyribonucleic acid further comprises at least one structural element selected from: a) an encryptogen; b) a splicing element; c) a regulatory sequence; d) a replication sequence; e) quasi-double-stranded secondary structure; and f) expression sequence. In such embodiments, the quasi-helical structure comprises at least one double-stranded RNA segment with at least one non-double-stranded segment. In such embodiments, the quasi-helical structure comprises a first sequence and a second sequence linked with a repetitive sequence, e.g., an A-rich sequence. In some embodiments, the encryptogen comprises a splicing element.

In some embodiments, the circular polyribonucleic acid comprises at least one modified nucleic acid. In such embodiments, the at least one modified nucleic acid is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA), a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. The circular polyribonucleotides can be completely modified circular polyribonucleotides. In some embodiments, the administered circular polyribonucleotides are hybrid modified circular polyribonucleotides. In some embodiments, the circular polyribonucleotide comprises modified nucleotides and an unmodified IRES.

In some embodiments, the encryptogen comprises at least one modified nucleic acid, e.g., pseudo-uridine and N(6)methyladenosine (m6A). In some embodiments, the encryptogen comprises a protein binding site, e.g., a ribonucleic acid binding protein. In some embodiments, the encryptogen comprises an immunoprotein binding site, e.g., to evade CTL responses.

In some embodiments, the circular polyribonucleic acid has at least 2× lower immunogenicity than a counterpart lacking the encryptogen, as assessed by expression or signaling or activation of at least one of RIG-I, TLR-3, TLR-7, TLR-8, MDA-5, LGP-2, OAS, OASL, PKR, and IFN-beta. In some embodiments, the circular polyribonucleic acid has a size in the range of about 20 bases to about 20 kb. In some embodiments, the circular polyribonucleic acid is synthesized through circularization of a linear polynucleotide. In some embodiments, the circular polyribonucleic acid is substantially resistant to degradation.

### Applications

Circular polyribonucleotides described herein can be administered to a cell, tissue or subject in need thereof, e.g., to modulate cellular function or a cellular process, e.g., gene expression in the cell, tissue or subject. The invention also contemplates methods of modulating cellular function or a cellular process, e.g., gene expression, comprising administering to a cell, tissue or subject in need thereof a circular polyribonucleotide described herein. The administered circular polyribonucleotides can be modified circular polyribonucleotides. In some embodiments, the administered circular polyribonucleotides are completely modified circular polyribonucleotides. In some embodiments, the administered circular polyribonucleotides are hybrid modified circular polyribonucleotides. In other embodiments, the administered circular polyribonucleotides are unmodified circular polyribonucleotides.

### Embodiment Paragraphs

[1] A pharmaceutical composition comprising:
   (a) a circular polyribonucleotide comprising a binding site that binds a target, e.g., a RNA, DNA, protein, membrane of cell etc.; and
   (b) a pharmaceutically acceptable carrier or excipient;
   wherein the target and the circular polyribonucleotide form a complex, and
   wherein the target is a not a microRNA.
[2] A pharmaceutical composition comprising:
   (a) a circular polyribonucleotide comprising:
      (i) a first binding site that binds a first target, and
      (ii) a second binding site that binds a second target; and
   (b) a pharmaceutically acceptable carrier or excipient;
   wherein the first binding site is different than the second binding site, and
   wherein the first target and the second target are both a microRNA.
[3] The pharmaceutical composition of paragraph [1], wherein the binding site comprises an aptamer sequence.
[4] The pharmaceutical composition of paragraph [2], wherein the first binding site comprises a first aptamer sequence and the second binding site comprises a second aptamer sequence.
[5] The pharmaceutical composition of claim [3], wherein the aptamer sequence has a secondary structure that binds the target.
[6] The pharmaceutical composition of claim [4], wherein the first aptamer sequence has a secondary structure that binds the first target and the second aptamer sequence has a secondary structure that binds the second target.
[7] The pharmaceutical composition of claim [1], wherein the binding site is a first binding site and the target is a first target.
[8] The pharmaceutical composition of any one of paragraphs [3], [5], and [7], wherein the circular polyribonucleotide further comprises a second binding site that binds to a second target.
[9] The pharmaceutical composition of any one of paragraphs [2], [4], [6], [7], and [8], wherein the first target comprises a first circular polyribonucleotide (circRNA)-binding motif.
[10] The pharmaceutical composition of any one of paragraphs [2], [4], [6], and [7]-[9], wherein the second target comprises a second circular polyribonucleotide (circRNA)-binding motif.
[11] The pharmaceutical composition of any one of paragraphs [2], [4], [6], and [7]-[10], wherein the first target, the second target, and the circular polyribonucleotide form a complex.
[12] The pharmaceutical composition of any one of paragraphs [2], [4], [6], and [7]-[11], wherein the first and second targets interact with each other.
[13] The pharmaceutical composition of any one of paragraphs [1], [3], [5], and [7]-[12], wherein the complex modulates a cellular process.
[14] The pharmaceutical composition of any one of paragraphs [2], [4], [6], and [7]-[13], wherein the first and second targets are the same, and the first and second binding sites bind different binding sites on the first target and the second target.
[15] The pharmaceutical composition of any one of paragraphs [2], [4], [6], and [7]-[13], wherein the first target and the second target are different.
[16] The pharmaceutical composition of any one of paragraphs [2], [4], [6], and [7]-[15], wherein the circular polyribonucleotide further comprises one or more additional binding sites that bind a third or more targets.
[17] The pharmaceutical composition of any one of paragraphs [2], [4], [6], and [7]-[16], wherein one or more targets are the same and one or more additional binding sites bind different binding sites on the one or more targets.
[18] The pharmaceutical composition of any one of paragraphs [1], [3], [5], and [7]-[17], wherein formation of the complex modulates a cellular process.
[19] The pharmaceutical composition of any one of paragraphs [2], [4], [6], and [7]-[18], wherein the circular polyribonucleotide modulates a cellular process associated with the first or second target when contacted to the first and second targets.
[20] The pharmaceutical composition any one of paragraphs [2], [4], [6], and [7]-[19], wherein the first and second targets interact with each other in the complex.
[21] The pharmaceutical composition of any one of paragraphs [13]-[20], wherein the cellular process is associated with pathogenesis of a disease or condition.
[22] The pharmaceutical composition of any one of paragraphs [13]-[21], wherein the cellular process is different than translation of the circular polyribonucleic acid.
[23] The pharmaceutical composition of any one of paragraphs [2], [4], [6], and [7]-[22], wherein the first target comprises a deoxyribonucleic acid (DNA) molecule, and the second target comprises a protein.
[24] The pharmaceutical composition of any one of paragraphs [1], [3], [5], and [7]-[23], wherein the complex modulates directed transcription of the DNA molecule, epigenetic remodeling of the DNA molecule, or degradation of the DNA molecule.
[25] The pharmaceutical composition of any one of paragraphs [2], [4], [6], and [7]-[24], wherein the first target comprises a first protein, and the second target comprises a second protein.
[26] The pharmaceutical composition of any one of paragraphs [1], [3], [5], and [7]-[25], wherein the complex modulates degradation of the first protein, translocation of the first protein, or signal transduction, or modulates a native protein function, inhibits or modulates formation of a complex formed by direct interaction between the first and second proteins.
[27] The pharmaceutical composition of any one of paragraphs [2], [4], [6], and [7]-[26], wherein the first target or the second target is a ubiquitin ligase.
[28] The pharmaceutical composition of any one of paragraphs [2], [4], [6], and [7]-[27], wherein the first target comprises a first ribonucleic acid (RNA) molecule, and the second target comprises a second RNA molecule.
[29] The pharmaceutical composition of paragraph [28], wherein the complex modulates degradation of the first RNA molecule.
[30] The pharmaceutical composition of any one of paragraphs [2], [4], [6], and [7]-[29], wherein the first target comprises a protein, and the second target comprises a RNA molecule.
[31] The pharmaceutical composition of any one of paragraphs [1], [3], [5], and [7]-[30], wherein the complex modulates translocation of the protein or inhibits formation of a complex formed by direct interaction between the protein and the RNA molecule.
[32] The pharmaceutical composition of any one of paragraphs [2], [4], [6], and [7]-[31], wherein the first target is a receptor, and the second target is a substrate of the receptor.
[33] The pharmaceutical composition of any one paragraphs [1], [3], [5], and [7]-[32], wherein the complex inhibits activation of the receptor.
[34] A pharmaceutical composition comprising:
   (a) a circular polyribonucleotide comprising a binding site that binds a target; and
   (b) a pharmaceutically acceptable carrier or excipient;
   wherein the circular polyribonucleotide is translation incompetent or translation defective, and wherein the target is not a microRNA.
[35] A pharmaceutical composition comprising:
   (a) a circular polyribonucleic acid comprising a binding site that binds a target, wherein the target comprises a ribonucleic acid (RNA)-binding motif; and
   (b) a pharmaceutically acceptable carrier or excipient;
   wherein the circular polyribonucleotide is translation incompetent or translation defective, and wherein the target is a microRNA.
[36] The pharmaceutical composition of any of one paragraphs [34] and [35], wherein the binding site comprises an aptamer sequence having a secondary structure that binds the target.
[37] The pharmaceutical composition of any one of paragraphs [34] and [36], wherein the target comprises a DNA molecule.
[38] The pharmaceutical composition of any one of paragraphs [34]-[37], wherein binding of the target to the circular polyribonucleotide modulates interference of transcription of a DNA molecule.
[39] The pharmaceutical composition of any one of paragraphs [34] and [36]-[38], wherein the target comprises a protein.
[40] The pharmaceutical composition of paragraph [39], wherein binding of the target to the circular polyribonucleotide modulates interaction of the protein with other molecules.
[41] The pharmaceutical composition of any one of paragraphs [39]-[40], wherein the protein is a receptor, and wherein binding of the target to the circular polyribonucleotide activates the receptor.
[42] The pharmaceutical composition of any one of paragraphs [39]-[41], wherein the protein is a first enzyme, wherein the circular polyribonucleotide further comprises a second binding site that binds to a second enzyme, and wherein binding of the first and second enzymes to the circular polyribonucleotide modulates enzymatic activity of the first and second enzymes.
[43] The pharmaceutical composition of any one of paragraphs [39] and [40], wherein the protein is a ubiquitin ligase.
[44] The pharmaceutical composition of any one of paragraphs [34], [36], and [38], wherein the target comprises a messenger RNA (mRNA) molecule.
[45] The pharmaceutical composition of paragraph [44], wherein binding of the target to the circular polyribonucleotide modulates interference of translation of the mRNA molecule.
[46] The pharmaceutical composition of any one of the paragraphs [34], [36], [39], and [40], wherein the target comprises a ribosome.
[47] The pharmaceutical composition of any one of paragraphs [34]-[46], wherein binding of the target to the circular polyribonucleotide modulates interference of a translation process.
[48] The pharmaceutical composition of any one of paragraphs [34], [36], and [38], wherein the target comprises a circular RNA molecule.
[49] The pharmaceutical composition of paragraph [48], wherein binding of the target to the circular polyribonucleotide sequesters the circular RNA molecule.
[50] The pharmaceutical composition of any one of the paragraphs [35], [36], [38], and [47], wherein binding of the target to the circular polyribonucleotide sequesters the microRNA molecule.
[51] A pharmaceutical composition comprising:
   (a) a circular polyribonucleotide comprising a binding site that binds to a membrane of a cell (e.g., cell wall membrane, organelle membrane, etc.), wherein the membrane of the cell comprises a ribonucleic acid (RNA)-binding motif; and
   (b) a pharmaceutically acceptable carrier or excipient.
[52] The pharmaceutical composition of paragraph [51], wherein the binding site comprises an aptamer sequence having a secondary structure that binds the membrane of the cell (e.g., cell wall membrane, organelle membrane, etc.).
[53] The pharmaceutical composition of any one of paragraphs [51] and [52], wherein the circular polyribonucleotide further comprises a second binding site that binds to a second target, wherein the second target comprises a second RNA-binding motif.
[54] The pharmaceutical composition of paragraph [53], wherein the circular polyribonucleotide binds to the membrane of the cell and the second target.
[55] The pharmaceutical composition of any one of paragraphs [51]-[54], wherein the circular polyribonucleotide further comprises a second binding site that binds to a second cell target, and wherein binding of the cell target and the second cell target to the circular polyribonucleotide induces a conformational change in the cell target, thereby inducing signal transduction downstream of the cell target.
[56] The pharmaceutical composition of any one of paragraphs [1]-[55], wherein the circular polyribonucleotide is translation incompetent or translation defective.
[57] The pharmaceutical composition of any one of paragraphs [1]-[56], wherein the circular polyribonucleotide further comprises at least one structural element selected from the group consisting of:
   a) an encryptogen;
   b) a splicing element;
   c) a regulatory sequence;
   d) a replication sequence;
   e) a quasi-double-stranded secondary structure
   f) a quasi-helical structure; and
   g) an expression sequence.
[58] The pharmaceutical composition of paragraph [57], wherein the quasi-helical structure comprises at least one double-stranded RNA segment with at least one non-double-stranded segment.
[59] The pharmaceutical composition of any one of paragraphs [57] and [58], wherein the quasi-helical structure comprises a first sequence and a second sequence linked with a repetitive sequence.
[60] The pharmaceutical composition of any one paragraphs [57]-[59], wherein the encryptogen comprises a splicing element.
[61] The pharmaceutical composition of any one of paragraphs [1]-[60], wherein the circular polyribonucleic acid comprises at least one modified nucleic acid.
[62] The pharmaceutical composition of paragraph [61], wherein the at least one modified nucleic acid is selected from the group consisting of 2′-O-methyl, 2′-O-methoxyethyl (2′-0-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA), a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.
[63] The pharmaceutical composition of any one of paragraphs [57]-[62], wherein the encryptogen comprises at least one modified nucleic acid.
[64] The pharmaceutical composition of any one of paragraphs [57]-[63], wherein the encryptogen comprises a protein binding site.
[65] The pharmaceutical composition of any one of paragraphs [57]-[64], wherein the encryptogen comprises an immunoprotein binding site.
[66] The pharmaceutical composition of any one of paragraphs [57]-[65], wherein the circular polyribonucleic acid has at least 2× lower immunogenicity than a counterpart lacking the encryptogen, as assessed by expression, signaling, or activation of at least one of RIG-I, TLR-3, TLR-7, TLR-8, MDA-5, LGP-2, OAS, OASL, PKR, and IFN-beta.
[67] The pharmaceutical composition of any one of paragraphs [1]-[66], wherein the circular polyribonucleic acid has a size of about 20 bases to about 20 kb.
[68] The pharmaceutical composition of any one of paragraphs [1]-[67], wherein the circular polyribonucleic acid is synthesized through circularization of a linear polynucleotide.
[69] The pharmaceutical composition of any one of paragraphs [1]-[68], wherein the circular polyribonucleic acid is substantially resistant to degradation.
[70] A pharmaceutical composition, comprising:
   (a) a circular polyribonucleotide comprising a binding site that binds to a target, wherein the target comprises a ribonucleic acid (RNA)-binding motif; and
   (b) a pharmaceutically acceptable carrier or excipient,
   wherein the circular polyribonucleotide comprises at least one modified nucleotide and a first portion that comprises at least about 5, 10, 20, 50, 100, 200, 300, 400, 500, 600, 700, 800, 900, or 1000 contiguous unmodified nucleotides.
[71] A pharmaceutical composition, comprising:
   (a) a circular polyribonucleotide comprising a binding site that binds to a target, wherein the target comprises a ribonucleic acid (RNA)-binding motif; and
   (b) a pharmaceutically acceptable carrier or excipient, wherein the circular polyribonucleotide comprises at least one modified nucleotide and a first portion that comprises at least about 5, 10, 20, 50, 100, 200, 300, 400, 500, 600, 700, 800, 900, or 1000 contiguous nucleotides, and wherein the first portion lacks pseudouridine or 5′-methylcytidine.
[72] The pharmaceutical composition of any one of paragraphs [70] and [71], wherein the binding site comprises an aptamer sequence having a secondary structure that binds the target.
[73] The pharmaceutical composition of any one of paragraphs [70]-[72], wherein the circular polyribonucleotide has a lower immunogenicity than a corresponding unmodified circular polyribonucleotide.
[74] The pharmaceutical composition of any one of paragraphs [70]-[72], wherein the circular polyribonucleotide has an immunogenicity that is at least about 1.1, 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold lower than a corresponding unmodified circular polyribonucleotide, as assessed by expression or signaling or activation of at least one of the group consisting of RIG-I, TLR-3, TLR-7, TLR-8, MDA-5, LGP-2, OAS, OASL, PKR, and IFN-beta.
[75] The pharmaceutical composition of any one of paragraphs [70]-[74], wherein the circular polyribonucleotide has a higher half-life than a corresponding unmodified circular polyribonucleotide.
[76] The pharmaceutical composition of any one of paragraphs [70]-[74], wherein the circular polyribonucleotide has a half-life that is at least about 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding unmodified circular polyribonucleotide.
[77] The pharmaceutical composition of any one of paragraphs [75] and [76], wherein the half-life is measured by introducing the circular polyribonucleotide or the corresponding unmodified circular polyribonucleotide into a cell and measuring a level of the introduced circular polyribonucleotide or corresponding circular polyribonucleotide inside the cell.
[78] The pharmaceutical composition of any one of paragraphs [70]-[77], wherein the at least one modified nucleotide is selected from the group consisting of: N(6)methyladenosine (m6A), 5′-methylcytidine, and pseudouridine.
[79] The pharmaceutical composition of any one of paragraphs 70-[77], wherein the at least one modified nucleic acid is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA), a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.
[80] The pharmaceutical composition of any one of paragraphs [70]-[79], wherein at least about 10%, 20%, 30%, 40%, 50%, 60%, 70%, 80%, 90%, 95%, or 99% of nucleotides of the circular polyribonucleotide are modified nucleotides.
[81] The pharmaceutical composition of any one of paragraphs [70]-[80], wherein the circular polyribonucleotide comprises a binding site that binds to a protein, DNA, RNA, or a cell target, consisting of unmodified nucleotides.
[82] The pharmaceutical composition of any one of paragraphs [70]-[81], wherein the circular polyribonucleotide comprises an internal ribosome entry site (IRES) consisting of unmodified nucleotides.
[83] The pharmaceutical composition of any one of paragraphs [70]-[80], wherein the binding site consists of unmodified nucleotides.
[84] The pharmaceutical composition of paragraph [83], wherein the binding site comprises an IRES consisting of unmodified nucleotides.
[85] The pharmaceutical composition of any one of paragraphs [70]-[84], wherein the first portion comprises a binding site that binds a protein, DNA, RNA, or a cell target.
[86] The pharmaceutical composition of any one of paragraphs [70]-[85], wherein the the first portion comprises an IRES.
[87] The pharmaceutical composition of any one of paragraphs [70]-[86], wherein the circular polyribonucleotide comprises one or more expression sequences.
[88] The pharmaceutical composition of any one of paragraphs [82]-[87], wherein the circular polyribonucleotide comprises the one or more expression sequences and the IRES, and wherein the circular polyribonucleotide comprises a 5′-methylcytidine, a pseudouridine, or a combination thereof outside the IRES.
[89] The pharmaceutical composition of any one of paragraphs [70]-[88], wherein one or more expression sequences of the circular polyribonucleotide are configured to have a higher translation efficiency than a corresponding unmodified circular polyribonucleotide.
[90] The pharmaceutical composition of any one of paragraphs [70]-[89], wherein one or more expression sequences of the circular polyribonucleotide have a translation efficiency of that is at least about 0.7, 0.8, 0.9, 1.0, 1.1, 1.2, 1.3, 1.5, 1.6, 1.8, 2, 2.2, 2.5, 2.8, or 3 fold higher than a corresponding unmodified circular polyribonucleotide.
[91] The pharmaceutical composition of any one of paragraphs [70]-[90], wherein one or more expression sequences of the circular polyribonucleotide have a higher translation efficiency than a corresponding circular polyribonucleotide having a first portion comprising a modified nucleotide.
[92] The pharmaceutical composition of any one of paragraphs [70]-[90], wherein one or more expression sequences of the circular polyribonucleotide have a higher translation efficiency than a corresponding circular polyribonucleotide having a first portion comprising more than 10% modified nucleotides.
[93] The pharmaceutical composition of any one of paragraphs [70]-[92], wherein one or more expression sequences of the circular polyribonucleotide have a translation efficiency that is at least about 1.2, 1.3, 1.5, 1.6, 1.8, 2, 2.2, 2.5, 2.8, 3, 3.2, 3.3, 3.5, 3.8, 4.0, 4.2, 4.5, 4.8, 5.0, 5.5, 6.0, 6.5, 7.0, 7.5, 8.0, 8.5, 9.0, 9.5, or 10.0 fold higher than a corresponding circular polyribonucleotide having a first portion comprising a modified nucleotide.
[94] The pharmaceutical composition of any one of paragraphs [89]-[93], wherein the translation efficiency is measured either in a cell comprising the circular polyribonucleotide or the corresponding circular polyribonucleotide, or in an in vitro translation system (e.g., rabbit reticulocyte lysate).
[95] The pharmaceutical composition of any one of paragraphs [70]-[94], wherein the circular polyribonucleotide is the circular polyribonucleotide of any one of claims 0-[69].
   [96] A method of treatment, comprising administering the pharmaceutical composition of any one of paragraphs [1]-[95] to a subject with a disease or condition.
[97] A method of producing a pharmaceutical composition, comprising generating the circular polyribonucleotide of any one of paragraphs [1]-[95].
   [98] The circular polyribonucleotide of any one of the paragraphs [1]-[95] formulated in a carrier, e.g., membrane or lipid bilayer.
[99] A method of making the circular polyribonucleotide of any one of paragraphs [1]-[95], comprising circularizing a linear polyribonucleotide having a nucleic acid sequence as the circular polyribonucleotide.
[100] An engineered cell comprising the composition of any one of claims [1]-[95].
[101] A method of binding a target in a cell, the method comprising:
   providing a translation incompetent circular polyribonucleotide comprising an aptamer sequence, wherein the aptamer sequence has a secondary structure that binds the target; and
   delivering the translation incompetent circular polyribonucleotide to the cell, wherein the translation incompetent circular polyribonucleotide forms a complex with the target detectable at least 5 days after delivery.
[102] A method of binding a target in a cell, the method comprising:
   delivering a translation incompetent circular polyribonucleotide to the cell, wherein the translation incompetent circular polyribonucleotide comprises an aptamer sequence that binds the target, and wherein the translation incompetent circular polyribonucleotide forms a complex with the target detectable at least 5 days after delivery.
[103] The method of any one of the paragraphs [101] and [102], wherein the target is selected from the group consisting of a nucleic acid molecule, a small molecule, a protein, a carbohydrate, and a lipid.
[104] The method of any one of paragraphs [101]-[103], wherein the target is a gene regulation protein.
[105] The method of paragraph 104, wherein the gene regulation protein is a transcription factor.
[106] The method of paragraph [103], wherein the nucleic acid molecule is a DNA molecule or an RNA molecule.
[107] The method of any one of paragraphs [101]-[106], wherein the complex modulates gene expression.
[108] The method of any one of paragraphs [101]-[107], wherein the complex modulates directed transcription of a DNA molecule, epigenetic remodeling of a DNA molecule, or degradation of DNA molecule.
[109] The method of any one of paragraphs [101]-[108], wherein the complex modulates degradation of the target, translocation of the target, or target signal transduction.
[110] The method of any one of paragraphs [107]-[109], wherein the gene expression is associated with pathogenesis of a disease or condition.
[111] The method of any one of paragraphs [101]-[110], wherein the complex is detectable at least 7, 8, 9, or 10 days after delivery.
[112] The method of any one of paragraphs [101]-[111], wherein the translation incompetent circular polyribonucleotide is present at least five days after the delivering.
[113] The method of any one of paragraphs [101]-[112], wherein the translation incompetent circular polyribonucleotide is present at least 6, 7, 8, 9, or 10 days after the delivering
[114] The method of any one of paragraphs [101]-[113], wherein the translation incompetent circular polyribonucleotide is an unmodified translation incompetent circular polyribonucleotide.
[115] The method of any one of paragraphs [101]-[114], wherein the translation incompetent circular polyribonucleotide has a quasi-double-stranded secondary structure.
[116] The method of any one of paragraphs [101]-[115], wherein the aptamer sequence further has a tertiary structure that binds the target.
[117] The method of any one of paragraphs [101]-[116], wherein the cell is a eukaryotic cell.
[118] The method any one paragraph [117], wherein the eukaryotic cell is a human cell.
[119] A method of binding a transcription factor in a cell, the method comprising:
   providing a translation incompetent circular polyribonucleotide comprising an aptamer sequence that binds the transcription factor; and
   delivering the translation incompetent circular polyribonucleotide to the cell, wherein the translation incompetent circular polyribonucleotide forms a complex with the transcription factor and modulates gene expression.
[120] A method of binding a transcription factor in a cell, the method comprising:
   delivering a translation incompetent circular polyribonucleotide to the cell, wherein the translation incompetent circular polyribonucleotide comprises an aptamer sequence that binds the transcription factor, and wherein the translation incompetent circular polyribonucleotide forms a complex with the transcription factor and modulates gene expression.
[121] A method of sequestering a transcription factor in a cell, the method comprising:
   providing a translation incompetent circular polyribonucleotide comprising an aptamer sequence that binds the transcription factor; and
   delivering the translation incompetent circular polyribonucleotide to the cell, wherein the translation incompetent circular polyribonucleotide sequesters the transcription factor by binding the transcription factor to form a complex in the cell.
[122] A method of sequestering a transcription factor in a cell, the method comprising:
   delivering a translation incompetent circular polyribonucleotide to the cell, wherein the translation incompetent circular polyribonucleotide comprises an aptamer sequence that binds the transcription factor, and wherein the translation incompetent circular polyribonucleotide sequesters the transcription factor by binding the transcription factor to form a complex.
[123] The method of any one of paragraphs [121] and [122], wherein cell viability decreases after formation of the complex.
[124] A method of sensitizing a cell to a cytotoxic agent, the method comprising:
   providing a translation incompetent circular polyribonucleotide comprising an aptamer sequence that binds a transcription factor; and
   delivering the cytotoxic agent and the translation incompetent circular polyribonucleotide to the cell, wherein the translation incompetent circular polyribonucleotide forms a complex with the transcription factor in the cell;
   thereby sensitizing the cell to the cytotoxic agent compared to a cell lacking the translation incompetent circular polyribonucleotide.
[125] A method of sensitizing a cell to a cytotoxic agent, the method comprising:
   delivering the cytotoxic agent and a translation incompetent circular polyribonucleotide to the cell, wherein the translation incompetent circular polyribonucleotide comprises an aptamer sequence that binds the transcription factor; and wherein the translation incompetent circular polyribonucleotide forms a complex with the transcription factor in the cell;
   thereby sensitizing the cell to the cytotoxic agent compared to a cell lacking the translation incompetent circular polyribonucleotide.
[126] The method of any one of paragraphs [124] and [125], wherein the sensitizing the cell to the cytotoxic agent results in decreased cell viability after the delivering of the cytotoxic agent and the translation incompetent circular polyribonucleotide.
[127] The method of paragraph [126], wherein the decreased cell viability is decreased by 40% or more at least two days after the delivering of the cytotoxic agent and the translation incompetent circular polyribonucleotide.
[128] A method of binding a pathogenic protein in a cell, the method comprising:
   providing a translation incompetent circular polyribonucleotide comprising an aptamer sequence that binds the pathogenic protein; and
   delivering the translation incompetent circular polyribonucleotide to the cell, wherein the translation incompetent circular polyribonucleotide forms a complex with the pathogenic protein for degrading the pathogenic protein.
[129] A method of binding a pathogenic protein in a cell, the method comprising:
   delivering a translation incompetent circular polyribonucleotide to the cell, wherein the translation incompetent circular polyribonucleotide comprises an aptamer sequence that binds the pathogenic protein; and wherein the translation incompetent circular polyribonucleotide forms a complex with the pathogenic protein for degrading the pathogenic protein.
[130] A method of binding a ribonucleic acid molecule in a cell, the method comprising:
   providing a translation incompetent circular polyribonucleotide comprising a sequence complementary to a sequence of the ribonucleic acid molecule; and
   delivering the translation incompetent circular polyribonucleotide to the cell, wherein the translation incompetent circular polyribonucleotide forms a complex with the ribonucleic acid molecule in the cell.
[131] A method of binding a ribonucleic acid molecule in a cell, the method comprising:
   delivering a translation incompetent circular polyribonucleotide to the cell, wherein the translation incompetent circular polyribonucleotide comprises an aptamer sequence that binds the ribonucleic acid molecule; wherein the translation incompetent circular polyribonucleotide forms a complex with the ribonucleic acid molecule in the cell.
[132] A method of binding genomic deoxyribonucleic acid molecule in a cell, the method comprising:
   providing a translation incompetent circular polyribonucleotide comprising an aptamer sequence that binds the genomic deoxyribonucleic acid molecule; and delivering the translation incompetent circular polyribonucleotide to the cell, wherein the translation incompetent circular polyribonucleotide forms a complex with the genomic deoxyribonucleic acid molecule and modulates gene expression.
[133] A method of binding genomic deoxyribonucleic acid molecule in a cell, the method comprising:
   delivering a translation incompetent circular polyribonucleotide to the cell, wherein the translation incompetent circular polyribonucleotide comprises an aptamer sequence that binds the genomic deoxyribonucleic acid molecule; wherein the translation incompetent circular polyribonucleotide forms a complex with the genomic deoxyribonucleic acid molecule and modulates gene expression.
[134] A method of binding a small molecule in a cell, the method comprising:
   providing a translation incompetent circular polyribonucleotide comprising an aptamer sequence that binds the small molecule; and
   delivering the translation incompetent circular polyribonucleotide to the cell, wherein the translation incompetent circular polyribonucleotide forms a complex with the small molecule and modulates a cellular process (e.g., protein degradation, cell signaling, gene expression, etc.).
[135] A method of binding a small molecule in a cell, the method comprising:
   delivering a translation incompetent circular polyribonucleotide to the cell, wherein the translation incompetent circular polyribonucleotide comprises an aptamer sequence that binds the small molecule; wherein the translation incompetent circular polyribonucleotide forms a complex with the small molecule and modulates a cellular process (e.g., protein degradation, cell signaling, gene expression, etc.).
[136] The method of any one of paragraphs [134] and [135], wherein the small molecule is an organic compound with a molecular weight of no more than 900 daltons and modulates a cellular process.
[137] The method of any one of paragraphs [134]-[136], wherein the small molecule is a drug.
[138] The method of any one of paragraphs [134] and [135], wherein the small molecule is a fluorophore.
[139] The method of any one of paragraphs [134]-[136], wherein the small molecule is a metabolite.
[140] A composition comprising a translation incompetent circular polyribonucleotide comprising an aptamer sequence, wherein the aptamer sequence has a secondary structure that binds a target.
[141] A pharmaceutical composition comprising a translation incompetent circular polyribonucleotide comprising an aptamer sequence, wherein the aptamer sequence has a secondary structure that binds the target; and a pharmaceutically acceptable carrier or excipient.
[142] A cell comprising the translation incompetent circular polyribonucleotide of any one of paragraphs [101]-[141].
[143] A method of treating a subject in need thereof, comprising administering the composition of any one of paragraphs [101]-[140] or the pharmaceutical composition of paragraph [141].
[144] A polynucleotide encoding the translation incompetent circular polyribonucleotide of any one of paragraphs [101]-[141].
[145] A method of producing the translation incompetent circular polyribonucleotide of any one of paragraphs [101]-[141].

All references and publications cited herein are hereby incorporated by reference.

The following examples are provided to further illustrate some embodiments of the present invention, for example using model elements, but are not intended to limit the scope of the invention; it will be understood by their exemplary nature that other procedures, methodologies, or techniques known to those skilled in the art can alternatively be used.

### EXAMPLES

### Example 1: Circular RNA that Binds DNA to Regulate Gene Expression

This Example describes circular RNA binding to DNA to regulate gene expression.

A non-naturally occurring circular RNA is engineered to include a sequence within a model target gene, in this case, the dihydrofolate reductase (DHFR) gene. Found in all organisms, DHFR plays a critical role in regulating the amount of tetrahydrofolate in the cell. Tetrahydrofolate and its derivatives are essential for purine and thymidylate synthesis, which are important for cell proliferation and cell growth. DHFR plays a central role in the synthesis of nucleic acid precursors. As shown in the following Example, the circular RNA binds to the DHFR gene to suppress its transcription.

Circular RNA is designed to include the DHFR binding sequence 5′-ACAAAUGGGGACGAGGGGGGCGGGGCGGCC-3′ (SEQ ID NO: 5).

Unmodified linear RNA is synthesized by in vitro transcription using T7 RNA polymerase from a DNA segment including the DHFR binding sequence described above. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

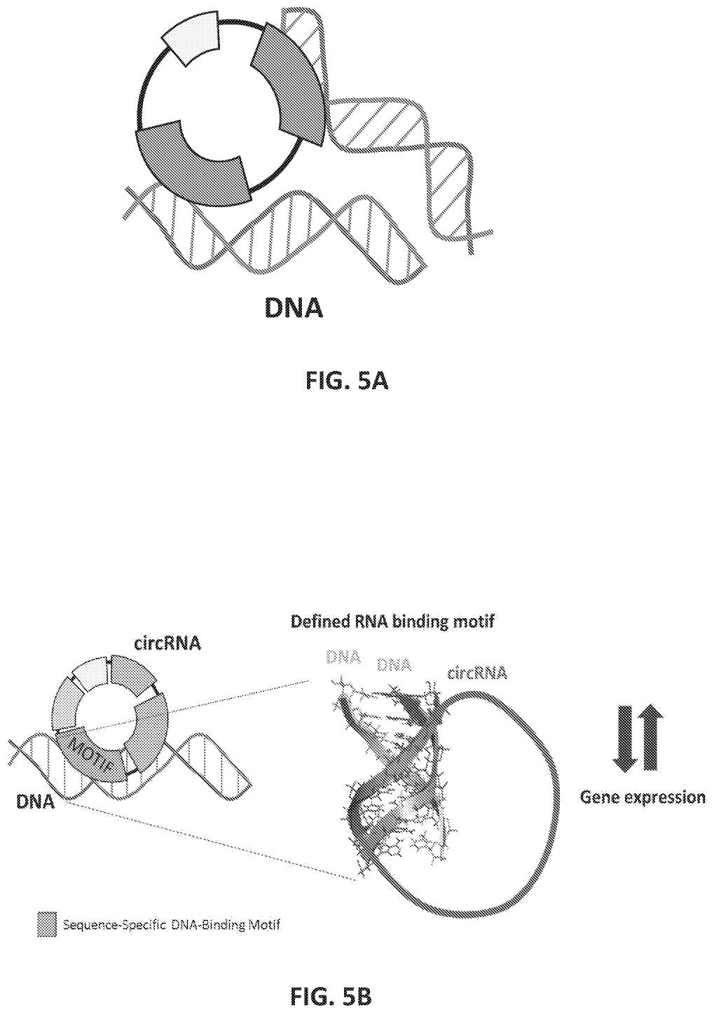

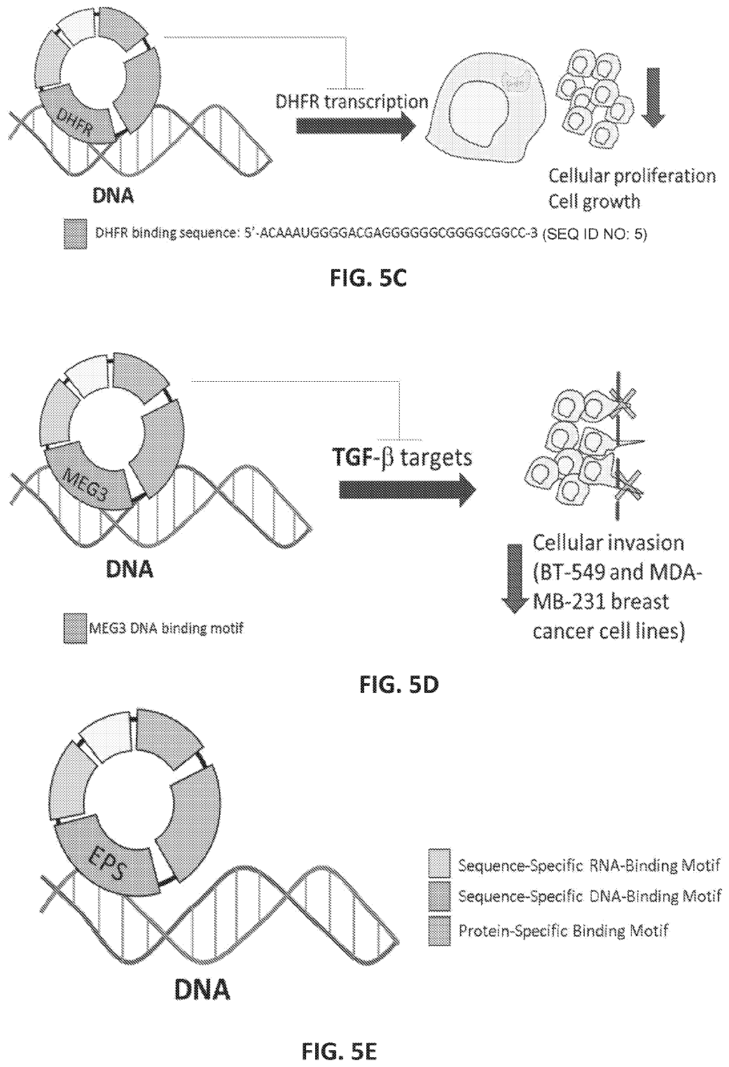

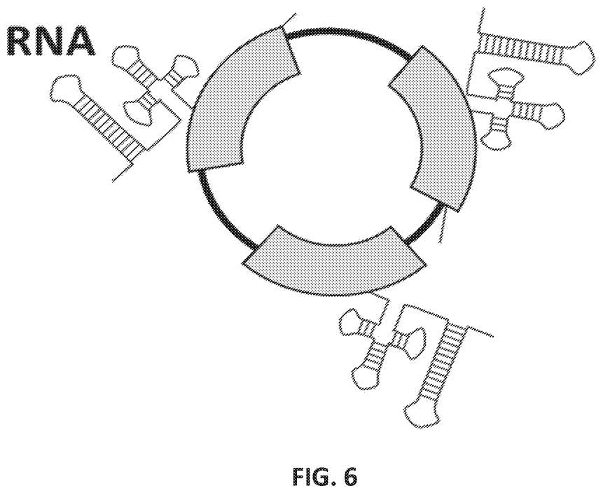

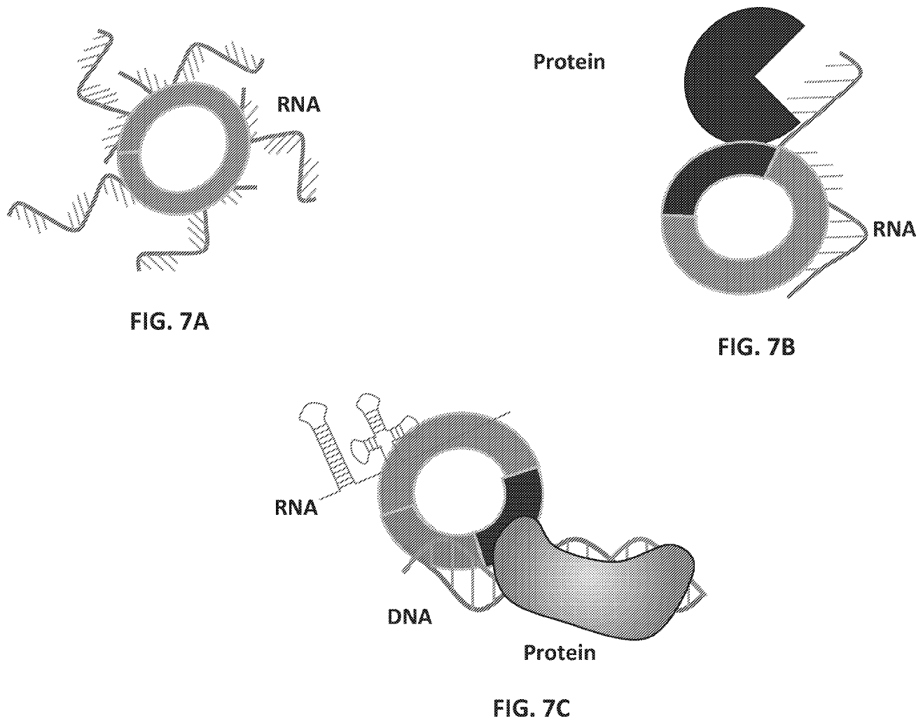

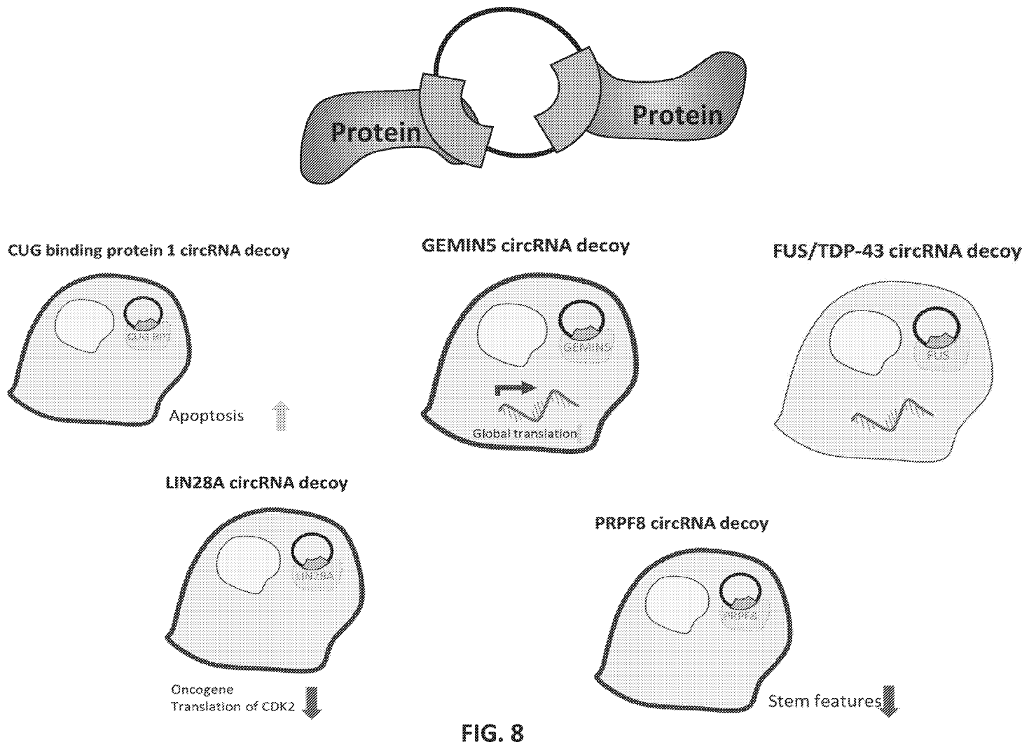

As shown in FIG. 5C, one circular RNA binding to the DHFR genomic DNA is assessed through several methods including CHART-qPCR, which evaluates direct RNA binding to the genomic DNA, DHFR transcript specific qPCR, as well as cellular proliferation and cell growth assays. Active binding of circular RNA to the DHFR gene is expected to result in decreased DHFR transcription, a decrease in purine and thymidylate synthesis, and decreased cell proliferation and cell growth.

### Example 2: Circular RNA that Binds dsDNA to Regulate Gene Expression

This Example describes circular RNA binding to dsDNA to regulate gene expression.

As shown in FIG. 5D, a non-naturally occurring circular RNA is engineered to include a sequence that binds to a model target gene, in this case, transforming growth factor beta (TGF-β) target sequences. TGF-β is secreted by many cell types. After binding to the TGF-β receptor, the receptor phosphorylates and activates a signaling cascade that leads to the activation of different downstream substrates and regulatory proteins. The following Example describes the circular RNA binding to the TGF-β target genes to suppress their transcription.

Circular RNA is designed to include the TGF-β target binding sequence 5′-CGGAGAGCAGAGAGGGAGCG-3′ (SEQ ID NO: 6).

Unmodified linear RNA is synthesized by in vitro transcription using T7 RNA polymerase from a DNA segment having the TGF-β binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to dsDNA is evaluated through a triplex immune capture assay. Here, the formation of RNA-DNA triple structures is assessed using a biotin-labelled Triplex Forming Oligonucleotide (TFO) ssRNA molecule (either control sequence or targeting sequence 5′-CGGAGAGCAGAGAGGGAGCG-3′ (SEQ ID NO: 7)) to pull down target DNA sequences from within cells or from nuclei isolated from cells. DNA pulled down by the biotinylated targeting or control TFOs are sequenced to determine DNA sequences enriched following RNA-dsDNA pulldown.

Alternative methods to demonstrate RNA-DNA binding include CHART-qPCR and gel mobility shift assay where the targeting ssRNA oligo (5′-CGGAGAGCAGAGAGGGAGCG-3′ (SEQ ID NO: 7)) interacts with the target dsDNA oligo (5′-AGAGAGAGGGAGAGAG-3′ (SEQ ID NO: 8) and 3′-TCTCTCTCCCTCTCTC-5′ (SEQ ID NO: 9)) but not control DNA oligos.

Additional assessments for functional changes induced following target RNA binding include changes in TGF-β target genes, including TGFB2, TGFBR1 and/or SMAD2, measured by qPCR.

### Example 3: Circular RNA that Binds DNA to Regulate Gene Expression

This Example describes circular RNA binding to DNA to inhibit transcription factor binding.

A non-naturally occurring circular RNA is engineered to include a binding sequence to a target sequence, here a gamma globin transcription factor binding sequence. Fetal hemoglobin is the main oxygen transport protein in the human fetus during the last seven months of development in the uterus and persists in the newborn until roughly 6 months after birth. Fetal hemoglobin binds oxygen with greater affinity than adult hemoglobin, giving the developing fetus better access to oxygen from the mother's bloodstream. In newborns, fetal hemoglobin is nearly completely replaced by adult hemoglobin by approximately 6 months postnatally.

GATA-1 is a constituent of the repressor complex GATA-1-FOG-1-Mi2b that binds at the −567^{G}γ/-566^{A}γ-globin GATA motifs. The following Example describes circular RNA binding to the −567^{G}γ/-566^{A}γ-globin GATA motifs (GenBank coordinates 33992 to 33945 from accession file GI455025 and GenBank coordinates 38772 to 38937 from accession file GI455025, respectively) to prevent inhibitory transcription factors/repressive complexes from binding.

Circular RNA is designed to include the non-deletional binding sequence where inhibitory transcription factor complex GATA1, Mi2b or FOG1, binds.

Unmodified linear RNA is synthesized by in vitro transcription using T7 RNA polymerase from a DNA segment having the transcription factor binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to DNA is assessed through a direct DNA binding method like CHART-qPCR and function is assessed through methods like the activation and expression of fetal hemoglobin. Active binding of circular RNA to regulatory elements upstream of the γ-globin genes is expected to result in competitive inhibition of the transcription factor, BCL11A, or other inhibitory transcription factors to activate HbF transcription. Changes in HbF levels may be measured through HPLC analysis, flow cytometric analysis, and/or qPCR.

### Example 4: Circular RNA that Binds a DNA Duplex

This Example describes circular RNA binding to a DNA duplex.

A non-naturally occurring circular RNA can be engineered to include a DNA binding sequence to the major groove. Short (15-mer) RNA oligonucleotides (triplex forming oligonucleotide (TFO)) can form a stable triple helical RNA:DNA complex. The third strand in the triplex structure (i.e. the TFO) follows a path through the major groove of the duplex DNA. The specificity and stability of the triplex structure is afforded via Hoogsteen hydrogen bonds, which are different from those formed in classical Watson-Crick base pairing in duplex DNA. The TFO binds to the purine-rich strand of the target duplex through the major groove.

Unmodified linear RNA is synthesized by in vitro transcription using T7 RNA polymerase from a DNA segment having polypurine sequence of 10-15 bases. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to DNA is assessed through a direct DNA binding method, such as CHART-qPCR, which evaluates direct RNA binding to the genomic DNA. Alternative methods to evaluate circular RNA binding to dsDNA include a triplex immune capture assay and gel mobility shift assay.

### Example 5: Circular RNA that Binds and Sequesters RNA Transcripts

This Example describes circular RNA binding to and sequestering RNA transcripts.

A non-naturally occurring circular RNA is engineered to include one or more novel binding sequences for RNA transcripts. RNA molecules with expanded CGG tracts are targeted for circular RNA binding. As shown in the following Example, the circular RNA binds to the repeat region of the RNA for sequestration.

Circular RNA is designed to include the complementary sequence to 50-220 FMR1 expansion repeats 5′-CGG-3′.

Unmodified linear RNA is synthesized by in vitro transcription using T7 RNA polymerase from a DNA segment having the 50-220 FMR1 expansion repeats. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to FMR1 mRNA is evaluated by an oligonucleotide pull-down-qPCR assay, in which modified oligonucleotides complementary to the circular RNA are used to pull-down the FMR1 mRNA, which is reverse transcribed and qPCR amplified. Binding is also assessed by colocalization of two fluorescent oligos, one specific for the FMR1 mRNA and one complementary to the circular RNA and evaluation by RNA FISH.

### Example 6: Circular RNA that Binds and Sequesters RNA Transcripts

This Example describes circular RNA binding to and sequestering RNA transcripts.

A non-naturally occurring circular RNA is engineered to include one or more novel binding sequences for RNA transcripts. SCA8 utilizes an expansion repeat of CTG. The CTG repeat occurs in a gene that is transcribed but not translated. As shown in the following Example, the circular RNA binds to the repeat region of the mRNA for sequestration.

Circular RNA is designed to include the complementary sequence to 50-120 SCA8 expansion repeats 5′-CUG-3′.

Unmodified linear RNA is synthesized by in vitro transcription using T7 RNA polymerase from a DNA segment having the 50-120 SCA8 expansion repeats. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to SCA1 RNA is evaluated by an oligonucleotide pull-down-qPCR assay, in which modified oligonucleotides complementary to the circular RNA are used to pull-down the SCA8 expansion repeats, which are reverse transcribed and qPCR amplified. RNA FISH is also used to asses-binding by colocalization of two fluorescent oligos, one specific for the SCA8 RNA and one complementary to the circular RNA is evaluated by RNA FISH.

### Example 7: Circular RNA that Binds and Sequesters RNA Transcripts

This Example describes circular RNA binding to and sequestering RNA transcripts.

A synthetic circular RNA is engineered to include one or more novel binding sequences for RNA transcripts. The huntingtin (HTT) gene includes a segment of 6-35 glutamine residues in its wild-type form. As shown in the following Example, the circular RNA binds to the repeat region of the mRNA for sequestration.

Circular RNA is designed to include the complementary sequence to 40-120 HTT expansion repeats 5′-CAG-3′.

Unmodified linear RNA is synthesized by in vitro transcription using T7 RNA polymerase from a DNA segment having the 40-120 HTT expansion repeats. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to HTT RNA is evaluated by an oligonucleotide pull-down-qPCR assay, in which modified oligonucleotides complementary to the circular RNA are used to pull-down the HTT RNA, which are reverse transcribed and qPCR amplified. RNA FISH is also used to asses-binding by colocalization of two fluorescent oligos, one specific for the HTTA and one complementary to the circular RNA is evaluated by RNA FISH.

### Example 8: Circular RNA that Binds and Sequesters RNA Transcripts and Enzyme

This Example describes circular RNA simultaneously binding to and sequestering RNA transcripts and protein to aid in RNA degradation.

A non-naturally occurring circular RNA is engineered to include one or more novel binding sequences for transcripts as well as a protein to aid in transcript degradation. The atrophin-1 protein is encoded by the ATN1 and is used as a model system. The encoded protein includes a serine repeat, a region of alternating acidic and basic amino acids, as well as the variable glutamine repeat. ATN1 gene has a segment of DNA called the CAG trinucleotide repeat.

In eukaryotic cells, most mRNAs have a 5′ monomethyl guanosine cap structure and a 3′ poly(A) tail which are important for mRNA translation and stability. Removal of the 5′cap structure (decapping) is a prerequisite for decay of the mRNA body from the 5′ end. The Dcp2 protein has been identified as the major mRNA decapping enzyme in cells. As shown in the following Example, the circular RNA binds to the repeat region of the mRNA for sequestration and Dcp2 protein for decapping of the mRNA.

Circular RNA is designed to include the complementary sequence to 40-120 ATN1 expansion repeats 5′-CAG-3′ and RNA cap structure for recognition by Dcp2.

Unmodified linear RNA is synthesized by in vitro transcription using T7 RNA polymerase from a DNA segment having the 40-120 ATN1 expansion repeats and RNA cap structure for recognition by Dcp2. Transcribed RNA is purified with an RNA purification system (QIAGEN), treated with alkaline phosphatase (ThermoFisher Scientific, EF0652) following the manufacturer's instructions, and purified again with the RNA purification system. Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to ATN1 RNA is evaluated by an oligonucleotide pull-down-qPCR assay, in which modified oligonucleotides complementary to the circular RNA are used to pull-down the ATN1 RNA, which are reverse transcribed and qPCR amplified. Decapping function is evaluated by qSL-RT-PCR, which combines splinted ligation and quantitative RT-PCR (Blewett, et al., RNA, 2011, Mar. 17(3): 535-543).

### Example 9: Circular RNA for mRNA Replacement

This Example describes circular RNA binding to a target mRNA, creating a ribozyme cleavage site.

A non-naturally occurring circular RNA is engineered to include a sequence that binds to the M2 isoform of pyruvate kinase mRNA. As shown in the following Example, the circular RNA binds to the target M2 isoform of pyruvate kinase (PK), resulting in its cleavage.

Circular RNA is designed to include sequences complementary to the M2 isoform of pyruvate kinase that will generate a VS ribozyme cleavage site in the target. Circular RNA additionally includes sequences for the trans-acting VS ribozyme and the coding sequence for the M1 isoform of pyruvate kinase.

Unmodified linear RNA is synthesized by in vitro transcription using T7 RNA polymerase from a DNA segment having the M2 isoform complementary sequence, VS ribozyme, and M1 coding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to, and concomitant degradation of, PK M2 mRNA is evaluated by RT-PCR. Restored expression of PK M1 mRNA is evaluated in a similar manner. Additionally, expression of PK M1 and PK M2 proteins is evaluated by western blotting. Evidence for functional changes induced following target RNA binding and cleavage include cell proliferation assays.

### Example 10: Circular RNA for Targeted mRNA Cleavage

This Example describes circular RNA binding to a model target mRNA, creating a ribozyme cleavage site.

A non-naturally occurring circular RNA is engineered to include a sequence that binds to the SRSF1 mRNA The following Example describes the circular RNA binding to the target SRSF1 mRNA, resulting in its cleavage.

Circular RNA is designed to include sequences complementary to tSRSF1 mRNA that will generate a VS ribozyme cleavage site in the target. Circular RNA additionally contains sequences for the trans-acting VS ribozyme and the coding sequence for the M1 isoform of pyruvate kinase. Other trans-acting ribozymes, such as HDV, hammerhead, group I, and/or group II, are utilized.

Unmodified linear RNA is synthesized by in vitro transcription using T7 RNA polymerase from a DNA segment having SRSF1 complementary sequence, VS ribozyme. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to, and concomitant degradation of, SRSF1 mRNA is evaluated by RT-PCR. Expression of SRSF1 protein is evaluated by western blotting. Additional evidence for changes induced following target RNA binding and cleavage include cell proliferation assays.

### Example 11: Circular RNA that Sequesters Circular RNA

This Example describes circular RNA binding circular RNA.

Circular RNA may be present in certain cell lines. One such example is circ-Dnmt1. As shown in the following Example, the circular RNA binds to circ-Dnmt1.

A circular RNA is designed to include a complementary sequence to circ-Dnmt1 to inhibit its RNA-protein interactions.

Unmodified linear RNA is synthesized by in vitro transcription using T7 RNA polymerase from a DNA segment having the appropriate sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to circ-Dnmt1 is by pull-down of circular RNA using a biotinylated oligo complementary to a region of the circular RNA followed by RT-PCR. Additionally, electrophoretic mobility shift assay is used to visualize circular RNA-circDnmt1 complexes.

### Example 12: Circular RNA that Sequesters Two miRNA

This Example describes circular RNA binding two separate miRNAs.

A circular RNA is designed to include a complementary sequence to two model miRNAs, here miR-9 and miR-1269.

Unmodified linear RNA is synthesized by in vitro transcription using T7 RNA polymerase from a DNA segment having the appropriate sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to miR-9 and miR-1269 is by pull-down of circular RNA using a biotinylated oligo complementary to a region of the circular RNA followed by RT-PCR. Additionally, electrophoretic mobility shift assay is used to visualize circular RNA-miRNA-miRNA complexes.

### Example 13: Circular RNA that Binds and Sequesters at Least Two Individual RNA Transcripts

This Example describes circular RNA binding to and sequestering at least two model RNA transcripts.

A synthetic circular RNA is engineered to include two or more novel binding sequences for RNA transcripts. SCA8 utilizes an expansion repeat of CTG. The FMR1 gene includes CGG expansions. As shown in the following Example, the circular RNA binds to the repeat region of RNA transcripts for sequestration.

As shown in the following Example, the circular RNA binds to the repeat region of the RNA for sequestration of either the FMR1 or SCA8 expansion repeats.

Circular RNA is designed to include the complementary sequence to 50-220 FMR1 expansion repeats 5′-CGG-3′ and the complementary sequence to 50-120 SCA8 expansion repeats 5′-CUG-3′.

Unmodified linear RNA is synthesized by in vitro transcription using T7 RNA polymerase from a DNA segment having the expansion repeats. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to FMR1 or SCA1 mRNA is evaluated by an oligonucleotide pull-down-qPCR assay, in which modified oligonucleotides complementary to the circular RNA are used to pull-down the FMR1 or SCA1 mRNA, which is reverse transcribed and qPCR amplified. Binding is also assessed by colocalization of fluorescent oligos, one specific for the FMR1 or SCA1 mRNA and one complementary to the circular RNA and fluorescence is evaluated by RNA FISH.

### Example 14: Circular RNA that Binds Protein

This Example describes circular RNA binding to protein for sequestration.

TAR-DNA binding protein-43 (TDP-43) is a multifunctional heterogeneous ribonucleoprotein implicated in mRNA processing and stabilization. TDP-43 comprises two RNA recognition motifs (RRMs), a nuclear localization signal and a nuclear export sequence mediating nuclear shuttling, as well as a C-terminal glycine-rich domain (GRD) implicated in TDP-43 protein interactions and functions. As shown in the following Example, the circular RNA binds to TDP-43 for sequestration.

Circular RNA is designed to include the TDP-43 RNA binding motifs: 5′-(UG)nUA(UG)m-3′, 5′-GAGAGAGCGCGUGUGUGUGUGUGGUGGUGCAUA-3′ (SEQ ID NO: 10) or (UG)₆ and a protein binding sequence for the C-terminal glycine-rich domain to competitively bind TDP-43 and inhibit its binding/downstream functions.

Unmodified linear RNA is synthesized by in vitro transcription using T7 RNA polymerase from a DNA segment comprising the TDP-43 RNA motif and protein binding sequence for the C-terminal glycine-rich domain. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to TDP-43 is evaluated in vitro by EMSA (RNA electrophoretic mobility shift assay). When TDP-43 is bound to circRNA migration speed during the gel electrophesis is slower than that of unbound circular RNA. Also, RIP (RNA immunoprecipitation) using anti-TDP-43 antibody, coupled with circular RNA specific qPCR is used to evaluate transcript binding in cellular extracts. To asses-if circular RNA binds to TDP-43 for sequestration, TDP-43 localization is analyzed in cells treated with and without circular RNA. If circular RNA sequesters TDP-43, TDP-43 localization is expected to remain in the cytoplasm. Additionally, in TDP43 sequestration by circular RNA is expected to result in increased survival.

### Example 15: Circular RNA that Binds Protein

This Example describes circular RNA binding to protein for sequestration.

Pre-mRNA-processing-splicing factor 8 is a protein that in humans is encoded by the PRPF8 gene and is a component of both U2- and U12-dependent spliceosomes, and found to be essential for the catalytic step II in pre-mRNA splicing process. As shown in the following Example, the circular RNA binds to PRPF8 for sequestration.

Circular RNA is designed to include at the PRPF8 RNA binding motif 5′-AUUGCCUAUAGAACUUAUAACGAACAUGGUUCUUGCCUUUUACCAGAACCAUCC GGGUGUUGUCUCCAUAGA-3′ (SEQ ID NO: 11) to competitively bind PRPF8 and inhibit its function.

Unmodified linear RNA is synthesized by in vitro transcription using T7 RNA polymerase from a DNA segment comprising PRPF8 binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to PRPF8 is EMSA (RNA electrophoretic mobility shift assay). When PRPF8 is bound to circular RNA, migration speed during the gel electrophesis is slower than that of unbound circular RNA. Also, RIP (RNA immunoprecipitation) using anti-PRPF8 antibody, coupled with circular RNA specific qPCR is used to evaluate transcript binding in cellular extracts. To asses if circular RNA sequesters PRPF8 and alters cell function, the expression of stem cell surface markers like CD44+/CD24+ is evaluated by FACS after circular RNA delivery.

### Example 16: Circular RNA that Binds Protein

This Example describes circular RNA binding to a model protein for sequestration.

The human LIN28A homolog is an RNA binding protein (RBP) with an N-terminal cold-shock domain (CSD) and two C-terminal CysCysHisCys (CCHC) zinc finger domains. Human LIN28A is predominantly cytoplasmic and associates with cellular components, such as ribosomes, P-bodies, and stress granules. As shown in the following Example, the circular RNA binds to LIN28A for sequestration.

Circular RNA is designed to include the preE_{M}-let-7f sequence, 5′-GGGGUAGUGAUUUUACCCUGGAGAU-3′ (SEQ ID NO: 12), an RNA sequence with the LIN28A GGAG binding motif to competitively bind LIN28A.

Unmodified linear RNA is synthesized by in vitro transcription using T7 RNA polymerase from a DNA segment comprising a LIN28A binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to LIN28A is EMSA (RNA electrophoretic mobility shift assay). When LIN28A is bound to circular RNA, migration speed during the gel electrophesis is slower than that of unbound circular RNA. Also, RIP (RNA immunoprecipitation) using anti-LIN28A antibody, coupled with circular RNA specific qPCR is used to evaluate transcript binding in cellular extracts and a combined LIN28A-immunofluorescence with circular RNA FISH is used to evaluate colocalization in cells. To asses if circular RNA binds to LIN28A for sequestration and altered cell function, circular RNA is delivered into human cells. Upon circular RNA treatment, expression levels of mature LET-7g are measured by q-RT-PCR. In addition, cell growth of treated cells is measured by the MTT method.

### Example 17: Circular RNA that Binds Protein

This Example describes circular RNA binding to a model protein for sequestration.

CUG-binding protein 1 (CUGBP1) regulates gene expression at the levels of alternative splicing, mRNA degradation, and translation. Posttranscriptional regulatory network involves the RNA-binding protein CUG-binding protein 1 (CUGBP1), also referred to as CUGBP- and ELAV-like family member 1 (CELF1), which binds to a GU-rich element (GRE) residing in the 3′-UTR of target transcripts and mediates degradation of GRE-containing transcripts. As shown in the following Example, the circular RNA binds to CUGBP1 for sequestration.

Circular RNA is designed to include at least one RNA motif having UGU(G/U)UGU(G/U)UGU that is recognized by CUGBP1 and competitively bind CUGBP1.

Unmodified linear RNA is synthesized by in vitro transcription using T7 RNA polymerase from a DNA segment ‘comprising CUGBP1 binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to CUGBP1 is EMSA (RNA electrophoretic mobility shift assay). When CUGBP1 is bound to circular RNA, migration speed during the gel electrophesis is slower than that of unbound circular RNA. Also, RIP (RNA immunoprecipitation) using anti-CUGP1 antibody, coupled with circular RNA specific qPCR is used to evaluate transcript binding in cellular extracts and a combined CUGP1-immunofluorescence with circular RNA FISH is used to evaluate colocalization in cells. To assess if circular RNA binds to CUGBP1 for sequestration and altered cell function, circular RNA is delivered into cells and cell proliferation can be as measured using a colorimetric MTT assay.

### Example 18: Circular RNA that Binds Protein

This Example describes circular RNA binding to a model protein for sequestration.

Gemin5 is a RNA-binding protein (RBP) is a predominantly cytoplasmic protein with a C-terminal domain harboring a non-canonical bipartite RNA-binding site consisting of RBS1 and RBS2 domains. Additionally, Gemin5 binds the 7-methylguanosine (m7G) cap present in RNA Polymerase II transcripts and downregulates internal ribosome entry site-dependent translation. Gemin5 may control global protein synthesis through its direct binding to the ribosome by acting as a platform, serving as a hub for distinct RNA-protein networks. The following Example describes the circular RNA binding to GEMIN5 for sequestration.

Circular RNA is designed to include the domain 5 of the Foot and Mouth Disease Virus (FMDV) IRES sequence and competitively bind GEMIN5.

Unmodified linear RNA is synthesized by in vitro transcription using T7 RNA polymerase from a DNA segment comprising GEMIN5 binding sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to GEMIN5 is EMSA (RNA electrophoretic mobility shift assay). When GEMIN5 is bound to circular RNA, migration speed during the gel electrophesis is slower than that of unbound circular RNA. Also, RIP (RNA immunoprecipitation) using anti-GEMIN5 antibody, coupled with circular RNA specific qPCR is used to evaluate transcript binding in cellular extracts and a combined GEMIN5-immunofluorescence with circular RNA FISH is used to evaluate colocalization in cells. To asses if circular RNA sequesters GEMIN5 and alters translation, circular RNA is added to an in vitro translation assay. Translation of a circular RNA encoding a luciferase with an FMDV IRES is measured in the presence and absence of GEMIN5 protein with and without the circular RNA. GEMIN5 sequestration's effect on translation mediated by GEMIN5 protein, as measured by luminescent readout.

### Example 19: Circular RNA that Binds Two Proteins

This Example describes circular RNA simultaneously binding to two model proteins.

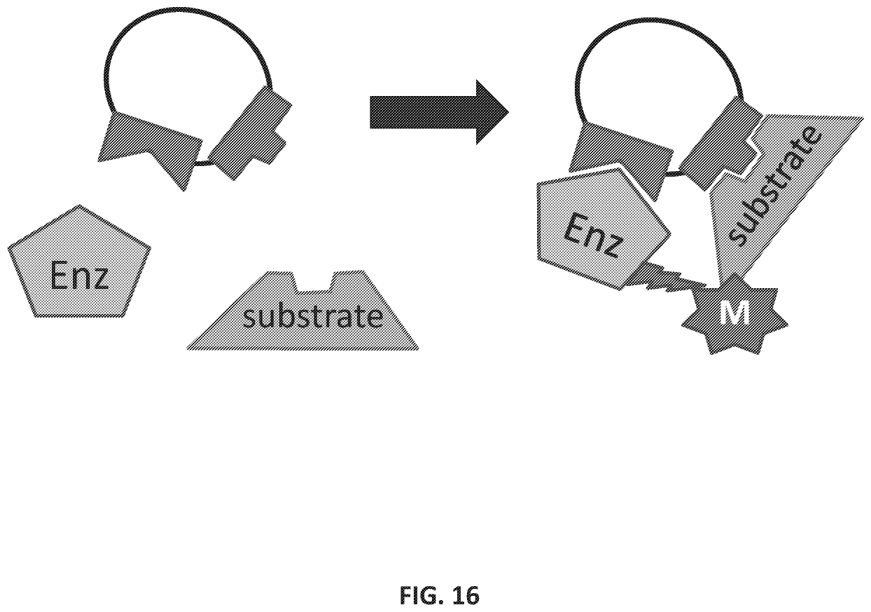

The E3 ubiquitin ligase, MDM2, binds and ubiquitinates proteins, such as p53, marking them for degradation by the proteasome. The following example describes the circular RNA simultaneously binding to MDM2 and p53 to enhance the MDM2-dependent ubiquitination of p53, as illustrated in FIG. 16.

Circular RNA is designed to include the sequence of FOX3 RNA that binds MDM2 and p53.

Unmodified linear RNA is synthesized by in vitro transcription using T7 RNA polymerase from a DNA segment having the appropriate sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to MDM2 and p53 is by electrophoretic mobility shift assay to visualize each RNA-protein complex or alternatively by pull-down of circular RNA using a biotinylated oligo complementary to a region of the circular RNA followed by immunoblotting. Additionally, MDM2 ubiquitination of p53 through binding of circular RNA is assayed via immunoblotting with anti-ubiquitin antibodies or by mass-spectrometry.

### Example 20: Circular RNA that Binds DNA and Protein

This Example describes circular RNA simultaneously binding to DNA and a model protein, here CBP/p300.

CBP/p300 proteins associate with enhancer regions through interactions with eRNAs. RNA binding by CBP/p300 in turn enhances CBP's histone acetyl transferase (HAT) activity. Additionally, CBP and p300 associate with other HATs as well as transcription factors and components of the transcription machinery.

Circular RNA is designed to include the CBP/p300-binding region of eMdm2 eRNA as well as a region complementary to a target genomic locus.

Unmodified linear RNA is synthesized by in vitro transcription using T7 RNA polymerase from a DNA segment having the appropriate sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to CBP/p300 and DNA is pull-down of circular RNA using a biotinylated oligo complementary to a region of the circular RNA, followed by immunoblot and PCR. Additionally, electrophoretic mobility shift assay is used to visualize circular RNA-protein-DNA complexes. Chromatin immunoprecipiration (ChIP) with anti-H3K27ac is performed to detect changes in histone acetylation at the locus of interest and detect binding between the circular RNA, CBP, and the genomic region of interest. Additionally, enhanced expression from a silent genomic locus is assayed via qPCR, or northern/western blot.

### Example 21: Circular RNA that Binds Viral mRNA and miRNA

This Example describes circular RNA simultaneously binding to viral mRNA and miRNA.

Herpes simplex virus-1 (HSV-1) encodes multiple miRNAs regulating viral transcription. HSV-1-miR-H27 bnids mRNA of the host transcriptional regulator Kelch-like 24 (KLHL24) to induce transcription of viral immediate early and early genes.

Circular RNA is designed to include the complementary sequences to HSV-1 miR-H27 and KLHL24.

Unmodified linear RNA is synthesized by in vitro transcription using T7 RNA polymerase from a DNA segment having the appropriate sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to both transcripts is by pull-down of circular RNA using a biotinylated oligo complementary to a region of the circular RNA followed by RT-PCR. Additionally, electrophoretic mobility shift assay can be used to visualize circular RNA-mRNA-miRNA complexes.

### Example 22: Circular RNA that Binds a Lipid Membrane

This Example describes circular RNA binding to a lipid membrane.

Circular RNA can be designed to specifically bind to lipid membranes. The following Example describes a circular RNA binding to a membrane. By mediating binding of cellular membranes, circular RNA is able to bring adjacent cells into close proximity of one another.

Circular RNA is designed to include at least one RNA motif (sequences described herein) that is designed to bind a membrane:

(SEQ ID NO: 13)
GUGAUGGCGCCUACGUCGAAGAAAGGAGUCUCAAGGGAAGGAGCGUAUAUG
GUCGAUGAAUCGGUCAUGUCGUCAGGGU;
(SEQ ID NO: 14)
GAGUCAUAGGACGCUCGCUCUUGCGACCAUGGGGCACGGGGAGCCCACUGC
AUGGAUCU AUCGUAU CAUAGUGCGGU;
(SEQ ID NO: 15)
GUAGCUUCCAUGAGACUUGAUCGGGGUCAUGGCUCUAGGCAUCGGAGAAGC
UGACUAACU UGGUCACGUCGUACCUGGU;
(SEQ ID NO: 16)
GGACGCGUACGAAGGGCUGAUAGGGCAGAGCUCCAACUAUGCGUCCAGCUC
GUGCAGUGGAUCGGGUCGUGCCUGGU;
and
(SEQ ID NO: 17)
CUUUGUCGGCCGAACUCGCUGUUUAACUGCCCGGCGAGAUCGCAGGGUGUU
GUGCUAUU CGCGUGCCGUGUG.

Unmodified linear RNA is synthesized by in vitro transcription using T7 RNA polymerase from a DNA segment comprising one or more of the RNA lipid binding motifs. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

One method to assess circular RNA binding to a lipid membrane is incubation of the circular RNAs with liposomes. Liposomes are fractionated using a Sephacryl S-1000 column. All unbound RNA is discarded. Bound circular RNA is assessed through qPCR, or northern blotting.

### Example 23: Circular RNA for siRNA Delivery

This Example describes circular RNA delivering several siRNAs.

A non-naturally occurring circular RNA is engineered to include siRNA sequences that bind to the model target Transthyretin (TTR) mRNA. The following Example describes the circular RNA derived siRNAs binding to the target TTR mRNA to inhibit of transthyretin protein translation.

Circular RNA is designed to include sequences complementary to TTR mRNA (e.g. auggaauacu cuugguuactt), which bind to transthyretin mRNA resulting in the cleavage of this mRNA.

Unmodified linear RNA is synthesized by in vitro transcription using T7 RNA polymerase from a DNA segment having TTR complementary sequence. Transcribed RNA is purified with an RNA purification system (QIAGEN), treated with alkaline phosphatase (ThermoFisher Scientific, EF0652) following the manufacturer's instructions, and purified again with the RNA purification system.

To generate circular RNA, the two RNA ends, bearing a 5′-phosphate and 3′-OH are designed with additional flanking complementary sequences. These complementary sequences hybridize, resulting in a nicked circle. This nick is closed by T4 DNA ligase. Circular RNA quality is assessed by agarose or PAGE gel, or through automated electrophoresis (Agilent).

Circular RNA binding to TTR mRNA is evaluated by pull-down of circular RNA using a biotinylated oligo complementary to a specific sequence within the circle followed by RT-PCR. siRNA function is evaluated by measuring TTR target mRNA levels by RT-PCR in treated vs untreated cells. Expression of TTR protein is evaluated by western blotting.

### Example 24: Circular RNA with Modified Nucleotides was Generated and Selectively Bound Proteins

This Example demonstrates the generation of modified circular polyribonucleotide that supported protein binding. In addition, this Example demonstrates that circular RNA engineered with nucleotide modifications that selectively interacted with proteins involved in immune system monitoring had reduced immunogenicity as compared to unmodified RNA.

A non-naturally occurring circular RNA engineered to include complete or partial incorporation of modified nucleotides was produced. As shown in the following Example, full length modified linear RNA or a hybrid of modified and unmodified linear RNA was circularized and protein scaffolding was assessed through measurements of nLuc expression. In addition, selectively modified circular RNA had reduced interactions with proteins that activate immune related genes (q-PCR of MDA5, OAS and IFN-beta expression) in BJ cells, as compared to an unmodified circular RNA.

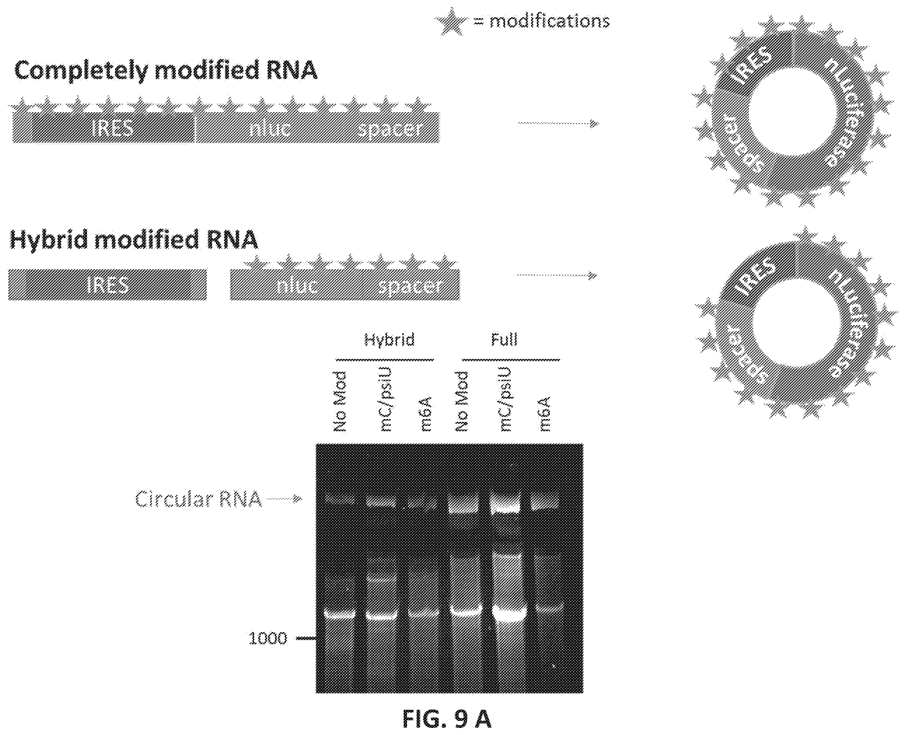

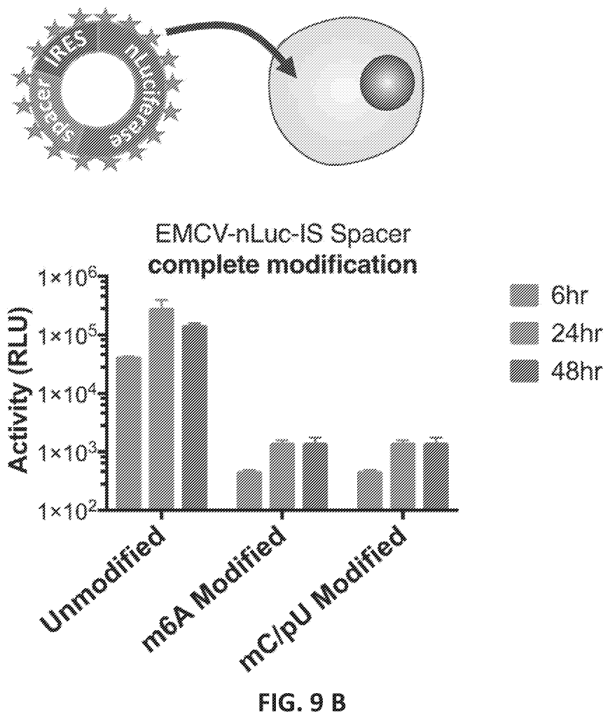

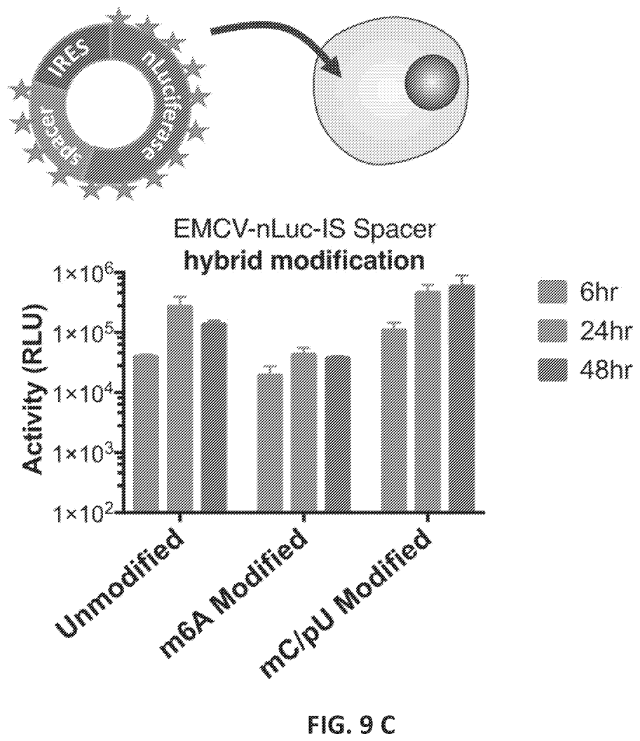

Circular RNA with a WT EMCV Nluc stop spacer was generated. For modification substitution, the modified nucleotides, pseudouridine and methylcytosine or m6A, were added in place of the standard unmodified nucleotides, uridine and cytosine or adenosine, respectively, during the in vitro transcription reaction. The WT EMCV IRES was synthesized separately from the nLuc ORF. The WT EMCV IRES was synthesized using either modified (completely modified) or unmodified nucleotides (hybrid modified). In contrast, the nLuc ORF sequence was synthesized using modified nucleotides, pseudouridine and methylcytosine or m6A, in place of the standard unmodified nucleotides, uridine and cytosine or adenosine, respectively, for the entire sequence during the in vitro transcription reaction. Following synthesis of the modified or unmodified IRES and the modified ORF, these two oligonucleotides were ligated together using T4 DNA ligase. As shown in FIG. 9A, completely modified (upper construct) or hybrid modified (lower construct) circular RNAs were generated.

To measure protein scaffolding efficiency, expression of nLuc from the completely modified or hybrid modified constructs was measured. After 0.1 pmol of linear and circular RNA was transfected into BJ fibroblasts for 6 h, nLuc expression was measured at 6 h, 24 h, 48 h and 72 h post-transfection.

As shown in FIG. 9B and FIG. 9C, completely modified circular RNA had greatly reduced protein binding capacity, as measured by protein translation output, as compared to unmodified circular RNA. In contrast, hybrid modification demonstrated as much as or increased binding to proteins, e.g., protein translation machinery.

To further measure protein scaffolding efficiency, completely modified circular RNA was transfected into cells and protein scaffolding to immune proteins was measured. The level of protein scaffolding to immune proteins that activate innate immune response genes was monitored in BJ cells transfected with unmodified circular RNA, or completely modified circular RNA with either pseudouridine and methylcytosine or m6A modifications. Total RNA was isolated from the cells using a phenol-based extraction reagent (Invitrogen) and subjected to reverse transcription to generate cDNA. qRT-PCR analysis for immune related genes was performed using a dye-based quantitative PCR mix (BioRad).

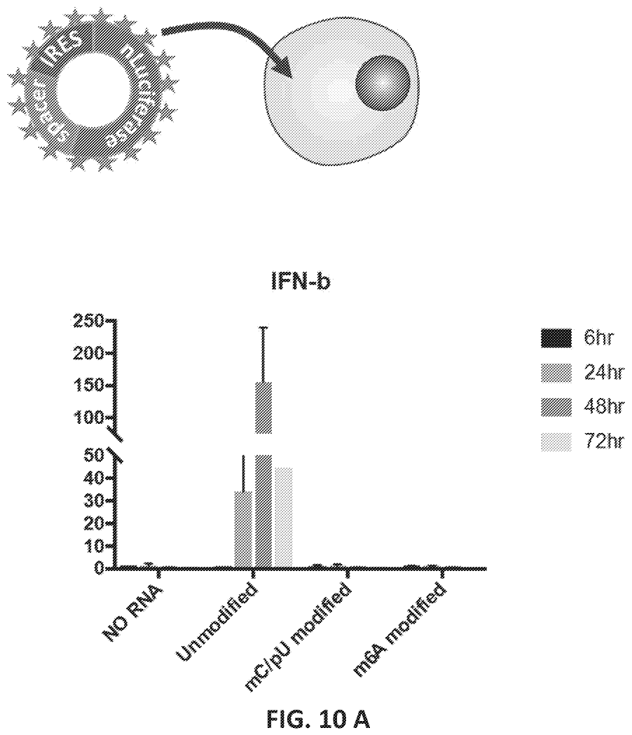

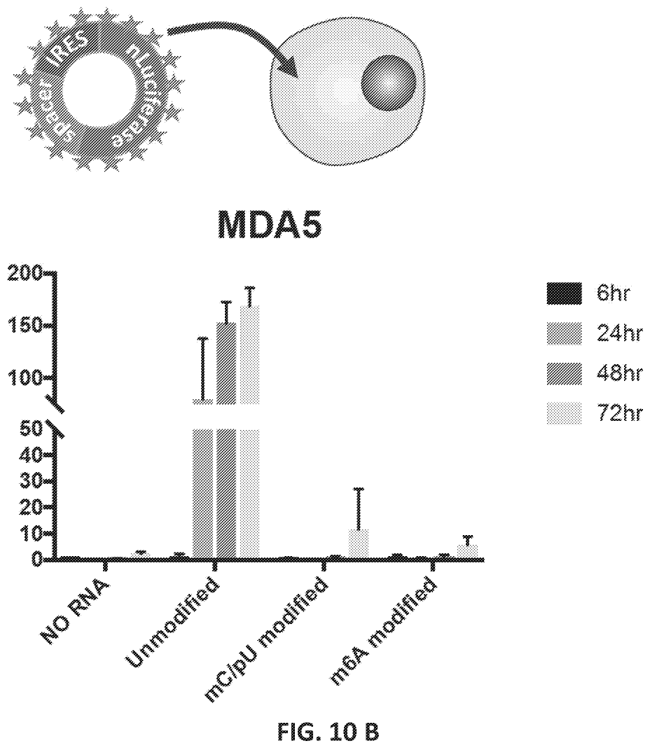

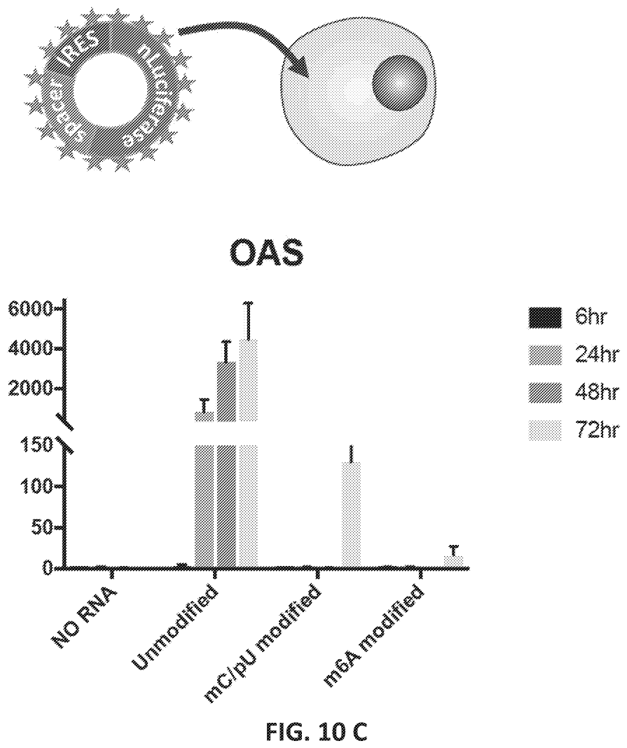

As shown in FIGS. 10A-C, qRT-PCR levels of immune related genes from BJ cells transfected with completely modified circular RNAs, both pseudouridine and methylcytosine or m6A completely modified circular RNAs, showed reduced levels of MDA5, OAS and IFN-beta expression as compared to unmodified circular RNA transfected cells, indicating reduced protein scaffolding between modified circular RNAs and immune proteins that activate immunogenic related genes. Thus, modification of circular RNA, as compared to unmodified circular RNA, had an impact on protein scaffolding. Selective modification allowed binding of protein translation machinery, while complete modification reduced binding to proteins that activate immunogenic related genes in transfected recipient cells.

### Example 25: Circular RNA with Modified Nucleotides Reduced Immunogenicity

This Example demonstrates the generation of modified circular polyribonucleotide that produced a protein product. In addition, this Example demonstrates circular RNA engineered with nucleotide modifications had reduced immunogenicity as compared to unmodified RNA.

A non-naturally occurring circular RNA engineered to include one or more desirable properties and with complete or partial incorporation of modified nucleotides was produced. As shown in the following Example, full length modified linear RNA or a hybrid of modified and unmodified linear RNA was circularized and expression of nLuc was assessed. In addition, modified circular RNA was shown to have reduced activation of immune related genes (q-PCR of MDA5, OAS and IFN-beta expression) in BJ cells, as compared to an unmodified circular RNA.

Circular RNA with a WT EMCV Nluc stop spacer was generated. For modification substitution, the modified nucleotides, pseudouridine and methylcytosine or m6A, were added in place of the standard unmodified nucleotides, uridine and cytosine or adenosine, respectively, during the in vitro transcription reaction. The WT EMCV IRES was synthesized separately from the nLuc ORF. The WT EMCV IRES was synthesized using either modified (completely modified) or unmodified nucleotides (hybrid modified). In contrast, the nLuc ORF sequence was synthesized using modified nucleotides, pseudouridine and methylcytosine or m6A, in place of the standard unmodified nucleotides, uridine and cytosine or adenosine, respectively, for the entire sequence during the in vitro transcription reaction. Following synthesis of the modified or unmodified IRES and the modified ORF, these two oligonucleotides were ligated together using T4 DNA ligase. As shown in FIG. 9, hybrid modified circular RNAs were generated.

To measure expression efficiency, hybrid modified circular RNA was transfected into cells and expression of immune proteins was measured. Expression levels of innate immune response genes were monitored in BJ cells transfected with unmodified circular RNA, or hybrid modified circular RNAs with either pseudouridine and methylcytosine or m6A modifications. Total RNA was isolated from the cells using a phenol-based extraction reagent (Invitrogen) and subjected to reverse transcription to generate cDNA. qRT-PCR analysis for immune related genes was performed using a dye-based quantitative PCR mix (BioRad).

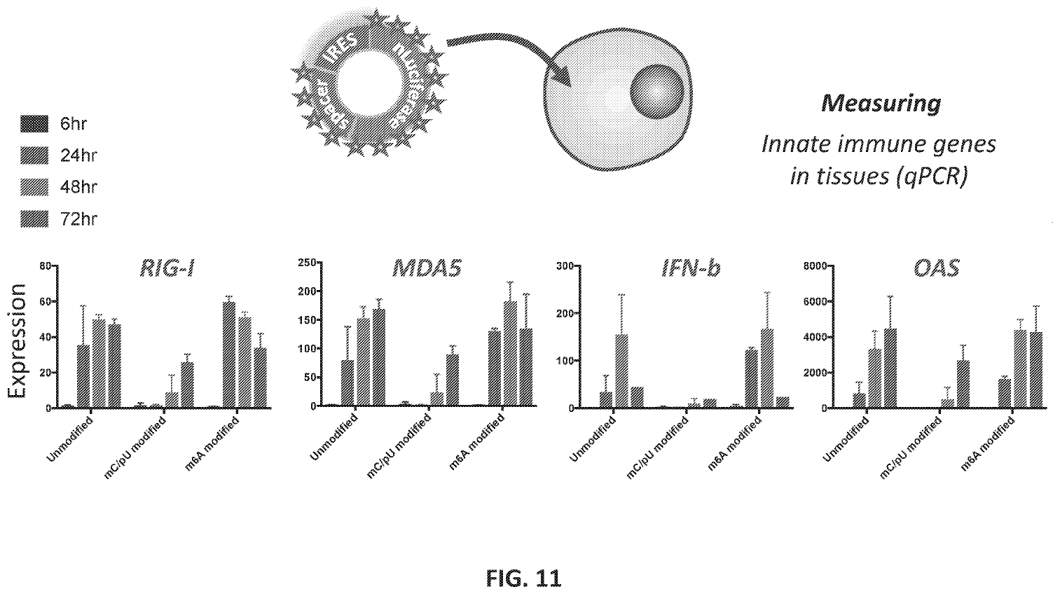

As shown in FIG. 11, qRT-PCR levels of immune related genes from BJ cells transfected with the hybrid modified circular RNAs, pseudouridine and methylcytosine hybrid modified circular RNAs showed reduced levels of RIG-I, MDA5, IFN-beta and OAS expression as compared to unmodified circular RNA transfected cells, indicating reduced immunogenicity of this hybrid modified circular RNA that activated the immunogenic related genes. Unlike the completely modified circular RNA shown in Example 24, m6A hybrid modified circular RNA showed similar levels of RIG-I, MDA5, IFN-beta and OAS expression as unmodified circular RNA transfected cells. Thus, modification of circular RNA, as compared to unmodified circular RNA, as well as the level of modification had an impact on activating immunogenic related genes.

### Example 26: Circular RNA Bound a Small Molecule

This Example demonstrates circular RNA binding a small molecule for sequestration/bio-activity.

Linear mango RNA aptamers fluoresce when bound by a small molecule, TO-1 biotin dye. As shown in the following Example, circular Mango RNA binds to the thiazol orange derivative, TO-1 biotin for sequestration/bio-activity.

Circular RNA was designed to include the mango RNA small molecule binding aptamer sites and a stabilizing stem: 5′-AATAGCCG GUCUACGGCC AUACCACCCU GAACGCGCCC GAUCUCGUCU GAUCUCGGAAGCUAAGCAGG GUCGGGCCUG GUUAGUACUU GGAUGGGAGA CCGCCUGGGAAUACCGGGUG CUGUAGGCGU CGACUUGCCA UGUGUAUGUG GGUACGAAGGAAGGAUUGGU AUGUGGUAUA UUCGUACCCA CAUACUCUGA UGAUCCUUCG GGAUCAUUCA UGGCAA CGGCTATT-3′ (SEQ ID NO: 18), as well as circularization sequences: 5′-AATAGCCG-3′ (SEQ ID NO: 19) and 5′-CGGCTATT-3′ (SEQ ID NO: 20).

Unmodified linear RNA was synthesized by in vitro transcription using T7 RNA polymerase from a DNA segment comprising the Mango RNA motif, stems and circularization sequences. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality is assessed by Urea-PAGE or through automated electrophoresis (Agilent).

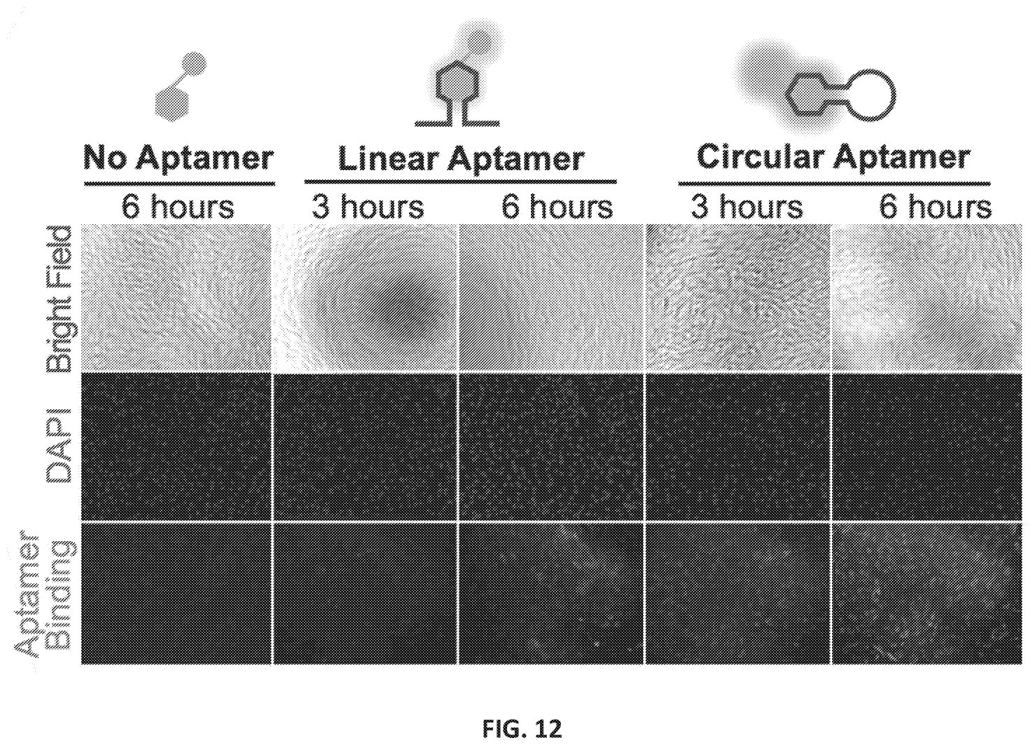

Circular RNA binding to TO-1 biotin was evaluated in vitro in BJ fibroblast cells, using fluorescent microscopy. When TO-1 biotin was bound to RNA it enhanced its fluorescence more than 100-fold. Linear or circular aptamers (50 nM) were added to the media of BJ fibroblast cultures, as well as a no-RNA control. A transfection reagent, lipofectamine, was added to ensure RNA delivery. Cultures were treated with TO-1 biotin and fluorescence was analyzed after 3 and 6 hours. As shown in FIG. 12, increased fluorescence/stability was detected from the circular aptamer, at both 3 and 6 hours.

More efficient delivery and more persistent fluorescence were observed with circular aptamers.

### Example 27: Circular RNA that Bound Protein

This Example demonstrates circular RNA binding to protein for sequestration.

Human antigen receptor (HuR) can be a pathogenic protein, e.g., it is known to bind and stabilize cancer related mRNA transcripts, such as mRNAs for proto-oncogenes, cytokines, growth factors, and invasion factors. HuR has a central tumorigenic activity by enabling multiple cancer phenotypes. Sequestration of HuR with circular RNA may attenuate tumorigenic growth in multiple cancers. As shown in the following Example, a circular RNA can bind to HuR for sequestration.

Circular RNA was designed to include the HuR RNA binding aptamer motifs: 5′-UCAUAAUCAA UUUAUUAUUUUCUUUUAUUUUA UUCACAUAAUUUUGUUUUU-3′ (SEQ ID NO: 21), 5′-AUUUUGUUUUUAA CAUUUC-3′(SEQ ID NO: 22), 5′-UCAUAAUCAAUUUAUUAUUUUCUUUUAUUUUAUUCACAUAAUUUUGUUU UUAUUUUGUUUUUAACAUUUC-3′(SEQ ID NO: 23) to competitively bind HuR and inhibit its binding/downstream functions.

Unmodified linear RNA was synthesized by in vitro transcription using T7 RNA polymerase from a DNA segment comprising the HuR RNA motif and protein binding sequence.

Transcribed RNA was purified with a Monarch RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

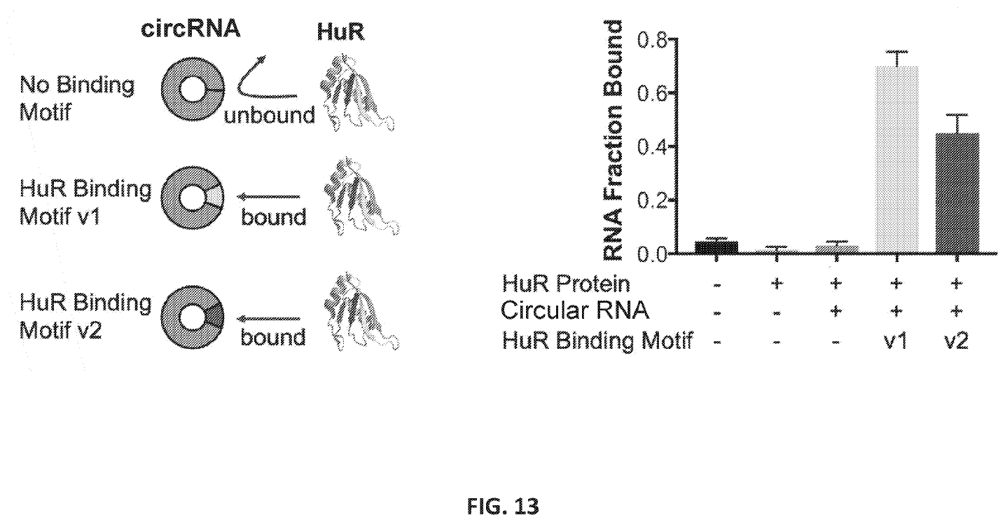

Circular RNA binding to HuR was evaluated in vitro by RNA immunoprecipitation (RIP) for HuR. Circular RNAs containing the HuR RNA-binding motif bound HuR protein, while circular RNAs lacking the HuR RNA-binding motif exhibited no binding above background (FIG. 13).

This result demonstrated selective binding of a circRNA to biomolecule of therapeutic interest.

### Example 28: Circular RNA with a Small Molecule Bound a Protein

This Example demonstrates circular RNA linked to a small molecule to bound and recruited a protein of choice.

Thalidomide, a clinically approved drug (Thalomid), is known to associate a member of the cells' protein degradation machinery, the E3 ubiquitin ligase. By conjugating thalidomide to circular RNA (e.g., via click chemistry), thalidomide-conjugated circular RNA can recruit cells' degradation machinery to a second, disease-causing protein (e.g., also targeted by the circular RNA). As shown in the following Example, a small molecule was conjugated to a circular RNA to bind E3 ubiquitin ligase Cereblon.

Circular RNA was designed to include reactive uridine residues (e.g., 5-azido-C3-UTP) for conjugation of alkyne-functionalized small molecules, known to interact with an intracellular protein of interest.

Linear RNA was synthesized by in vitro transcription using T7 RNA polymerase (Lucigen). All UTP was substituted with 5-azido-C3-UTP (Jena Biosciences) in the in vitro transcription reaction to generate azide-functionalized RNA. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Circular RNA was generated by splint ligation. RppH-treated linear RNA (100 uM) and splint DNA (200 uM) was annealed by heating at 75° C. for 5 min and gradual cooling at room temperature for 20 min. Ligation reaction was performed with T4 RNA ligase 2 (0.2 U/ul, New England Biolabs) for 4 hours at 37° C. The ligated mixture was purified by ethanol precipitation. To isolate circular RNA, the ligated mixture was separated on 4% denaturing UREA-PAGE. RNA on the gel was stained with SYBR-green (Thermo Fisher) and visualized with transilluminator (Transilluminators). Corresponding RNA bands for circular RNA were excised and crushed by gel breaker tubes (Ist Engineering). For elution of circular RNA, crushed gels with circular RNA were incubated with elution buffer (0.5M Sodium Acetate, 1 mM EDTA, 0.1% SDS) at 37° C. for an hour and supernatant was carefully harvested. The remaining crushed gel elution was subjected to another round of elution, and repeated total three times. Elution buffer with circular RNA was filtrated through a 0.45 um cellulose acetate filter to remove gel debris and circular RNA was purified/concentrated by ethanol precipitation.

Alkyne-functionalized thalidomide (Jena Bioscience) was conjugated to azide-functionalized circular RNA via Copper-catalyzed Azide-Alkyne click chemistry reactions (CuAAC) with the click chemistry reaction kit based on manufacturer's instructions (Jena Bioscience). Thalidomide-conjugated circular RNA was purified with an RNA clean up kit (New England Biolab).

Binding properties of the thalidomide-conjugated circular RNA were analyzed using GST pull-down followed by qPCR for RNA detection. For GST pull-down assay, thalidomide-conjugated circular RNA (2 nM) was incubated with GST-E3 ubiquitin ligase Cereblon (50 nM), which interacts with thalidomide, for 2 hours at room temperature in the presence of 25 mM Tris-Cl (pH7.0), 100 mM NaCl, 1 mM EDTA, 0.5% NP-40, 5% Glycerol. Azide-functionalized circular RNA without thalidomide conjugation was used as a negative control.

The RNA-protein mixture was further incubated for an hour at room temperature with GSH-agarose beads to assess GST-GSH interactions. After washing three times with binding buffer, the RNA specifically bound to the GSH-beads was extracted with Trizol (Thermo Fisher). The extracted circular RNA was reverse transcribed and detected by quantitative RT-PCR with primers specific for circular RNA (forward: TACGCCTGCAACTGTGTTGT (SEQ ID NO: 24), reverse: TCGATGATCTTGTCGTCGTC (SEQ ID NO: 25)).

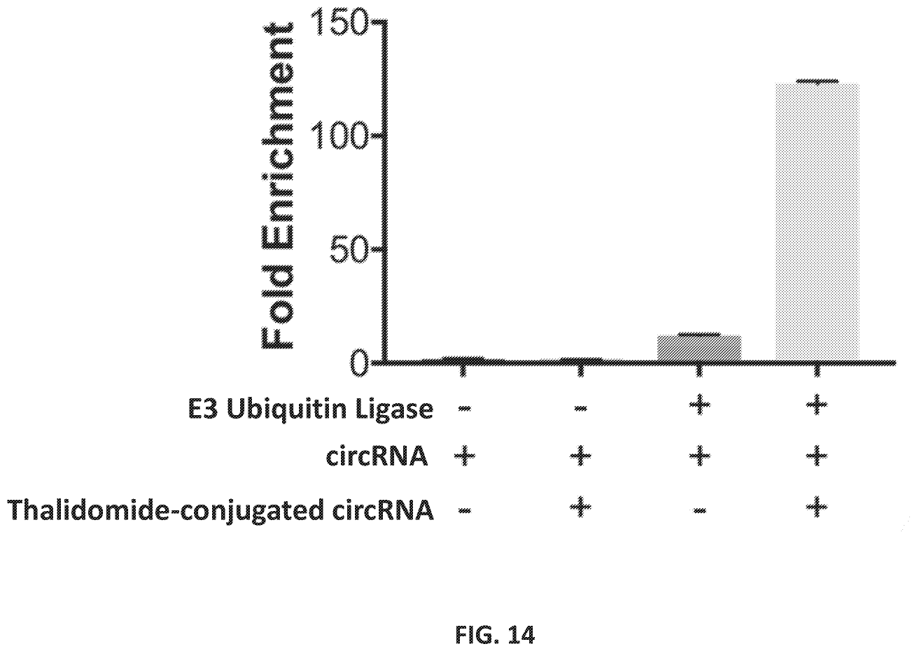

FIG. 14 demonstrates that circular RNA conjugated to the thalidomide small molecule was highly enriched in the GST pull-down assay, demonstrating that circular RNA with a small molecule, and bound to specific proteins through the small molecule.

### Example 29: Circular RNA Bound a Small Molecule

This Example demonstrates circular RNA linked to a small molecule specifically bound a secondary protein.

As shown in the following Example, a small molecule was clicked to a circular RNA to create a scaffold for specifically binding secondary proteins, e.g., E3 ubiquitin ligase and a target.

Circular RNA was designed to include reactive uridine residues (e.g., 5-azido-C3-UTP or 5-ethyl-UTP) for conjugation of alkyne-functionalized or azide-functionalized small molecules, for any downstream functionality.

Linear RNA was synthesized by in vitro transcription using T7 RNA polymerase (Lucigen). All UTP was substituted with 5-azido-C3-UTP or 5-ethyl UTP (Jena Biosciences) in the in vitro transcription reaction to generate azide-functionalized or alkyne functionalized RNA, respectively. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Circular RNA was generated by splint ligation. RppH-treated linear RNA (100 uM) and splint DNA (200 uM) was annealed by heating at 75° C. for 5 min and gradual cooling at room temperature for 20 min. Ligation reaction was performed with T4 RNA ligase 2 (0.2 U/ul, New England Biolabs) for 4 hours at 37° C. The ligated mixture was purified by ethanol precipitation.

To isolate circular RNA, the ligated mixture was separated on 6% denaturing UREA-PAGE. RNA on the gel was stained with SYBR-green (Thermo Fisher) and visualized with a transilluminator (Transilluminators). Corresponding RNA bands for circular RNA were excised and crushed by gel breaker tubes (1st Engineering). For elution of circular RNA, crushed gels with circular RNA were incubated with elution buffer (0.5M Sodium Acetate, 1 mM EDTA, 0.1% SDS) at 37° C. for an hour and supernatant was carefully harvested. The remaining crushed gel elution was subjected to another round of elution, and repeated for a total of three times. Elution buffer with circular RNA was filtrated through a 0.45 um cellulose acetate filter to remove gel debris and circular RNA was purified/concentrated by ethanol precipitation.

Alkyne-functionalized Alexa Fluor 488 dye or azide-functionalized Alexa Fluor 488 dye (Jena Bioscience) was conjugated to azide-functionalized circular RNA via Copper-catalyzed Azide-Alkyne click chemistry reactions (CuAAC) with the click chemistry reaction kit based on manufacturer's instructions (Jena Bioscience). Alexa Fluor 488 dye-conjugated circular RNA was purified with an RNA clean up kit (New England Biolab).

The dye conjugation was monitored by separating circular RNA on 6% denaturing UREA-PAGE. Alexa Fluore dye-unconjugated and -conjugated circular RNA were separated on the gel in parallel for comparison. Fluorescence from the RNA on the gel was monitored by iBright Imaging System (Invitrogen). After monitoring fluorescence, the gel was stained with SYBR safe and RNA on the gel was visualized by iBright Imaging System (Invitrogen).

Circular RNA containing a small molecule Alexa Fluor 488 was shown to fluoresce demonstrating that circular RNA can contain a functional small molecule.

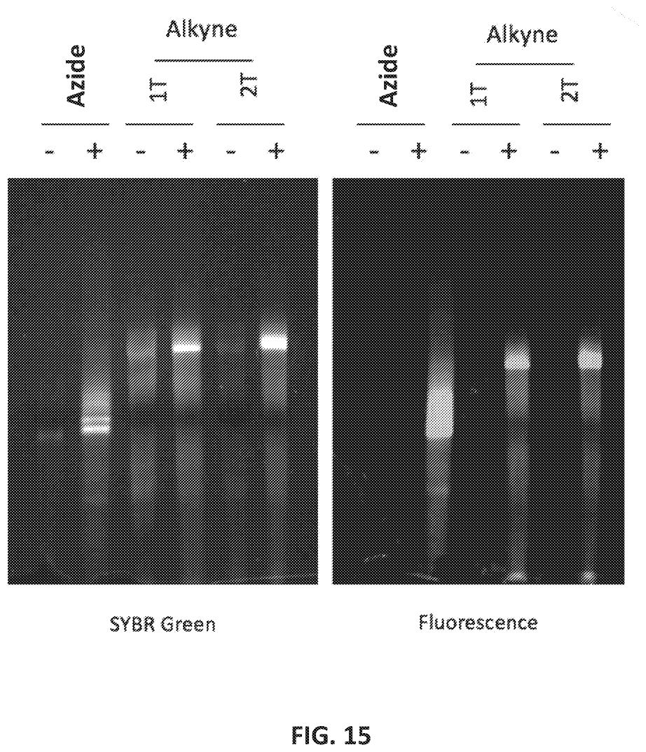

As illustrated in FIG. 15, circular RNA conjugated to the thalidomide small molecule produced a descrete PCR product as detected by fluorescence, demonstrating that circular RNA conjugated to a small molecule specifically interacted with a secondary protein.

### Example 30: Circular RNA that Binds Two Different Small Molecules

This Example describes two different proteins of choice thare are recruited by a circular RNA that is linked to small molecules.

Thalidomide, a clinically approved drug (Thalomid), is known to associate with a member of the cells' protein degradation machinery, the E3 ubiquitin ligase cereblon. By conjugating thalidomide to circular RNA (e.g., via click chemistry), thalidomide-conjugated circular RNA can recruit cells' degradation machinery to a second, disease-causing protein (e.g., also targeted by the circular RNA). As shown in the following Example, two small molecules (thalidomide and JQ1) are conjugated to a circular RNA to bind (1) E3 ubiquitin ligase Cereblon for ubiquitination and subsequent degradation of a neighboring protein and (2) BET family proteins through JQ1, which is a small molecule inhibitor that binds to BET family proteins.

Circular RNA is designed to include reactive uridine residues (e.g., 5-azido-C3-UTP) for conjugation of alkyne-functionalized small molecules, known to interact with an intracellular protein of interest.

Linear RNA is synthesized by in vitro transcription using T7 RNA polymerase (Lucigen). All UTP is substituted with 5-azido-C3-UTP (Jena Biosciences) in the in vitro transcription reaction to generate azide-functionalized RNA. Synthesized linear RNA is purified with an RNA clean up kit (New England Biolabs) and is subjected to RNA 5′ Pyrophosphohydrolase (RppH, New England Biolabs) treatment to remove pyrophosphate. RppH-treated linear RNA is purified with an RNA clean up kit (New England Biolabs).

Circular RNA is generated by splint ligation. RppH-treated linear RNA (100 uM) and splint DNA (200 uM) is annealed by heating at 75° C. for 5 min and is gradually cooled at room temperature for 20 min. Ligation reaction is performed with T4 RNA ligase 2 (0.2 U/ul, New England Biolabs) for 4 hours at 37° C. The ligated mixture is purified by ethanol precipitation. To isolate circular RNA, the ligated mixture is separated on 4% denaturing UREA-PAGE. RNA on the gel is stained with SYBR-green (Thermo Fisher) and is visualized with transilluminator (Transilluminators). Corresponding RNA bands for circular RNA are excised and crushed by gel breaker tubes (1st Engineering). For elution of circular RNA, crushed gels with circular RNA are incubated with elution buffer (0.5M Sodium Acetate, 1 mM EDTA, 0.1% SDS) at 37° C. for an hour and supernatant is carefully harvested. The remaining crushed gel elution is subjected to another round of elution, and is repeated total three times. Elution buffer with circular RNA is filtrated through a 0.45 um cellulose acetate filter to remove gel debris and circular RNA is purified/concentrated by ethanol precipitation.

Alkyne-functionalized thalidomide and alkyne-functionalized JQ1 (Jena Bioscience) are conjugated to azide-functionalized circular RNA via Copper-catalyzed Azide-Alkyne click chemistry reactions (CuAAC) with the click chemistry reaction kit based on manufacturer's instructions (Jena Bioscience). For comparison, three different kinds of small molecule conjugated circular RNA are generated: RNA with both JQ1 and thalidomide, thalidomide only, and JQ1 only. Small molecule-conjugated circular RNA are purified with an RNA clean up kit (New England Biolab).

Small molecule-conjugated circular RNA binding to E3 ubiquitin ligase CRBN and BET family proteins are analyzed using GST pull-down. GST-CRBN (Abcam) and one of the BET family protein, Bromodomain containing protein 4 (BRD4, BPSBiosciences) are used for this experiement. For GST pull-down assay, thalidomide and JQ1 conjugated-circular RNA (2 nM) are incubated with GST-CRBN and BRD4 (50 nM each) for 2 hours at room temperature in the presence of 25 mM Tris-Cl (pH7.0), 100 mM NaCl, 1 mM EDTA, 0.5% NP-40, 5% Glycerol. Azide-functionalized circular RNA without conjugation, thalidomide conjugated RNA, and JQ1 conjugated RNA are used as negative controls. RNA-protein mixture is further incubated with GSH-agarose bead to allow GST-GSH interaction for an hour at room temperature. After washing three times with binding buffer, the bead is separated to two equal parts. To monitor protein binding, one part of the bead is boiled in the presence of Lammli Sample Buffer (Bio-Rad) and is subjected to western blot with BRD4 antibody (for detecting BRD4 protein) and GST antibody (for detecting GST-CRBN). To monitor RNA recruitment, the RNA on the bead is extracted with Trizol (Thermo Fisher) and the extracted circular RNA is reverse transcribed and is detected by quantitative RT-PCR with primers specific for circular form of RNA

(forward:
TACGCCTGCAACTGTGTTGT (SEQ ID NO: 24),
reverse:
TCGATGATCTTGTCGTCGTC (SEQ ID NO: 25)).

It is expected that circular RNA containing the thalidomide and JQ1 small molecules is highly enriched in the GST pull down for both CRBN as well as BET domain protein BRD4, demonstrating that not only can circular RNA contain a small molecule, but it can bind to two specific proteins using this small molecule conjugate to degrade the protein of choice.

### Example 31: Circular RNA that Binds Carbohydrates

This Example describes circular RNA binding to carbohydrates.

Sialyl Lewis X is a tetrasaccharide glycoconjugate of membrane proteins. It acts as a ligand for selectin proteins during cell adhesion. As shown in the following Example, the circular RNA binds to Sialyl Lewis X to inhibit cell adhesion.

An engineered circular RNA is designed to include a Sialyl Lewis X binding sequence

(e.g., 5′-
CCGUAAUACGACUCACUAUAGGGGAGCUCGGUACCGAAUUCAAGGUACUCU
GUGCUUGUCGAUGUGUAUUGAUGGCACUUUCGAGUCAACGAGUUGACAGAA
CAAGUAGUCAAGCUUUGCAGAGAGGAUCCUU-3′
(SEQ ID NO: 26)).

Unmodified linear RNA is synthesized by in vitro transcription using T7 RNA polymerase from a DNA segment comprising Sialyl Lewis X binding sequence. Transcribed RNA is purified with an RNA purification system (QIAGEN), treated with alkaline phosphatase (ThermoFisher Scientific, EF0652) following the manufacturer's instructions, and purified again with the RNA purification system. Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to Sialyl Lewis X is to measure Sialyl Lews X-mediated cell adhesion. E-selectin recognizes Sialyl Lews X, and the surface of promyelocytic leukemia cell line HL60 is rich in Sialyl Lews X, especially after TNF-α treatment. Recombinant soluble E-selectin (Calbiochem) is added to the microtiter plate (250 ng/well) in 0.05 M NaHCO₃ at pH 9.2 (10 μg/ml) and is incubated overnight at 4° C. Circular RNA (10 μg/mL) with or without the Sialyl Lewis X binding site is then incubated. TNF-α activated (10 ng/ml for 20 h) HL60 human promyelocytic leukemia cells are incubated for 30 min at room temperature on the plate, are washed, and the numbers of adhered cells are measured.

### Example 32: Circular RNA that Binds Virus

This Example describes circular RNA binding to virus.

The influenza virus has two membrane glycoprotein components including hemagglutinin (HA) and neuraminidase (NA). About 900 and 300 copies of HA and NA, respectively, are expressed on the surface of each viral particle. As shown in the following Example, an engineered circular RNA is designed to bind to hemagglutinin for viral binding.

Circular RNA is designed to include a Hemagglutinin binding site (e.g., 5′-GGGAGAAUUCCGACCAGAAGGGUUAGCAGUCGGCAUGCGGUACAGACAGACCUU UCCUCUCUCCUUCCUCUUCU-3′ (SEQ ID NO: 27)) to bind to the surface of the influenza virus.

Unmodified linear RNA is synthesized by in vitro transcription using T7 RNA polymerase from a DNA segment comprising hemagglutinin binding sequence. Transcribed RNA is purified with an RNA purification system (QIAGEN), treated with alkaline phosphatase (ThermoFisher Scientific, EF0652) following the manufacturer's instructions, and purified again with the RNA purification system. Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

One method to assess circular RNA binding to hemagglutinin is inhibitory effects of RNA aptamers on HA-induced membrane fusion. When hemagglutinin is bound to circular RNA, membrane fusion occurs less frequently than that of unbound circular RNA.

HA-induced membrane fusion is examined by using fluorescently labelled virus and human red blood cell (RBC) ghost membranes. The viral membrane of A/Panama/2007/1999 (H3N2) is labelled with a fluorescent lipid probe, octadecyl rhodamine B (R18; Molecular Probes).

For the fusion-inhibition assay, the H3N2 virus (0.05-0.1 mg total protein/ml) mixed with a circular RNA (0.5 or 5 mM) is added to ghost membranes on coverslips mounted in a metal chamber. Upon viral fusion with ghost membranes, lipid intermixing between the viral and ghost membranes induces fluorescence dequenching of R18.

### Example 33: Circular RNA that Binds Cells

This Example describes circular RNA binding to target cell types.

In this Example, an engineered circular RNA is designed through one of the methods described previously. Circular RNA and linear RNA are designed to include a mango aptamer, a stabilizing stem, and a non-coding region: a transferrin aptamer (e.g., GGGGGAUCAAUCCAAGGGACCCGGAAACGCUCCCUUACACCCC (SEQ ID NO: 28)).

This aptamer region binds the transferrin receptor allowing the RNA to bind to cells that express the receptor. Transferrin receptor is expressed on a variety of cell-types, including red blood cells and some cancer cells. As a negative control, RNA is designed to not include the aptamer region.

HeLa cells are cervical cancer cells that are known to express the transferrin receptor. HeLa cells are grown under standard conditions (in DMEM, with 10% FBS at 37° C. under 5% CO2). Cells are passaged regularly to maintain exponential growth. Circular RNA binding to TO-1 biotin is evaluated in vitro in HeLa cells, using fluorescent microscopy. When TO-1 biotin is bound to RNA it enhances its fluorescence more than 100-fold. Circular RNA with or without aptamers (50 nM) is added to the media of HeLa cultures, as well as a no-RNA control. A lipid-based transfection reagent (Thermo Fisher Scientific) is added to ensure RNA delivery. Cultures are treated with TO-1 biotin and fluorescence is analyzed after 3 and 6 hours.

### Example 34: Circular RNA that Binds Aptamer

This Example describes circular RNA binding to an aptamer.

An engineered circular RNA is designed to include one or more novel binding sequences for RNA aptamers. RNA aptamers are targeted for circular RNA binding through complementarity. As shown in the following Example, the circular RNA binds complementary to the LIN28A binding aptamer for sequestration.

Circular RNA is designed to include the complementary sequence to the LIN28A binding aptamer sequence, 5′-GGGGUAGUGAUUUUACCCUGGAGAU-3′(SEQ ID NO: 12).

Unmodified linear RNA is synthesized by in vitro transcription using T7 RNA polymerase from a DNA segment having the complementary LIN28A binding aptamer sequence. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M) or T4 RNA ligase 2 (New England Bio, Inc., M0239S) and the circular RNA is isolated following enrichment with RNase R treatment. RNA quality is assessed by agarose gel or through automated electrophoresis (Agilent).

Circular RNA binding to the LIN28A binding aptamer is evaluated by an oligonucleotide pull-down-qPCR assay, in which modified oligonucleotides complementary to the circular RNA are used to pull-down the LIN28A binding aptamer, which is reverse-transcribed and qPCR amplified.

### Example 35: Circular RNA Bound a Transcription Factor

This Example demonstrates circular RNA bound to protein for sequestration. NF-kB is a family of transcription factors that activate transcription and induce survival pathways. As shown in the following Example, the circular RNA bound to NF-kB for sequestration.

Circular RNA was designed to include the NF-kB RNA binding aptamer motifs: 5′-aaaaaaaaaaGATCTTGAAACTGTTTTAAGGTTGGCCGATCTTaaaaaa-3′(SEQ ID NO: 29) to competitively bind NF-kB and inhibit its binding/downstream functions. Poly(A) stretches were added to the internal binding motif to (1) make the RNA oligo amenable to ligation and to maintain the secondary structure of the aptamer. Correct folding was checked using RNAfold Web Server. As a control, a scrambled RNA sequence was used (aaaaaaaTTCTCCGAACGTGTCACGTTTCAAGAGAACGTGACACGTTCGGAGAAaaaaaa(SEQ ID NO: 30). This scrambled RNA sequence folds into a 3D structure similar to the aptamer, but does not target any proteins, as described in Mi et al., Mol Ther. 2008 Jan. 16(1):66-73.

RNA with the NF-kB binding aptamer motif was synthesized by a commercial vendor (IDT) with a 5′ monophosphate group and a 3′ hydroxyl group. RNA ligase 1 (New England Biolabs, M0204S) was used to ligate the RNA oligo. RNase R was used to remove residual linear RNA from the samples, according to manufacturer's instructions (Lucigen, RNR07250). Additionally, circular mRNA was purified by extracting the circular RNA from a 15% Urea PAGE gel. Circular RNA was eluted from the gel in a buffer containing: 0.5M Sodium Acetate, 0.1% SDS, 1 mM EDTA. Residual gel debris or salts from the gel extraction were removed by running the elution through a spin column (New England Biolabs, T2030S). RNA was eluted in toRNA storage buffer (1 mM sodium citrate, Thermo Fisher, AM7000) and RNA integrity was assessed by Urea-PAGE or through automated gel capillary electrophoresis (Agilent).

Electrophoretic mobility shift assay (EMSA) was performed to assess circular RNA binding affinity to NF-kB. One pmole of linear or circular RNA was incubated with recombinant NF-kB p50 subunit (Caymen Chemical, 10009818) at varying concentrations over the RNA concentration (i.e., 0, 0.1, 1, 10 pmoles of protein) for 20 minutes at room temperature in a buffered reaction (20 mM Tris-HCl, pH 8.0, 50 mM NaCl, 1 mM MgCl2). Samples were run a 6% TBE Urea gel for 25 minutes at 200V. Gels were stained with SybrGold (Thermo Scientific, S11494) and imaged with a blue E-gel imaging system (Thermo Scientific, 4466612).

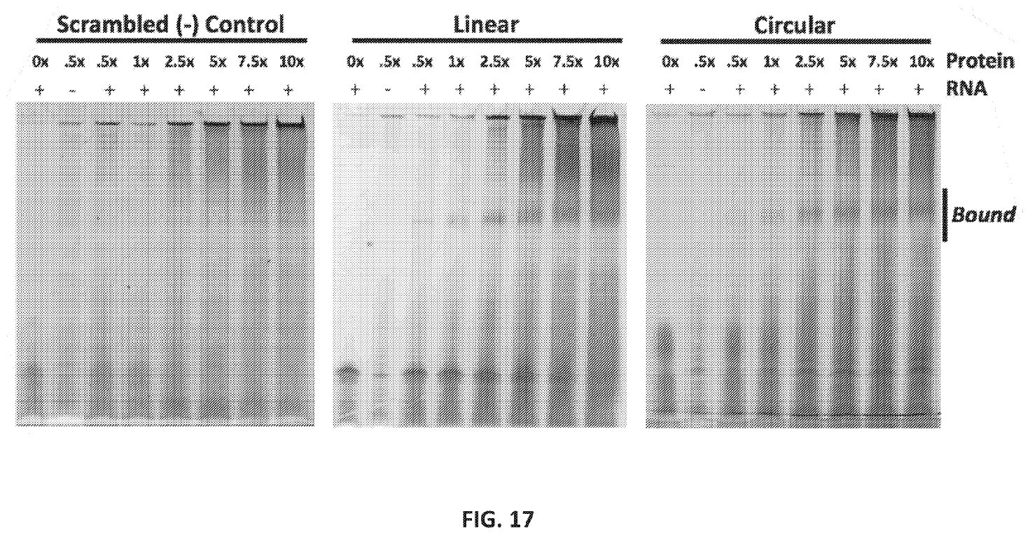

As demonstrated in FIG. 17, RNA with scrambled binding aptamer sequences did not show binding affinity to the p50 subunit of NF-kB. Both linear and circular versions of the NF-kB binding aptamer sequence bound to the p50 subunit with similar affinities.

Circular RNA binding to NF-kB was evaluated in vitro by EMSA for NF-kB. NF-kB selectively bound circular RNAs containing the NF-kB RNA binding aptamer motif. This result demonstrated that biomolecules of interests were selectively bound by sequences in circular RNA.

### Example 36: Circular RNA Sequestered Target Protein and Inhibited Function

This Example demonstrates circular RNA binds to protein in cells and this sequestration leads to inhibition of function. As shown in the following Example, the circular RNA binds to NF-kB for sequestration leading to inhibition of survival activated by NF-kB in cells.

Circular, linear, and linear scrambled RNA were designed and synthesized as previously described.

NF-kB function in non-small cell lung cancer (NSCLC) cell line, A549s, after delivery of a circular RNA with a NF-kB binding aptamer sequence was determined by measuring cell viability by MTT Assay (Thermo Scientific, V13154). In short, A549 cells were transfected with 1 pmole of linear, linear scrambled, or circular RNA after complexation with lipid transfection reagent (Thermo Scientific, LMRNA003). Viability was measured by MTT assay performed according to the manufacturer's instructions

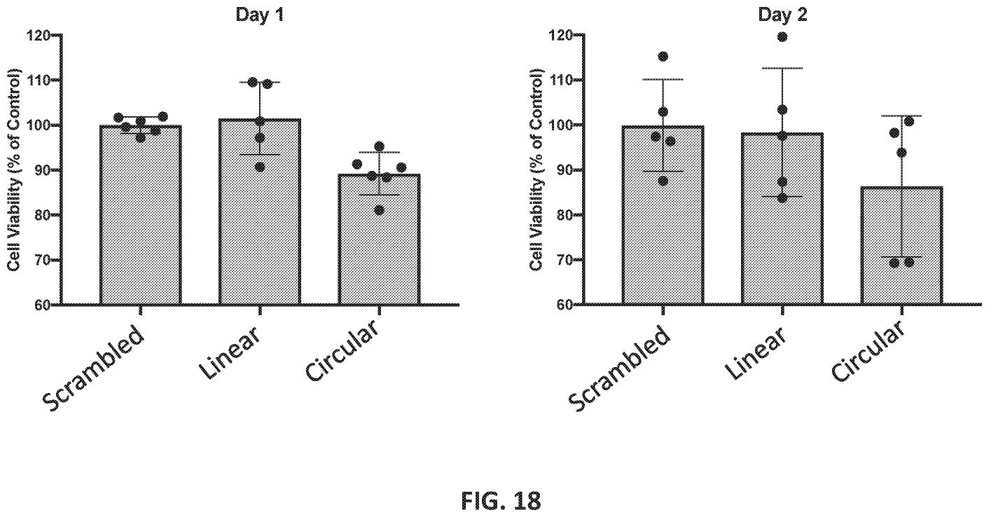

As demonstrated in FIG. 18, cells treated with linear RNA demonstrated no change in viability at day 1 and a slight decrease in viability at day 2 (101% viability on Day 1, and 97% on Day 2). In contrast, cells treated with the circular RNA demonstrated a measurable decrease in viability at day 1 and greater increase by day 2 (89% on Day 1 and 86% on Day 2).

Overall, the results demonstrated that circular RNA bound NF-kB in cells and inhibited NF-kB activation of survival pathways.

### Example 37: Circular RNA Bound and Sequestered Protein to Affect Chemotherapeutic Sensitization

This Example demonstrates circular RNA binds to a target protein in cells leading to the inhibition of the target protein's signaling pathways. As shown in the following Example, the circular RNA sequestered NF-kB in chemoresistant cells and inhibited NF-kB's signaling thereby re-sensitizing the cells to the chemotherapeutic.

Linear, linear scrambled, and circular RNA were designed and synthesized as previously described.

The effect of NF-kB sequestration in chemoresistant non-small cell lung cancer (NSCLC) cell line, A549s, was determined after delivery of a circular RNA targeting NF-kB and exposure to the chemotherapeutic agent. Cell viability was determined by MTT Assay (Thermo Scientific, V13154). In short, A549 cells were transfected with 1 pmole of a scrambled linear control, linear, or circular RNA after complexation with lipid transfection reagent (Thermo Scientific, LMRNA003). 24 hours post-transfection cells were treated with 5 uM doxorubicin for an additional 18 hours. Viability was measured by MTT assay performed according to the manufacturer's instructions. Doxorubicin treatment was repeated at 48- and 72-hours post transfection.

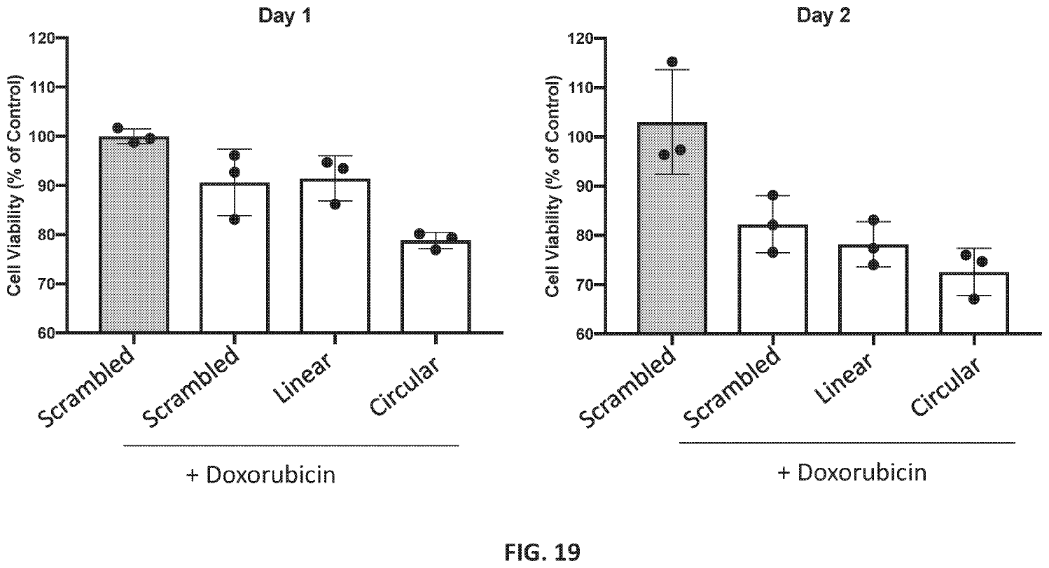

As demonstrated in FIG. 19, doxorubicin treatment with scrambled linear RNA (control) did not affect cell viability in the dox-resistant A549 lung cancer cell line at day 1. Co-treatment of doxorubicin with linear RNA decreased cell viability at day 2 (78% survival). In contrast, co-treatment with the circular aptamer resulted in more cell death at both days 1 and 2 (79% survival at day 1 and 73% survival at day 2).

Overall, the results demonstrated that circular RNA bound NF-kB in cells and inhibited NF-kB survival signaling, thereby increasing sensitivity of the cells to the chemotherapeutic, doxorubicin.

### Example 38: Circular RNA Tagged the Target Protein for Degradation

This Example demonstrates circular RNA linked to small molecules recruited two different proteins of choice and thereby tagged the target protein for degradation.

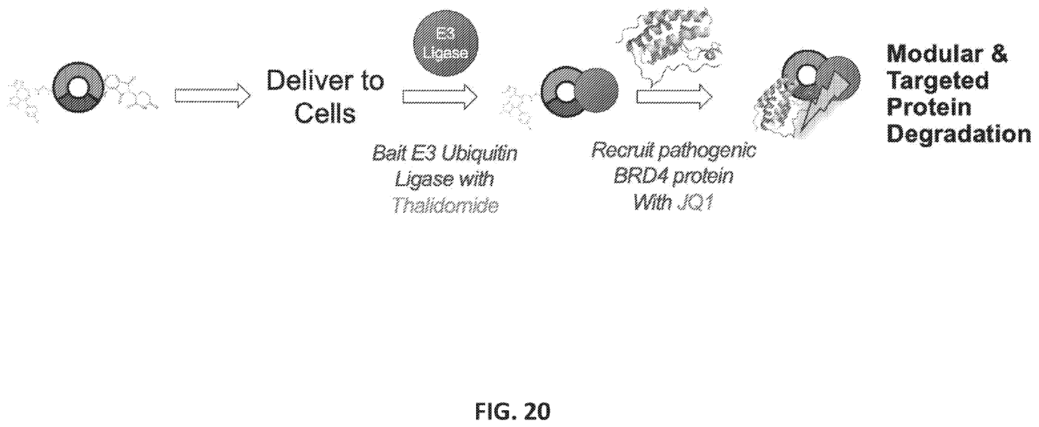

Thalidomide, a clinically approved drug (Revlimid), is known to associate with a member of the cells' protein degradation machinery, the E3 ubiquitin ligase. By conjugating thalidomide to circular RNA (e.g., via click chemistry), thalidomide-conjugated circular RNA can recruit cells' degradation machinery to a second, disease-causing protein (e.g., also targeted by the circular RNA). FIG. 20 is a schematic showing an exemplary circular RNA that is delivered into cells and tags a target BRD4 protein in the cells for degradation by ubiquitin system. As shown in the following Example, two small molecules (thalidomide and JQ1) were conjugated to a circular RNA to bind (1) E3 ubiquitin ligase Cereblon for ubiquitination and subsequent degradation of a neighboring protein; and (2) BET family proteins through JQ1 that is small molecule inhibitor that binds BET family proteins.

Circular RNA was designed to include multiple (49 residues) reactive uridine residues (e.g., 5-azido-C3-UTP) for conjugation of alkyne-functionalized small molecules, known to interact with an intracellular protein of interest.

Linear RNA was synthesized by in vitro transcription using T7 RNA polymerase (Lucigen). All UTP was substituted with 5-azido-C3-UTP (Jena Biosciences) in the in vitro transcription reaction to generate azide-functionalized RNA. Synthesized linear RNA was purified with an RNA clean up kit (New England Biolabs) and subjected to RNA 5′ Pyrophosphohydrolase (RppH, New England Biolabs) treatment to remove pyrophosphate. RppH-treated linear RNA was purified with an RNA clean up kit (New England Biolabs).

Circular RNA was generated by splint ligation. RppH-treated linear RNA (100 uM) and splint DNA (200 uM) was annealed by heating at 75° C. for 5 min and gradual cooling at room temperature for 20 min. Ligation reaction was performed with T4 RNA ligase 2 (0.2 U/ul, New England Biolabs) for 4 hours at 37° C. The ligated mixture was purified by ethanol precipitation. To isolate circular RNA, the ligated mixture was separated on 4% denaturing UREA-PAGE. RNA on the gel was stained with SYBR-green (Thermo Fisher) and visualized with transilluminator (Transilluminators). Corresponding RNA bands for circular RNA were excised and crushed by gel breaker tubes (Ist Engineering). For elution of circular RNA, crushed gels with circular RNA were incubated with elution buffer (0.5M Sodium Acetate, 1 mM EDTA, 0.1% SDS) at 37° C. for an hour and supernatant was carefully harvested. The remaining crushed gel was subjected to another round of elution, and repeated a total of three times. Elution buffer with circular RNA was filtrated through a 0.45 μm cellulose acetate filter to remove gel debris and circular RNA was purified/concentrated by ethanol precipitation.

Alkyne-functionalized thalidomide and/or JQ1 (thienotriazolodiazepine, Jena Bioscience) was conjugated to azide-functionalized circular RNA via Copper-catalyzed Azide-Alkyne click chemistry reactions (CuAAC) with the click chemistry reaction kit based on manufacturer's instructions (Jena Bioscience). For comparison, three different kinds of small molecules were conjugated to circular RNA; RNA with both JQ1 and thalidomide, thalidomide only, or JQ1 only. Small molecule-conjugated circular RNA was purified with an RNA clean up kit (New England Biolab).

These different RNAs were then transfected into HEK293T cells to monitor degradation of target protein using by lipid transfection reagent (Invitrogen) according to the manufacturer's instruction. 1 pmole of each RNA was used to transfect HEK293T cells and the cells were plated into 12 well plates (2 nM final). In the case of circular RNA conjugated with both JQ1 and thalidomide, 3 pmole of RNA was transfected into HEK293T cells to test the effect of different concentrations of circular RNA on BRD4 degradation (6 nM final). As a positive control, PROTAC dBET1 (Tocris Biosciences) that has both JQ1 and thalidomide, and is known to degrade BRD4 protein in cells through CRBN recruitment, was used (2 uM, 10 uM concentration). For a negarive control, carrier only and circular RNA without conjugation were used. After 24 hours transfection, cells were harvested by adding RIPA buffer directly onto the plate.

Small molecule-conjugated circular RNA binding to E3 ubiquitin ligase CRBN and BET famiy proteins degrading ability was analyzed using western blot. Briefly, 12 ug of protein was resolved on 4%-12% gradient Bis-Tris gel (Thermo Fisher Scientific) and transferred to nitrocelluose membrane using a blot transfer system (Thermo Fisher Scientific). Rabbit anti-BRD4 antibody (Abcam) was used to detect BRD4 protein and rabbit anti-alpha tubulin antibody (Abcam) was used to detect alpha tubulin as a loading control. The chemiluminoscence signal from protein bands of BRD4 and alpha tubulin were monitored by an Fc imaging system (LI-COR).

BRD4 protein levels as well as alpha tubulin as a loading control were also measured using densitometry using ImageJ.

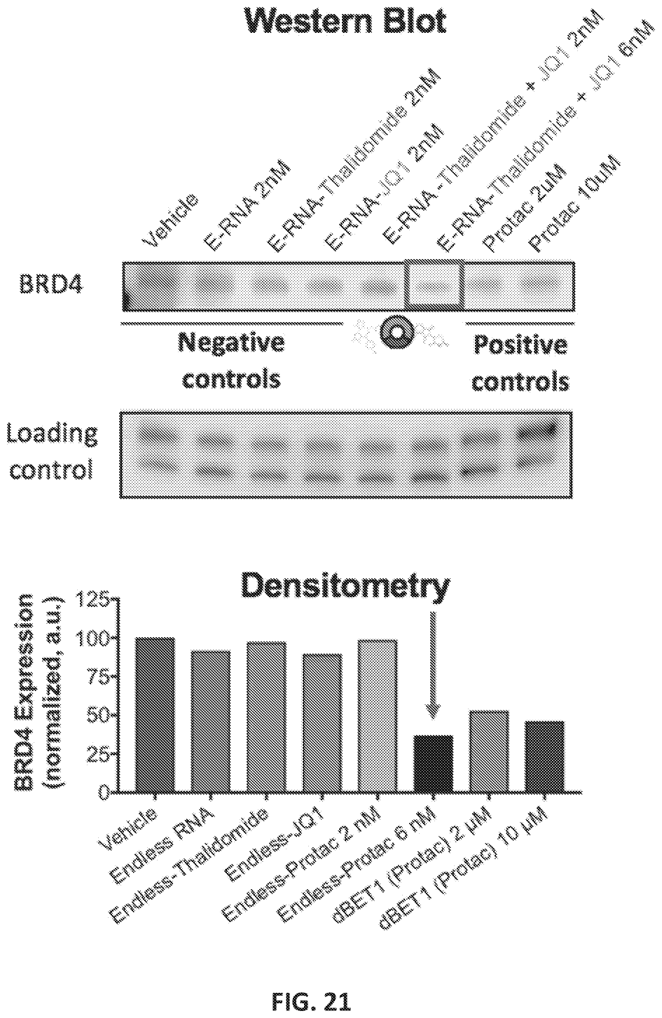

As shown in FIG. 21, circular RNA containing the thalidomide and JQ1 small molecules was able to degrade BRD4, as demonstrated by the normalized levels of BRD4. This result demonstrated that circular RNA with a small molecule bound to two specific proteins using the small molecule conjugate to degrade the target protein.

### Example 39: Circular RNA Bound a Small Molecule Longer than its Linear Counterpart

This Example demonstrates circular RNA binding a small molecule for sequestration/bio-activity. As shown in the following Example, the circular RNA is more stable than its linear counterpart.

Linear mango RNA aptamers fluoresce when bound by a small molecule, TO-1 biotin dye. As shown in the following Example, circular Mango RNA bound to the thiazol orange derivative, TO-1 biotin for sequestration/bio-activity.

Circular RNA was designed to include the mango RNA small molecule binding sites and a stabilizing stem: 5′-AATAGCCG GUCUACGGCC AUACCACCCU GAACGCGCCC GAUCUCGUCU GAUCUCGGAAGCUAAGCAGG GUCGGGCCUG GUUAGUACUU GGAUGGGAGA CCGCCUGGGAAUACCGGGUG CUGUAGGCGU CGACUUGCCA UGUGUAUGUG GGUACGAAGGAAGGAUUGGU AUGUGGUAUA UUCGUACCCA CAUACUCUGA UGAUCCUUCG GGAUCAUUCA UGGCAA CGGCTATT-3′(SEQ ID NO: 18), as well as circularization sequences: 5′-AATAGCCG-3′ (SEQ ID NO: 19) and 5′-CGGCTATT-3′ (SEQ ID NO: 20).

Unmodified linear RNA was synthesized by in vitro transcription using T7 RNA polymerase from a DNA segment comprising the Mango RNA motif, stems and circularization sequences. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

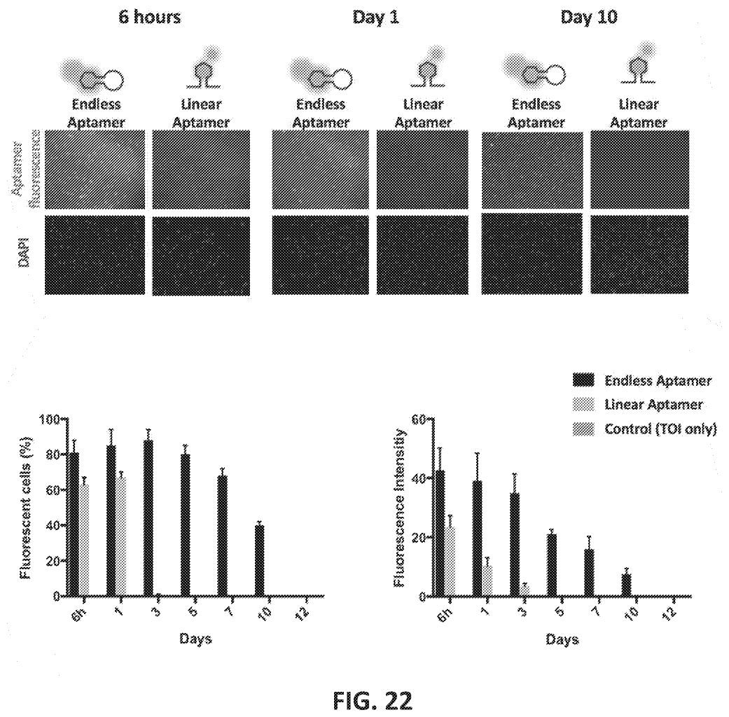

Circular RNA binding to TO-1 biotin was evaluated in vitro in HeLa cells, using fluorescent microscopy. When TO-1 biotin was bound to RNA it enhanced its fluorescence more than 100-fold. Linear or circular aptamers (50 nM) were added to the media of BJ fibroblast cultures, as well as a no-RNA control. A transfection reagent, lipofectamine, was added to ensure RNA delivery. Cultures were treated with TO-1 biotin and fluorescence was analyzed at 6 h and days 1-12. As shown in FIG. 22, increased fluorescence/stability was detected from the circular aptamer, with fluorescence detected at least for 10 days in culture.

### Example 40: Circular RNA Bound Protein and RNA

This Example demonstrates circular RNA binding to protein and RNA for sequestration.

Human antigen receptor (HuR) can be a pathogenic protein, e.g., it is known to bind and stabilize cancer related mRNA transcripts, such as mRNAs for proto-oncogenes, cytokines, growth factors, and invasion factors. HuR has a central tumorigenic activity by enabling multiple cancer phenotypes. Sequestration of HuR with circular RNA may attenuate tumorigenic growth in multiple cancers.

RNA plays a central role in cell metabolism and RNA molecules undergo multiple post-transcriptional processes, such as splicing, editing, modification, translation, and degradation.

As shown in the following Example, circular RNA binds to HuR and RNA for sequestration.

Circular RNA was designed to include the HuR RNA binding motif: 5′-UCAUAAUCAA UUUAUUAUUUUCUUUUAUUUUAUUCACAUAAUUUUGUUUUU-3′ (SEQ ID NO: 31) to competitively bind HuR and inhibit its binding/downstream functions and the RNA binding motif: 5′-CGA GAC GCT ACG GAC TTA AAA TCC GTT GAC-3′(SEQ ID NO: 32).

Unmodified linear RNA was synthesized by in vitro transcription using T7 RNA polymerase from a DNA segment comprising the HuR RNA motif and protein binding sequence.

Circular RNA was designed to include the HuR RNA binding aptamer motif: 5′-UCAUAAUCAA UUUAUUAUUUUCUUUUAUUUUAUUCACAUAAUUUUGUUUUU-3′ (SEQ ID NO: 31) to competitively bind HuR and inhibit its binding/downstream functions and the RNA binding aptamer motif: 5′-CGA GAC GCT ACG GAC TTA AAA TCC GTT GAC-3′ (SEQ ID NO: 32).

Unmodified linear RNA was synthesized by in vitro transcription using T7 RNA polymerase from a DNA segment comprising the HuR RNA motif and protein binding sequence.

Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

Circular RNA binding to HuR and RNA was evaluated in vitro by a combination of HuR immunoprecipitation (IP) and Biotin RNA pull-down assay, followed by qPCR. HuR protein-coupled to Protein G-anti HuR antibody was incubated with circular RNA, washed and eluted at low pH. Bound material was incubated with biotinylated RNA, washed and pulled down with streptavidin dynabeads.

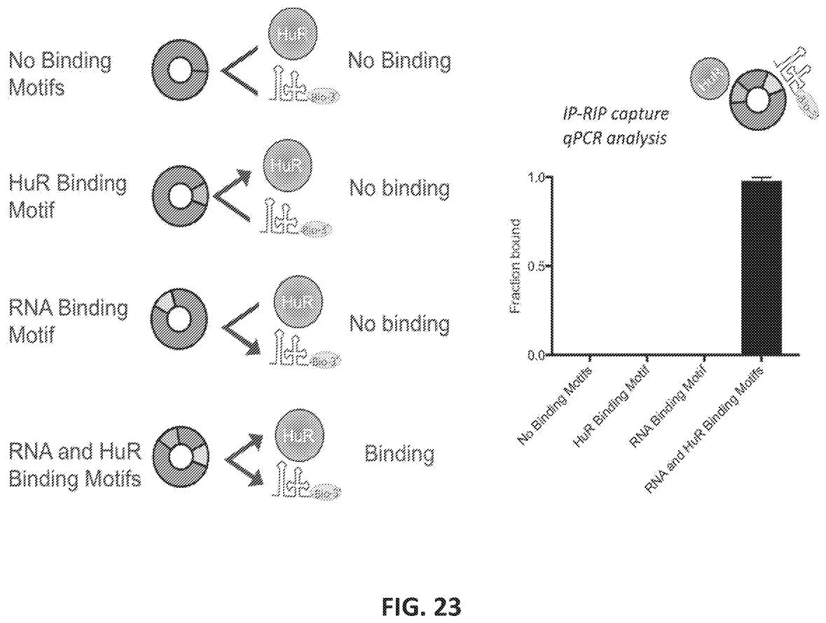

HuR bound circular RNAs with the HuR RNA binding aptamer motif and the streptavidin pull-down yielded RNAs with the RNA binding aptamer motifs as shown in FIG. 23. Thus binding was observed when the two, HuR and RNA, binding motifs were present. This result demonstrated that biomolecules of interests were selectively bound.

### Example 41: Circular RNA Bound Protein and DNA

This Example demonstrates circular RNA binding to protein and DNA for sequestration.

DNA binding by proteins and RNAs plays a pivotal role in different cellular processes, i.e., transcription.

Human antigen receptor (HuR) plays a central role in mRNA fate and plays a key role in post-transcriptional regulation of mRNA targets with central cellular functions, making it an important protein in pathogenesis. It is known to bind and stabilize cancer related mRNA transcripts, thus, HuR has a central tumorigenic activity by enabling multiple cancer phenotypes.

Targeting and competing these contacts with circular RNA could be used to modulate these interactions and control outcomes in disease and non-disease processes.

Circular RNA was designed to include the DNA binding aptamer motif: 5′-CGA GAC GCT ACG GAC TTA AAA TCC GTT GAC-3′ (SEQ ID NO: 32) RNA.

Unmodified linear RNA was synthesized by in vitro transcription using T7 RNA polymerase from a DNA segment. Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE.

Circular RNA binding to DNA and HuR was evaluated in vitro by a combination of HuR immunoprecipitation (IP) and biotinylated DNA pull-down assay, followed by RT-qPCR. Circular RNA lacking the DNA binding motif or HuR motif was used as a specificity control. The biotinylated DNA bound circular RNAs with the DNA binding aptamer motif.

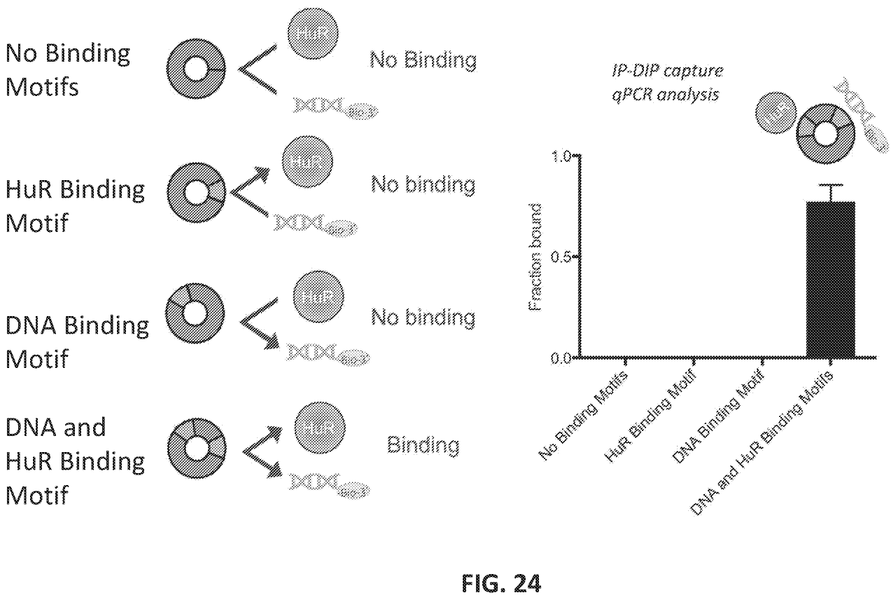

HuR protein-coupled to Protein G-anti-HuR beads was incubated with the circular RNA, washed and eluted at low pH. Bound material was incubated with biotinylated DNA, washed and pulled down with streptavidin Dynabeads. HuR bound circular RNAs with the HuR DNA binding aptamer motif and the streptavidin pull-down yielded RNAs with the DNA binding aptamer motifs as shown in FIG. 24. Thus, binding was observed when the two, HuR and DNA, binding aptamer motifs were present. This result demonstrated protein and DNA molecules of interests were selectively bound to the same circular construct.

### Example 42: Circular RNA Translated a Protein, and Bound to a Different Protein that Affected its Translation

This Example demonstrates circular RNA encoding a protein and binding a different protein that has an effect in circular RNA translation.

Human antigen receptor (HuR) plays a central role in mRNA fate and plays a key role in post-transcriptional regulation of mRNA targets with central cellular functions. Thus, using HuR to control RNA expression may provide control over translated protein dosage.

As shown in the following Example, a non-naturally occurring circular RNA was engineered to encode Gaussia Luciferase (GLuc), a biologically active secreted protein and to bind HuR to regulate GLuc translation. This circular RNA included an IRES, an ORF encoding Gaussia Luciferase, two spacer elements flanking the IRES-ORF and 1×, 2× or 3×HuR binding aptamer motifs: 5′-UCA UAA UCA AUU UAU UAU UUU CUU UUA UUU UAU UCA CAU AAU UUU GUU UUU-3′ (SEQ ID NO: 33), 5′-AUU UUG UUU UUA ACA UUUC-3′ (SEQ ID NO: 34), 5′-UCA UAA UCA AUU UAU UAU UUU CUU UUA UUU UAU UCA CAU AAU UUU GUU UUU AUU UUG UUU UUA ACA UUU C-3′ (SEQ ID NO: 35) to bind HuR.

Unmodified linear RNA was synthesized by in vitro transcription using T7 RNA polymerase from a DNA segment comprising the HuR RNA motif and protein binding sequence.

Transcribed RNA was purified with an RNA cleanup kit (New England Biolabs, T2050), treated with RNA 5′-phosphohydrolase (RppH, New England Biolabs, M0356) following the manufacturer's instructions, and purified again with the RNA purification column. RppH treated RNA was circularized using a splint DNA complementary to the circularization sequences and T4 RNA ligase 2 (New England Biolabs, M0239). Circular RNA was Urea-PAGE purified, eluted in a buffer containing (0.5M Sodium Acetate, 0.1% SDS, 1 mM EDTA, ethanol precipitated and resuspended in RNase free water. RNA quality was assessed by Urea-PAGE or through automated electrophoresis (Agilent).

Circular RNA binding to HuR was determined by in vitro RNA pull-down assay as described previously.

To evaluate the effect of HuR binding and its effect on circular RNA protein expression in cells, 5×10³ HeLa cells were successfully reverse transfected with a lipid-based transfection reagent (Invitrogen) and 2 nM of circular RNA. Gaussia Luciferase activity was monitored daily for up to 96 h in cell culture supernatants, as a measure of expression, using a Gaussia Luciferase assay kit and following manufacturer's instructions.

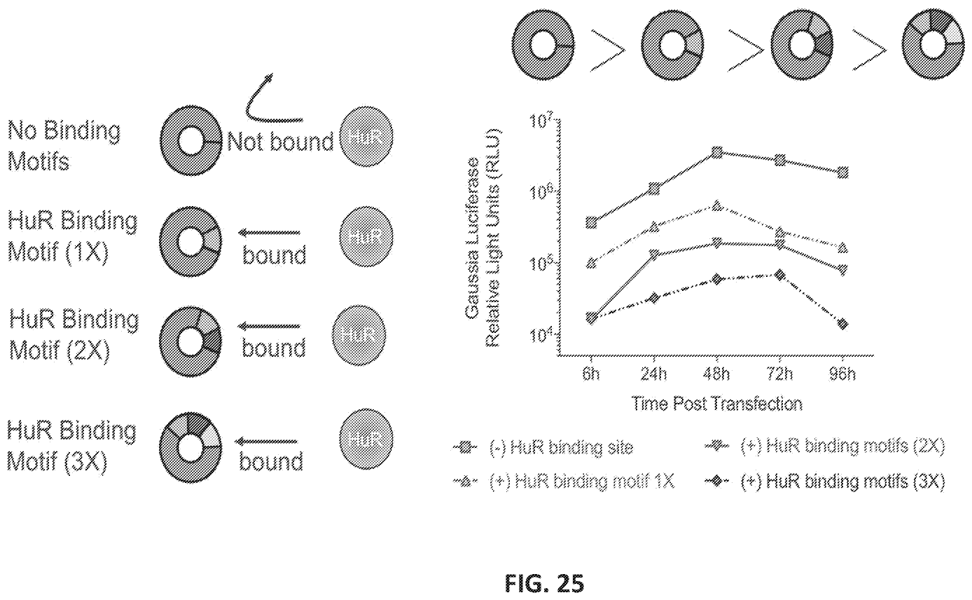

FIG. 25 shows lower secreted protein expression from circular RNA with HuR binding aptamer sites. Even more, the GLuc expression levels changed with the number of HuR binding aptamer motifs in the circular RNA. This example demonstrates that the level of translation from the engineered circular RNA was affected by additional protein binding aptamers.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed in practicing the disclosure. It is intended that the following claims define the scope of the disclosure and that methods and structures within the scope of these claims and their equivalents be covered thereby.

What is claimed is:

1. A method of binding a target in a cell, the method comprising:
providing a translation incompetent circular polyribonucleotide comprising an aptamer sequence, wherein the aptamer sequence has a secondary structure that binds the target; and
delivering the translation incompetent circular polyribonucleotide to the cell, wherein the translation incompetent circular polyribonucleotide forms a complex with the target detectable at least 5 days after delivery.

2. The method of claim 1, wherein the target is selected from the group consisting of a nucleic acid molecule, a small molecule, a protein, a carbohydrate, and a lipid.

3. The method of claim 1, wherein the target is a gene regulation protein.

4. The method of claim 3, wherein the gene regulation protein is a transcription factor.

5. The method of claim 2, wherein the nucleic acid molecule is a DNA molecule or an RNA molecule.

6. The method of claim 1, wherein the complex modulates gene expression.

7. The method of claim 1, wherein the complex modulates directed transcription of a DNA molecule, epigenetic remodeling of a DNA molecule, or degradation of a DNA molecule.

8. The method of claim 1, wherein the complex modulates degradation of the target, translocation of the target, or target signal transduction.

9. The method of claim 6, wherein the gene expression is associated with pathogenesis of a disease or condition.

10. The method of claim 1, wherein the complex is detectable at least 7, 8, 9, or 10 days after delivery.

11. The method of claim 1, wherein the translation incompetent circular polyribonucleotide is present at least five days after delivery.

12. The method of claim 1, wherein the translation incompetent circular polyribonucleotide is present at least 6, 7, 8, 9, or 10 days after delivery.

13. The method of claim 1, wherein the translation incompetent circular polyribonucleotide is an unmodified translation incompetent circular polyribonucleotide.

14. The method of claim 1, wherein the translation incompetent circular polyribonucleotide has a quasi-double-stranded secondary structure.

15. The method of claim 1, wherein the aptamer sequence further has a tertiary structure that binds the target.

16. The method of claim 1, wherein the cell is a eukaryotic cell.

17. The method claim 16, wherein the eukaryotic cell is a human cell.

18. A method of binding a transcription factor in a cell, the method comprising:
providing a translation incompetent circular polyribonucleotide comprising an aptamer sequence that binds the transcription factor; and
delivering the translation incompetent circular polyribonucleotide to the cell, wherein the translation incompetent circular polyribonucleotide forms a complex with the transcription factor and modulates gene expression.

19. A method of sequestering a transcription factor in a cell, the method comprising:
providing a translation incompetent circular polyribonucleotide comprising an aptamer sequence that binds the transcription factor; and
delivering the translation incompetent circular polyribonucleotide to the cell, wherein the translation incompetent circular polyribonucleotide sequesters the transcription factor by binding the transcription factor to form a complex in the cell.

20. The method of claim 19, wherein cell viability decreases after formation of the complex.

21. A method of sensitizing a cell to a cytotoxic agent, the method comprising:
providing a translation incompetent circular polyribonucleotide comprising an aptamer sequence that binds a transcription factor; and
delivering the cytotoxic agent and the translation incompetent circular polyribonucleotide to the cell, wherein the translation incompetent circular polyribonucleotide forms a complex with the transcription factor in the cell;
thereby sensitizing the cell to the cytotoxic agent compared to a cell lacking the translation incompetent circular polyribonucleotide.

22. The method of claim 21, wherein the sensitizing the cell to the cytotoxic agent results in decreased cell viability after the delivering of the cytotoxic agent and the translation incompetent circular polyribonucleotide.

23. The method of claim 22, wherein the decreased cell viability is decreased by 40% or more at least two days after the delivering of the cytotoxic agent and the translation incompetent circular polyribonucleotide.

24. A method of binding a pathogenic protein in a cell, the method comprising:
providing a translation incompetent circular polyribonucleotide comprising an aptamer sequence that binds the pathogenic protein; and
delivering the translation incompetent circular polyribonucleotide to the cell, wherein the translation incompetent circular polyribonucleotide forms a complex with the pathogenic protein for degrading the pathogenic protein.

25. A method of binding a ribonucleic acid molecule in a cell, the method comprising:
providing a translation incompetent circular polyribonucleotide comprising a sequence complementary to a sequence of the ribonucleic acid molecule; and
delivering the translation incompetent circular polyribonucleotide to the cell, wherein the translation incompetent circular polyribonucleotide forms a complex with the ribonucleic acid molecule.

26. A method of binding genomic deoxyribonucleic acid molecule in a cell, the method comprising:
providing a translation incompetent circular polyribonucleotide comprising an aptamer sequence that binds the genomic deoxyribonucleic acid molecule; and
delivering the translation incompetent circular polyribonucleotide to the cell, wherein the translation incompetent circular polyribonucleotide forms a complex with the genomic deoxyribonucleic acid molecule and modulates gene expression.

27. A method of binding a small molecule in a cell, the method comprising:
providing a translation incompetent circular polyribonucleotide comprising an aptamer sequence that binds the small molecule; and
delivering the translation incompetent circular polyribonucleotide to the cell, wherein the translation incompetent circular polyribonucleotide forms a complex with the small molecule and modulates a cellular process.

28. The method of claim 27, wherein the small molecule is an organic compound having a molecular weight of no more than 900 daltons and modulates a cellular process.

29. The method of claim 27, wherein the small molecule is a drug.

30. The method of claim 27, wherein the small molecule is a fluorophore.

31. The method of claim 27, wherein the small molecule is a metabolite.

32. A composition comprising a translation incompetent circular polyribonucleotide comprising an aptamer sequence, wherein the aptamer sequence has a secondary structure that binds a target.

33. A pharmaceutical composition comprising a translation incompetent circular polyribonucleotide comprising an aptamer sequence, wherein the aptamer sequence has a secondary structure that binds the target; and a pharmaceutically acceptable carrier or excipient.

34. A cell comprising the translation incompetent circular polyribonucleotide of claim 32.

35. A method of treating a subject in need thereof, comprising administering the composition of claim 32 or the pharmaceutical composition of claim 33.

36. A polynucleotide encoding the translation incompetent circular polyribonucleotide of claim 32.

37. A method of producing the translation incompetent circular polyribonucleotide of claim 32.
